(19) United States
(12) Patent Application Publication
(10) Pub. No.: US 2025/0099028 A1
(43) Pub. Date: Mar. 27, 2025

(54) AI-BASED CONTACTLESS SLEEP ANALYSIS METHOD AND REAL-TIME SLEEP ENVIRONMENT ADJUSTMENT METHOD

(71) Applicant: ASLEEP CO., LTD, Seoul (KR)

(72) Inventors: Dongheon LEE, Seongnam-si, Gyeonggi-do (KR); AHN Chee Eun, Seoul (KR); Su Young KWON, Seoul (KR); Yoonpyo KOO, Seoul (KR); Sang Hyun LEE, Seoul (KR); Jinhwan JUNG, Daejeon (KR); Chae Eun LEE, Seoul (KR); Junki HONG, Seoul (KR); Hye Ah PARK, Seoul (KR); Hyunggug KIM, Seoul (KR); So Ra KANG, Seoul (KR); Jaehyun BAE, Seoul (KR); Woojung LIM, Seoul (KR)

(21) Appl. No.: 18/832,844

(22) PCT Filed: Jan. 25, 2023
(86) PCT No.: PCT/KR2023/001135

§ 371 Date: Jul. 24, 2024

(30) Foreign Application Priority Data
Jan. 25, 2022 (KR) 10-2022-0010449
Jul. 21, 2022 (KR) 10-2022-0090144
Oct. 13, 2022 (KR) 10-2022-0131829
Oct. 31, 2022 (KR) 10-2022-0143037
Nov. 1, 2022 (KR) 10-2022-0143598
Nov. 21, 2022 (KR) 10-2022-0156817
Dec. 6, 2022 (KR) 10-2022-0168612
Dec. 27, 2022 (KR) 10-2022-0186414

(51) Int. Cl. A61B 5/00; A61B 5/08; A61M 21/02

(52) U.S. Cl. CPC A61B 5/4812; A61B 5/08; A61M 21/02

(57) ABSTRACT

An AI-based contactless sleep analysis system and method are disclosed. The sleep analysis system comprises: a smartphone that downloads a sleep analysis application from a server, collects and transmits sleep sound information of a user in real time to the server, and receives a report of AI-trained sleep analysis results from the server; and at least one smart home appliance that is spaced apart and located around the user, simultaneously collects and transmits the sleep sound information to the smartphone, and provides a customized sleep environment based on the analysis results to the user who is response to a control of the smartphone.

### TECHNICAL FIELD

The present disclosure relates to an AI-based contactless sleep analysis method of performing a sleep analysis, and a method of adjusting a real-time sleep environment.

### BACKGROUND ART

For true healthcare, it is necessary to monitor and manage 24 hours a day. This is because health monitoring and management is not a simple one-to-one match, but all elements thereof are complexly linked to one another.

Furthermore, maintaining and improving health can be achieved through various means such as exercise, diet, etc. However, sleep occupies about 30% or more of time during a day. Therefore, managing sleep well is paramount in how to maintain and improve health.

However, machines have replaced simple labor of modern people, and modern people are enjoying a leisurely life. Nevertheless, modern people cannot get a good night's sleep due to irregular eating habits, living habits, and stress. Modern people suffer from sleep disorders such as insomnia, hypersomnia, sleep apnea syndrome, nightmares, night terrors, sleepwalking, etc.

Due to such increasingly serious sleep problems, needs for sleep health management increase. Accordingly, the sleep tech market, which aims to solve sleep problems with technology, is also growing rapidly.

According to the National Health Insurance Service, the number of patients with sleep disorders in the Republic of Korea increased by about 8% annually from 2014 to 2018, and about 570,000 patients were treated for sleep disorders in the Republic of Korea in 2018.

Korean Patent Application Publication No. 2003-0032529 discloses a sleep induction device and a sleep induction method enabling optimal sleep induction. The sleep induction device and the sleep induction method receive a user's body information and repeatedly learns according to the user's body state during sleep. In addition, it outputs vibration and/or ultrasound of a frequency band detected by repetitive learning to enable optimal sleep induction.

However, the prior art has concerns that the quality of sleep may be deteriorated due to discomfort caused by body-worn equipment, and periodic management of the equipment (e.g., charging, etc.) is required.

In addition, a sleep analysis method using a conventional wearable device has a problem in that sleep analysis is impossible when the wearable device does not properly contact the user's body. Furthermore, there is a problem in that sleep analysis is impossible when the user does not wear the wearable device.

In addition, when a plurality of users sleeps in the same room, sleep analysis of a wearable device wearer may be hindered due to movements of a wearable device non-wearer. Also, there is a problem that it is impossible to analyze sleep of the wearable device non-wearer.

In addition, the sleep analysis method or non-contact sleep management studies using the conventional wearable device use variance values of heart rate variability (HRV) or change values of brain waves when sleeping and being awake. However, the difference was not large, so there was a limit to measure wake time accurately, which is a basis of all sleep treatment.

Especially, when using changes in brain waves to treat sleep disorders such as snoring, precursor symptoms of snoring are not detected by changes in brain waves at all. Therefore, it is impossible to use changes in brain waves to prevent snoring. Furthermore, there is a limit of being used only in diagnosis of snoring, since subsequent changes of brain waves are detected after a patient's snoring.

Accordingly, a user's sleep state is estimated by monitoring degrees of activation of his/her autonomic nervous system according to breathing patterns and body movements during a night in a non-contact manner in recent years. Furthermore, studies are being conducted to adjust a user's sleep environment according to the estimated sleep state.

Especially, according to a number of papers that have studied relations between a sleep environment such as air quality, temperature, humidity, etc. and sleep, it has been confirmed that the sleep environment such as air quality, temperature, humidity, etc. has a decisive effect on sleep quality. This means that the sleep environment needs to be optimized in order to improve the sleep quality.

### DETAILED DESCRIPTION OF THE INVENTION

### Technical Tasks

An object of the present disclosure is to provide a sleep analysis system and method without purchasing or wearing a wearable device separately so that sleeps of various types of users can be analyzed accurately and conveniently in real time, regardless of time and place.

In addition, an object of the present disclosure is to replace various conventional bio-signals only with a user's breathing sound using a smart home appliance and a smartphone with a built-in microphone simultaneously, and provide a sleep analysis system and method capable of in-depth analysis of the user's sleep through artificial intelligence learning.

In addition, the present disclosure is to provide a variety of home appliances for providing an optimal sleep environment of a user which is related to various factors such as air quality, temperature or/and humidity based on the sleep state information sensed in the sleep environment.

The tasks to be resolved by the present disclosure are not limited to the aforementioned, and others not mentioned will be clearly understood by a person skilled in the art from the description below.

### Technical Solutions

Other specific details of the invention are included in the detailed description and drawings.

An AI-based non-contact sleep analysis system according to the present invention for achieving the object includes: a smartphone that downloads a sleep analysis application from a server, collects a user's sleep sound information in real time, transmits it to the server, and receives a sleep analysis result report learned by artificial intelligence from the server; and at least one smart home appliance that is located at a distance around the user and simultaneously collects the sleep sound information and transmits it to the smartphone, and provides a customized sleep environment to the user in response to a control of the smartphone.

A smart home appliance of an AI-based non-contact sleep analysis system according to the present invention for achieving the object includes: a sensor unit that collects a user's sleep sound information through a built-in microphone module: a memory for storing a program for performing a sleep analysis: a processor that reads a program stored in the memory, extracts a sleep analysis model, and analyzes the user's sleep based on the sleep sound information using the sleep analysis model; and an alarm unit transmitting tactile or auditory stimuli to the user when a sleep disorder occurs during the sleep analysis.

The sleep analysis result report of the AI-based non-contact sleep analysis system according to the present invention for achieving the object includes bed time, sleep latency time, sleep time, and time taken to wake up after an alarm.

The processor of the AI-based non-contact sleep analysis system according to the present invention for achieving the object monitors the user's sleep apnea in real time based on the sleep analysis.

The AI-based non-contact sleep analysis system according to the present invention for achieving the object further includes a communication unit for performing data transmission and reception with the smartphone and the server through a wireless communication network.

The processor of the AI-based non-contact sleep analysis system according to the present invention for achieving the object converts raw data of the user's sleep sound information into a spectrogram, and performs a second sleep analysis by inputting the spectrogram into the sleep analysis model modeled through deep learning.

An AI-based non-contact sleep analysis method according to the present invention for achieving the object includes steps of: by a smartphone, downloading a sleep analysis application from a server: by at least one smart home appliance, collecting a user's sleep sound information in real time and transmitting the user's sleep sound information to the server: by the smartphone, collecting the user's sleep sound information in real time and transmitting the user's sleep sound information to the server: by the smartphone, outputting a control signal controlling an operation of the at least one smart home appliance; and by the at least one smart home appliance, providing a customized sleep environment to the user.

The server of the AI-based non-contact sleep analysis method according to the present invention for achieving the object is an artificial intelligence server.

An AI-based non-contact sleep analysis method according to the present invention for achieving the object includes steps of: (a) determining whether a microphone is built into at least one smart home appliance (S7000): (b) by a smartphone, downloading a sleep analysis application from a server if a determination result of step (a) is positive (S7100): (c) determining whether the smart home appliance can adjust a sleep environment if the sleep analysis application is downloaded (S8000): (d) determining whether the smart home appliance is a device capable of providing data based on a sleep analysis if a determination result of step (c) is negative (S9000); and (e) operating the sleep analysis application if a determination result of step (d) is positive (S9100).

The step (b) of the AI-based non-contact sleep analysis method according to the present invention for achieving the object further includes a step of linking an application pre-installed in the smartphone with the sleep analysis application if the determination result of step (a) is negative (S7200).

The step (d) of the AI-based non-contact sleep analysis method according to the present invention for achieving the object further includes a step of creating a research interaction at the same time a sleep track application is activated if the determination result of step (c) is positive (S8100).

The step (d) of the AI-based non-contact sleep analysis method according to the present invention for achieving the object further includes a step of determining whether the smart home appliance is a device capable of providing data based on the sleep analysis through a user interface if the determination result of step (c) is negative (S9000).

In the step (c) of the AI-based non-contact sleep analysis method according to the present invention for achieving the object, the sleep environment includes any one or more of temperature, humidity, light, sounds, head and body positions, and scent.

The smart home appliance reaching the step (S8100) of the AI-based non-contact sleep analysis method according to the present invention for achieving the object includes at least one of an air conditioner, an air purifier, a humidifier, a dehumidifier, a blind, a curtain, a light, a smart speaker, a smart bed, a smart diffuser, and a smart device on which a healthcare application is installed.

The smart home appliance reaching the step (S9100) of the AI-based non-contact sleep analysis method according to the present invention for achieving the object includes at least one of a TV set, a clothes manager, a robot vacuum cleaner, a washing machine, a dryer, a refrigerator, and a smart device on which a healthcare application is installed.

The air purifier according to the present invention for achieving the object includes a network unit that receives environment sensing information from a user terminal, a processor that obtains sleep state information based on the environment sensing information and generates environment adjustment information using the sleep state information, and an operating unit that controls air quality in a sleep space based on the environment adjustment information.

In addition, the air purifier may further include a measurement unit that measures air components in the sleep space, and the processor may generate the measured air components and the environment adjustment information based on the environment sensing information.

An air conditioner according to the present invention for achieving the object includes a network unit that receives environment sensing information from a user terminal, a processor that obtains sleep state information based on the environment sensing information and generates environment adjustment information using the sleep state information, and an operating unit that controls temperature or/and humidity in a sleep space based on the environment adjustment information.

In addition, the air conditioner may further include a measurement unit that measures temperature or/and humidity in the sleep space, and the processor may generate the environment adjustment information based on the measured temperature or/and humidity and the environment sensing information.

### Technical Effects

According to one embodiment according to the present invention, a user's wake time and/or sleep state information can be predicted. Therefore, it is possible to analyze sleeps of various users conveniently and accurately at home regardless of time and place.

In addition, it is not necessary to wear a wearable device when analyzing a user's sleep, so the user's body freedom can be increased during sleep time.

In addition, it is possible to build sleep sound data by collecting polysomnography results from all over the world, and to make a sound AI serve as a new standard for home environment sleep tracking that verifies various races, ages, genders, and measurement environment.

In addition, it is possible to build an AI sleep stage analysis model by learning various ambient noises including noises that occur routinely in the surrounding space of a user's sleep environment, noises that occur abnormally or intermittently, etc.

In addition, it is possible to build sound AI and wireless communication sensing clinical data sets by utilizing smartphone sound data and smart speaker sound data collected simultaneously with polysomnography of multiple clinical subjects collected over a long period of time.

In addition, it is possible to analyze a user's sleep in depth using a smart appliance and a smartphone, and to perform not only a single person sleep analysis but also a multi-person sleep analysis.

In addition, it is possible to alleviate a sleep disorder appropriately when a user's sleep disorder occurs. Furthermore, when a plurality of people sleeps in the same place, it is possible to prevent other people's sleep disturbance by delivering an alarm for relieving a sleep disorder only to a user who has the sleep disorder.

In addition, it is possible to monitor a user's physical activity states in real time for 24 hours using a smart home appliance and/or a smartphone.

In addition, according to an embodiment of the present invention, it is possible to provide an optimized sleep environment for improving a user's sleep quality through sleep state information sensed in relation to the user's sleep environment.

Especially, it is possible to improve the sleep quality significantly by providing an optimal sleep environment related to various factors such as air quality, temperature or/and humidity.

The effects of the present disclosure are not limited to the effects mentioned above, and other effects not mentioned will be clearly understood by those skilled in the art from the description below.

### BRIEF DESCRIPTION OF THE DRAWINGS

FIG. 1 (a) is a conceptual diagram illustrating a system capable of implementing various aspects of a computing device for adjusting a sleep environment based on sleep state information related to one embodiment of the present invention.

FIG. 1 (b) is a conceptual diagram illustrating a system capable of implementing various aspects of a sleep environment adjustment device related to another embodiment of the present invention.

FIG. 1 (c) is a conceptual diagram illustrating a system capable of implementing various aspects of various electronic devices related to another embodiment of the present invention.

FIG. 2 is a block diagram of a computing device for adjusting a sleep environment based on sleep state information related to one embodiment of the present invention.

FIG. 3 is a diagram comparing a polysomnography (PSG) result and an analysis result using AI algorithms according to the present invention.

FIG. 4 is a diagram comparing a polysomnography (PSG) result and an analysis result using AI algorithms according to the present invention related to sleep apnea and hypopnea.

FIG. 5 is an exemplary diagram to illustrate a process for acquiring sleep sound information from environment sensing information related to one embodiment of the present invention.

FIG. 6 (a) is an exemplary diagram to illustrate a method for acquiring a spectrogram corresponding to sleep sound information related to one embodiment of the present invention.

FIG. 6 (b) is a conceptual diagram to illustrate a privacy protection method using a Mel spectrogram conversion for sleep sound information extracted from a user in a sleep analysis method according to the present invention.

FIG. 7 is an exemplary diagram illustrating time-specific environment adjustment information according to a sleep state of a user related to one embodiment of the present invention.

FIG. 8 is an exemplary flowchart for providing a method for providing a sleep adjustment environment based on sleep state information related to one embodiment of the present invention.

FIG. 9 is a schematic diagram illustrating one or more network functions related to one embodiment of the present invention.

FIG. 10 is an example block diagram illustrating a sleep environment adjustment device related to one embodiment of the present invention.

FIG. 11 (a) is an exemplary block diagram of a receiving module and a transmitting module related to one embodiment of the present invention.

FIG. 11 (b) is a block diagram illustrating a configuration of a smart home appliance in an AI-based contactless sleep analysis system according to the present invention.

FIG. 12 is an exemplary diagram illustrating a second sensor unit for detecting whether a user is located in a preset area related to one embodiment of the present invention.

FIG. 13 is an exemplary flowchart illustrating a process for generating sleep state information through an automatic sleep measurement mode related to one embodiment of the present invention.

FIG. 14 is a flowchart illustrating an exemplary process for adjusting an environment to induce a user to enter sleep related to one embodiment of the present invention.

FIG. 15 is a flowchart exemplarily illustrating a process for altering a user's sleep environment during sleep and immediately before waking up related to one embodiment of the present invention.

FIGS. 16 (a) and (b) are conceptual diagrams illustrating an operation of an air conditioner according to one embodiment of the present invention.

FIGS. 16 (c) and (d) are conceptual diagrams illustrating an operation of an air purifier according to one embodiment of the present invention.

FIG. 17 (a) is a block diagram illustrating a configuration of an air conditioner according to one embodiment of the present invention.

FIG. 17 (b) is a block diagram illustrating a configuration of an air purifier according to one embodiment of the present invention.

FIG. 18 is a diagram illustrating one example of the air conditioner illustrated in FIGS. 16 and 17.

FIG. 19 is a diagram illustrating another example of the air conditioner illustrated in FIGS. 16 and 17.

FIGS. 20 and 21 are diagrams illustrating another example of the air conditioner illustrated in FIGS. 16 and 17.

FIG. 22 is a drawing illustrating another example of the air conditioner illustrated in FIGS. 16 and 17.

FIGS. 23 (a) and (b) are diagrams illustrating a method of operating an indoor unit (500″) into a sleep mode via a display unit (570″) of the indoor unit (500″) illustrated in FIGS. 20 and 21.

FIGS. 23 (c) and (d) are drawings of a display unit (4000) to illustrate a sleep mode of an air purifier (700″) according to an embodiment of the present invention.

FIG. 24 (a) is a diagram illustrating a method of operating an indoor unit (500′, 500″, 500′″, 500″″) illustrated in FIGS. 18 to 22 in a sleep mode via a remote control (600) according to one embodiment of the present invention.

FIG. 24 (b) is a diagram illustrating an example of a display unit (4000) of an air purifier (700′) according to one embodiment of the present invention.

FIGS. 25 (a) and (b) are diagrams illustrating a method of driving the indoor unit (500′, 500″, 500′″, 500″″) illustrated in FIGS. 18 to 22 in a sleep mode via a user terminal (1) according to one embodiment of present invention.

FIG. 25 (c) is a diagram illustrating a screen of a first application to remotely control an air purifier (700″) from a user terminal (10) according to one embodiment of the present invention.

FIG. 25 (d) is a diagram illustrating a screen of an application that controls a sleep mode of an air purifier (700″) according to one embodiment of the present invention.

FIG. 26 is a diagram illustrating a method of operating an indoor unit or air purifier into a sleep mode automatically.

FIGS. 27 and 28 are diagrams illustrating a point in time of the sleep mode operation illustrated in FIG. 26.

FIGS. 29 (a) and (b) are diagrams illustrating an example of the air purifier illustrated in FIGS. 16 and 17.

FIG. 30 is a diagram illustrating a portion of the cover (1100, 2100) of the air purifier (700′) illustrated in FIG. 29 removed.

FIG. 31 (a) is a diagram illustrating another example of the air purifier illustrated in FIGS. 16 and 17.

FIG. 31 (b) is a diagram illustrating a point in time of a sleep mode operation of the air purifier (700″″) illustrated in FIG. 26.

FIG. 32 (a) is a diagram illustrating a sleep stage analysis using a spectrogram in a sleep analysis method according to the present invention.

FIG. 32 (b) is a diagram illustrating sleep disorder determination using a spectrogram in a sleep analysis method according to the present invention.

FIG. 33 (a) is a diagram illustrating an experimental process to verify performance of a sleep analysis method according to the present invention.

FIG. 33 (b) is a graph verifying performance of a sleep analysis method according to the present invention, comparing a polysomnography result (PSG result) and an analysis result (AI result) using an AI algorithm.

FIG. 34 is a table that verifies accuracy of a sleep analysis method according to the present invention, and an experimental result data analyzed by age, gender, BMI, and diseases status.

FIG. 35 is a conceptual diagram illustrating a sleep analysis method using a smart speaker and a smartphone according to one embodiment of the present invention.

FIG. 36 (a) is a flowchart illustrating a method for preventing and mitigating sleep disorders using an AI-based contactless sleep analysis system according to one embodiment of the present invention.

FIG. 36 (b) is a flowchart illustrating a method for preventing and alleviating sleep disorders using an AI-based contactless sleep analysis system according to another embodiment of the present invention.

FIG. 37 is a diagram illustrating a traffic response when a sleep analysis method according to the present invention is perform in a cloud.

FIG. 38 is a conceptual diagram illustrating single-person sleep analysis and multi-person sleep analysis in the sleep analysis method according to the present invention.

FIG. 39 is a flowchart illustrating an operation of an AI-based contactless sleep analysis method according to the present invention.

FIG. 40 is a flowchart illustrating embodiments of various smart appliances used in a sleep analysis method according to the present invention.

FIG. 41 is a table describing exemplary operations of a bedtime preparation stage among specific scenarios of a plurality of smart home appliances chronologically operating according to a user's sleep stages using a sleep analysis method according to the present invention.

FIG. 42 is a table describing exemplary operations after a falling asleep stage and before a deep sleep stage among the scenarios, which is chronologically connected to FIG. 41.

FIG. 43 is a table describing exemplary operations after the deep sleep stage and before a wake-up sensing stage among the scenarios, which is chronologically connected to FIG. 42.

FIG. 44 is a table describing exemplary operations of a wake-up stage among the scenarios, which is chronologically connected to FIG. 43.

FIG. 45 is a conceptual diagram illustrating a training method using only polysomnography microphone data(S) in a hospital environment according to a conventional sleep analysis method in order to compare a sleep analysis method of the present invention with the conventional sleep analysis method.

FIG. 46 is a conceptual diagram of a method for generating an AI sleep analysis model by reflecting various sounds in a home environment into the training method illustrated in FIG. 45 according to the sleep analysis method of the present invention.

FIG. 47 is a table that verifies performance of a sleep analysis method according to the present invention by dividing and training the performance into nine groups according to residential noise types.

FIG. 48 is a schematic diagram to illustrate a 24-hour monitoring process of a user by an AI-based contactless sleep analysis system and a sleep analysis method according to the present invention.

FIG. 49 is a table of mean-per-class results comparing smart home appliances and sleep analysis methods according to the present invention with existing products and devices from the world's leading sleep technology companies.

FIG. 50 is a block diagram illustrating operations of an AI-based contactless sleep analysis system according to one embodiment of the present invention.

FIG. 51 is a block diagram illustrating operations among components of an AI-based contactless sleep analysis system according to one embodiment of the present invention.

FIG. 52 is a table describing exemplary operations by a location where each environment adjustment device is placed, an activation status according to sleep state information for each specific device, a sleep mode, and a wake-up mode.

### DESCRIPTION OF EMBODIMENTS

The advantages and features of the present invention, and methods of achieving them will become clearer by referring to the following detailed description of embodiments and accompanying drawings.

However, the present invention is not limited to the embodiments disclosed below and may be implemented in a variety of different forms. These embodiments are provided only to make the disclosure of the present invention complete, and to fully inform those skilled in the art to which the present invention pertains of the scope of the present invention, and the present invention is defined solely by the scope of the claims.

In describing the embodiments, such detailed description is omitted where it is determined that a detailed description of the relevant prior art would obscure the essence of the embodiments disclosed herein. Furthermore, the attached drawings are intended only to facilitate understanding of the embodiments disclosed herein. The technical ideas disclosed herein are not limited by the attached drawings and should be understood to include all modifications, equivalents, and substitutions that are within the scope of the ideas and technology of the present invention.

Terms used herein is for describing the embodiments and is not intended to limit the present invention.

Unless otherwise defined, all terms (including technical and scientific terminology) used in this specification may be used with meanings commonly understood by those skilled in the art to which the present invention pertains. Furthermore, generally used dictionary-defined terms are not idealized or over-interpreted unless they are specifically defined.

In this specification, singular forms also include plural forms unless specifically stated otherwise in a phrase or sentence. As used herein, “comprises” and/or “comprising” does not exclude the presence or addition of one or more elements other than the recited elements. The same reference numerals throughout the specification refer to the same elements, and “and/or” includes each and every combination of one or more of the recited elements. Although “first”, “second”, etc. are used to describe various elements, these elements are not limited by these terms, of course. These terms are only used to distinguish one element from another. Accordingly, it goes without saying that the first element mentioned below may also be the second element within the technological concepts of the present invention.

The term “unit” or “module” used in specification means a software component and/or a hardware component such as FPGA or ASIC, and “unit” or “module” performs certain roles. However, “unit” or “module” is not meant to be limited to software or hardware. A “unit” or “module” may be configured to reside in an addressable storage medium and may be configured to run one or more processors. Thus, as an example, a “unit” or “module” may refer to components such as software components, object-oriented software components, class components, and task components, as well as processes, functions, properties, procedures, subroutines, segments of program code, drivers, firmware, microcode, circuitry, data, databases, data structures, tables, arrays and variables. Functions provided within components and “units” or “modules” may be combined with smaller numbers of components and “units” or “modules”, or may be divided into additional components and “units” or “modules”.

In this specification, a computer means any kind of hardware device including at least one processor, and may be understood as encompassing a software configuration operating in a corresponding hardware device according to an embodiment. For example, a computer may be understood as including a smartphone, a table PC, a desktop computer, a laptop computer, and user clients and applications run on each device, but it is not limited thereto.

In addition, a “smart home appliance” described below is a device with a built-in microphone capable of detecting a user's breathing sound and collecting sound data, and may include a smart speaker, a smart TV, a smart light, a smart mattress, etc.

In addition, a “sleep track application” may refer to an application that transmits a user's sleep report to a smartphone using a PUI, VUI and GUI, and operates a smart home appliance according to a report result.

In addition, a “research interaction” may refer to research and development of a new product for improving sleep quality of a user in a corresponding category such as fragrance, cosmetics, food, health, health supplements, and hormone.

In addition, a “research interaction of the sleep track application” may refer to developing sleep environment adjustment services and new products to improve sleep quality based on a sleep analysis analyzed in the sleep track application.

In addition, a “sleep management application interaction” may refer to interaction between a traditional sleep industry that enables sleep storytelling, a related industry such as sports, hotels, entering university academies, the military, and a sleep management application that enables a sleep analysis without a hardware solution.

In addition, an “interaction from research interaction to a sleep management application” may refer to interaction between a new product without a digital product and a sleep management application capable of a sleep analysis without a hardware solution.

Those skilled in the art should recognize that the various exemplary logical blocks, components, modules, circuits, means, logics, and algorithm steps described in connection with the embodiments disclosed herein may be implemented in electronic hardware, computer software, or combinations of both. To clearly illustrate the interchangeability of hardware and software, various illustrative components, blocks, configurations, means, logics, modules, circuits, and steps have been described above generally in terms of their functionality. Whether such functionality is implemented as hardware or as software depends on design constraints imposed on the particular application and the overall system. Skilled artisans may implement the described functionality in varying ways for each particular application. However, such implementation decisions should not be interpreted as causing a departure from the scope of the present invention.

Hereinafter, embodiments of the present invention will be described in detail with reference to the accompanying drawings.

The individual steps described in this specification is described as being performed by a computer, but the subject of each step is not limited thereto. Depending on embodiments, at least some of the steps may be performed in different devices.

### Overall Configurations

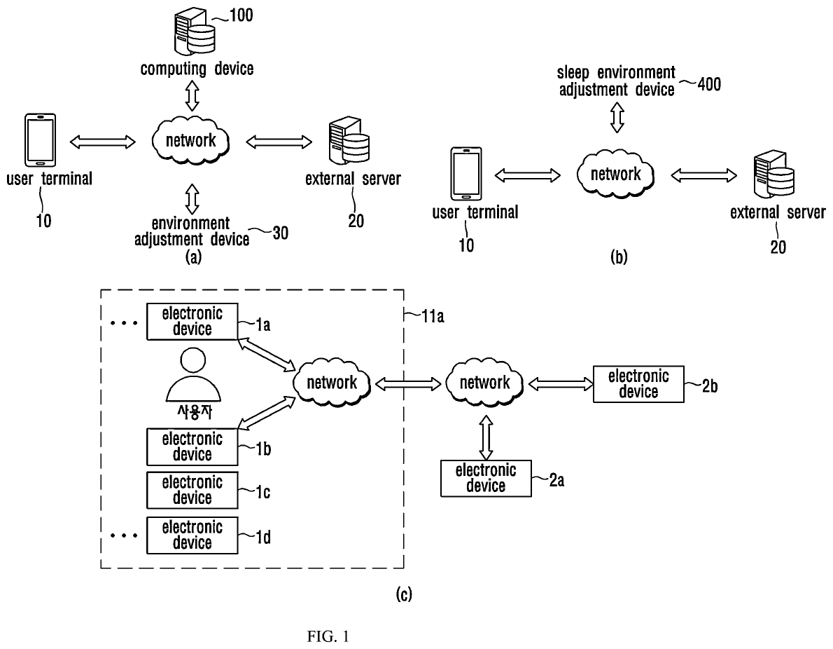

FIG. 1 (a) is a conceptual diagram illustrating a system in which various aspects of a computing device for adjusting a sleep environment may be implemented based on sleep state information related to one embodiment of the present disclosure. The system according to embodiments of the present disclosure may include a computing device (100), a user terminal (10), an external server (20), an environment adjustment device (30), and a network. Here, a system for implementing a method for adjusting a sleep environment based on sleep state information shown in FIG. 1 (a) is according to one embodiment. The components of the system are not limited to the embodiment shown in FIG. 1, and the components may be added, changed, or deleted as needed.

On the one hand, FIG. 1 (b) is a conceptual diagram of a system in which various aspects of a sleep environment adjustment device may be implemented related to another embodiment of the present disclosure.

A system according to embodiments of the present disclosure may include a sleep environment adjustment device (400), a user terminal (10), an external server (20), and a network. Here, a system for implementing a method for adjusting a sleep environment based on the sleep state information illustrated in FIG. 1 (b) is according to one embodiment, and its components are not limited to the embodiment shown in FIG. 1 (b), and may be added, changed, or deleted as needed.

First, a system according to the embodiment illustrated in FIG. 1 (a) will be described.

As shown in FIG. 1 (a), the computing device (100), the user terminal (10), the external server (20), and the environment adjustment device (30) of the present invention can transmit and receive data for the system according to one embodiment of the present disclosure to each other via a network.

Networks according to embodiments of the present disclosure may utilize a variety of wireline communication systems, such as the public switched telephone network (PSTN), xDSL (x Digital Subscriber Line), RADSL (Rate Adaptive DSL), MDSL (Multi Rate DSL), VDSL (Very High Speed DSL), UADSL (Universal Asymmetric DSL), HDSL (High Bit Rate DSL), and local area networks (LANs). Furthermore, the network presented herein may use various wireless communication systems like other systems such as Code Division Multi Access (CDMA), Time Division Multi Access (TDMA), Frequency Division Multi Access (FDMA), Orthogonal Frequency Division Multi Access (OFDMA), and Single Carrier-FDMA (SC-FDMA).

A network according to embodiments of the present disclosure can be configured regardless of its communication aspects such as wired and wireless. A network according to embodiments of the present disclosure may comprise various communication networks, such as a personal area network (PAN), a wired area network (WAN), etc. Furthermore, the network may be the World Wide Web (WWW) which is publicly known. The network may also utilize wireless transmission technologies for short-range communication, such as Infrared Data Association (IrDA) or Bluetooth. The technologies described herein may be used in other networks as well as the networks mentioned above.

According to one embodiment of the present disclosure, the user terminal (10) is a terminal that can receive information related to a user's sleep through information exchange with a computing device (100), and may mean a terminal owned by the user. For example, a user terminal (10) may be a terminal associated with a user who wishes to improve health through information related to their sleep habits. The user may acquire monitoring information regarding his or her own sleep through the user terminal (10). For example, monitoring information related to a sleep may include sleep state information related to a time a user went to sleep, a length of time a user slept, and a time a user woke up, or sleep stage information related to changes in sleep stages during a sleep. As a specific example, sleep stage information may refer to information about whether a user's sleep was light sleep, moderate sleep, deep sleep, or REM sleep at each point during a user's last eight hours of sleep. The foregoing specific description of sleep stage information is by way of example only, and the present invention is not limited thereto.

In another aspect, FIG. 1 (c) is a conceptual diagram illustrating a system in which various aspects of various electronic devices related to another embodiment of the present disclosure can be implemented.

The electronic devices illustrated in FIG. 1 (c) may perform at least one of operations performed by various devices according to an embodiment of the present disclosure.

For example, operations performed by various devices according to an embodiment of the present disclosure include acquiring environment sensing information, learning a sleep analysis model, inferring a sleep analysis model, acquiring sleep state information, controlling an electronic device, displaying sleep state information, and displaying environment adjustment information.

Or, for example, they may include receiving information related to the user's sleep, transmitting or receiving environment sensing information, determining environment sensing information, processing data, processing services, providing services, analyzing a sleep state, building a training data set based on information related to a user's sleep, storing information about the acquired data or a plurality of training data for training a neural network, generating environment adjustment information, determining environment adjustment information, operating an environment adjustment module based on environment adjustment information, transmitting or receiving various information, or transmitting or receiving data for systems according to embodiments of the present disclosure over a network.

The electronic devices illustrated in FIG. 1 (c) may individually perform operations performed by various devices according to embodiments of the present disclosure, but the electronic devices may simultaneously or sequentially perform one or more operations.

Referring to FIG. 1 (c), the electronic devices (1a to 1d) may be electronic devices within a range of a preset area (11a), which is an area where object state information such as information about a user's movement or breathing can be obtained.

In another aspect, referring to FIG. 1 (c), the electronic devices (1a and 1d) may be devices comprising a combination of two or more electronic devices.

In another aspect, referring to FIG. 1 (c), the electronic devices (1a and 1b) may be electronic devices connected to a network within the preset area (11a).

In another aspect, referring to FIG. 1 (c), the electronic devices (1c and 1d) may be electronic devices not connected to a network within the preset area (11a).

In another aspect, referring to FIG. 1 (c), the electronic devices (2a and 2b) may be electronic devices outside the range of the preset area (11a).

In another aspect, referring to FIG. 1 (c), there may be a network that interacts with electronic devices within the range of the preset area (11a), and there may be a network that interacts with electronic devices outside the range of the preset area (11a).

Here, the network interacting with the electronic devices may serve to transmit and receive information for controlling a smart home appliance within the range of the preset area (11a).

In addition, for example, the network interacting with the electronic devices within the range of the preset area (11a) may be a local area network or a local network. Here, for example, the network interacting with the electronic devices within the range of the preset area (11a) may be a wide area network or a global network.

The specific description of an operation of the network illustrated in FIG. 1 (c) is the same as that described in the diagrams of FIG. 1 (a) or FIG. 1 (b). Therefore, redundant description will be omitted.

In another aspect, referring to FIG. 1 (c), there may be one or more electronic devices connected via a network outside the range of the preset area (11a). In this case, the electronic devices may distribute data to each other or perform one or more operations.

In addition, if there is at least one electronic device connected via a network outside the range of the preset area (11a), each electronic device may perform operations independently.

Hereinafter, referring to FIG. 1 (c), various aspects according to embodiments of the present disclosure will be described, but the present invention is not limited thereto.

For example, according to an embodiment of the present disclosure, the steps of acquiring environment sensing information, performing preprocessing on the acquired environment sensing information, converting sound information included in the preprocessed environment sensing information into a spectrogram, generating sleep state information based on the converted spectrogram, and controlling the electronic device so that an environment is adjusted based on the generated sleep state information may be performed in an electronic device equipped with environment sensing and control functions.

In addition, according to an embodiment of the present disclosure, the steps of acquiring environment sensing information, performing preprocessing on the acquired environment sensing information, converting sound information included in the preprocessed environment sensing information into a spectrogram, and transmitting the converted spectrogram to an AI server (310), when the AI server (310) generates sleep state information through learning or inference based on the transmitted spectrogram, receiving, by the electronic device, the sleep state information generated by the AI server (310), and controlling the electronic device based on the received sleep state information so that an environment is adjusted may be performed in an electronic device equipped with environment sensing and control functions.

In addition, according to an embodiment of the present disclosure, in an electronic device for controlling a home appliance, the steps of acquiring environment sensing information in the electronic device, performing preprocessing on the acquired environment sensing information, converting sound information included in the preprocessed environment sensing information into a spectrogram, generating sleep state information based on the converted spectrogram, and controlling the home appliance based on the generated sleep state information so that the electronic device can cause the home appliance to adjust an environment may be performed.

In addition, according to an embodiment of the present disclosure, in an electronic device for controlling a home appliance, the steps of acquiring environment sensing information, performing preprocessing on the acquired environment sensing information, converting sound information included in the preprocessed environment sensing information into a spectrogram, when transmitting the converted spectrogram to the AI server (310) is performed, and the AI server (310) generates sleep state information through learning or inference based on the transmitted spectrogram, receiving, by the electronic device, sleep state information generated by the AI server (310), and controlling the home appliance so that the electronic device can cause the home appliance to adjust an environment based on the received sleep state information may be performed.

In addition, according to an embodiment of the present disclosure, there is an electronic device for controlling a home appliance for adjusting an environment. When another electronic device acquires environment sensing information, converts sound information included in the acquired environment sensing information into a spectrogram, and generates sleep state information based on the converted spectrogram, the electronic device receives the sleep state information from the another electronic device, and controls the home appliance for the home appliance to adjust an environment based on the received sleep state information. These steps may be performed. Here, the another electronic device is a device different from the electronic device controlling the home appliance, which may correspond to one or more electronic devices. In case that the another electronic device is in plural, acquiring environment sensing information, converting the environment sensing information into a spectrogram of sound information included in the environment sensing information, and generating sleep state information may be performed independently.

For example, according to an embodiment of the present disclosure, there is an electronic device for controlling a home appliance for adjusting an environment. Another electronic device acquires environment sensing information, converts sound information included in the acquired environment sensing information into a spectrogram, transmit the converted spectrogram to an AI server (310), and the AI server (310) generates sleep state information based on the transmitted spectrogram. Subsequently, the electronic device receives the sleep state information generated by the AI server (310), and controls the home appliance based on the received sleep state information so as for the home appliance to adjust an environment. The above-described step may be performed. Here, since the description on the another electronic device is the same as the previously described, a duplicate description will be omitted.

In various embodiments of the present disclosure described above, various operations such as acquiring environment sensing information, preprocessing the environment sensing information, converting the environment sensing information to spectrograms, generating sleep state information, and controlling an electronic device or appliance (e.g., a smart home appliance) may occur on multiple devices, not necessarily on the same electronic device. This is an example to illustrate that they may occur chronologically, simultaneously, or independently or separately. Therefore, the present invention is not limited to the various embodiments described above.

Hereinafter, various operations according to the present invention will be described with specific examples. However, as described above, the examples of electronic devices to be described hereinafter are for illustrative purposes only and are not intended to be limited to the electronic devices that perform certain operations.

### Acquisition of Environment Sensing Information

In an embodiment, the environment sensing information of the present invention may be acquired through an electronic device (e.g., a user terminal (10), etc.). The environment sensing information may refer to sensing information acquired from a space in which a user is located. The environment sensing information may be sensing information acquired related to the user's activity or sleep by a non-contact method.

For example, the environment sensing information may be sleep sound information acquired in a bedroom where the user sleeps. According to an embodiment, the environment sensing information acquired via the user terminal (10) may be the information on which the present invention is based for acquiring the user's sleep state information. As a specific example, sleep state information related to whether the user is before sleeping, during sleeping, or after sleeping may be acquired through environment sensing information acquired in relation to the user's activity.

For example, the environment sensing information may include breathing and movement information of the user. For this purpose, the user terminal (10) may be equipped with a radar sensor as a motion sensor. The user terminal (10) may generate a discrete waveform (respiration information) corresponding to the user's breathing by processing the user's movement and distance measured through the radar sensor.

For example, the environment sensing information may include measurement values acquired through sensors that measure temperature, humidity, and light levels in a bedroom. For this purpose, the user terminal (10) may be equipped with sensors that measure temperature, humidity, and light levels in the bedroom.

Such user terminal (10) may refer to any type of entity or entities in the system that has a mechanism for communicating with the computing device (100). For example, such user terminal (10) may include a personal computer, notebook, mobile terminal, smartphone, tablet PC, artificial intelligence (AI) speaker and artificial intelligence (AI) TV, and wearable device, etc., and may include any type of terminal that has access to a wired or wireless network. The user terminal (10) may also include may also include any server implemented by at least one of an agent, an application programming interface (API), and a plug-in. Furthermore, the user terminal (10) may include an application source and/or a client application.

According to one embodiment of the present disclosure, the external server (20) may be a server that stores information about a plurality of training data for training a neural network. For example, the plurality of training data may include medical examination information, sleep test information, etc. For example, the external server (20) may be at least one of a hospital server and an information server. The external server (20) may be a server that stores information regarding a plurality of polysomnography records, electronic health records, and electronic medical records. For example, the polysomnography records may include information about breathing and movement during sleep of a sleep test subject, and information about sleep diagnostic results corresponding to the information (e.g., sleep stages, etc.). The information stored on the external server (20) may be utilized as training data, validation data, and test data for training the neural network of the present invention.

The computing device (100) of the present invention may receive medical examination information or sleep examination information etc. from the external server (20), and build a learning data set based on the information. By performing training on one or more network functions through the training data set, the computing device (100) can generate a sleep analysis model for acquiring sleep state information corresponding to environment sensing information. A specific description on a configuration for building a training data set for training a neural network of the present invention and a learning method utilizing the training data set will be described later.

The external server (20) is a digital device, which may be a computing-capable device having a processor and memory, such as a laptop computer, notebook computer, desktop computer, web pad, or mobile phone. The external server (20) may be a web server that processes services. The foregoing types of servers are only examples and the present invention is not limited thereto.

According to one embodiment of the present disclosure, the environment adjustment device (30) can adjust a user's sleep environment. Specifically, the environment adjustment device (30) may include one or more environment adjustment modules. The environment adjustment device (30) can adjust the user's sleep environment by operating the environment adjustment module regarding at least one of air quality, illumination, temperature, wind direction, humidity, and sound of a space in which the user is located based on environment adjustment information received from the computing device (100).

In addition, in an embodiment such as FIG. 1 (c), for example, at least one of the electronic devices illustrated in FIG. 1 (c) may perform the operations described above.

The environment adjustment device (30) may be implemented as a television that provides images and videos and generates sound, an air purifier that can control air quality, a lighting device that can control light intensity (illumination), a heating/air conditioning unit that can control temperature, an air conditioner that can control temperature and humidity, an audio/speaker that can control sound, a styler that can manage clothing, blinds or curtains, a robot or vacuum cleaner, a washer or dryer, a water purifier, an oven or range, etc.

The environment adjustment information may be a signal generated by the computing device (100) based on determination of information about a user's sleep state. For example, the environment adjustment information may include information about lowering or increasing light levels, etc. If the environment adjustment device (30) is a lighting device, the environment adjustment information may include control information to gradually increase 3000K white light from 0 lux to 250 lux illumination starting 30 minutes before a predicted wake-up event.

In a specific example, if the environment adjustment device (30) is an air purifier or air conditioner, the environment adjustment information may include adjusting temperature and/or humidity based on a user's real-time sleep state, removing particulate matter (fine, ultrafine, and extremely ultrafine particles), removing harmful gases, operating allergy care, deodorizing/sterilizing, dehumidifying/humidifying, adjusting blowing intensity, adjusting air purifier or air conditioner operating noise, turning on LEDs, managing smog causing substances (SO2, NO2), removing household odors, and various other information. Furthermore, if the environment adjustment device (30) is an air conditioner, the environment adjustment information may include adjusting temperature and humidity of a sleep space, adjusting blowing intensity, adjusting operating noise, turning on the LEDs, etc. based on real-time sleep states of the user.

As a further example, the environment adjustment information may include control information for adjusting at least one of temperature, humidity, wind direction, or sound. The foregoing specific descriptions of environment adjustment information are examples only, and the present invention is not limited thereto.

The one or more environment adjustment modules included in the environment adjustment device (30) may include, for example, at least one of a light control module, a temperature control module, a wind control module, a humidity control module, and a sound control module. However, without limitation, the one or more environment adjustment modules can further include a variety of environment adjustment modules that can change a user's sleep environment. That is, the environment adjustment device (30) can adjust a user's sleep environment by operating the one or more environment adjustment modules based on an environment adjustment signal from the computing device (100).

According to one embodiment of the present disclosure, the computing device (100) may acquire sleep state information of a user, and may adjust the sleep environment of the user based on the sleep state information. Specifically, the computing device (100) may acquire sleep state information regarding whether the user is before, during, or after sleeping based on environment sensing information. And based on the sleep state information, the computing device (100) may adjust the sleep environment of a space where the user is located. For example, if the computing device (100) acquires sleep state information that the user is before sleeping, the computing device (100) may generate environment adjustment information related to light intensity and illuminance (e.g., 3000K white light, 30 lux illuminance), air quality (fine dust concentration, harmful gas concentration, air humidity, air temperature, etc.) to induce sleep based on the sleep state information. The computing device (100) may transmit the environment adjustment information related to light intensity and illuminance and air quality for inducing sleep to the environment adjustment device (30). In this case, the environment adjustment device (30) may adjust the light intensity and illuminance of the space in which the user is located to an appropriate intensity and illuminance for inducing sleep (e.g., 3000K white light at an illuminance of 30 lux) based on the environment adjustment information received from the computing device (100). In other words, the environment adjustment information generated by the computing device (100) may be transmitted to a lighting device, which is one embodiment of the environment adjustment device (30), to adjust illuminance, etc. in a sleep space.

In addition, the computing device (100) may generate environment adjustment information, such as removing fine dust, removing harmful gases, starting allergy care, starting deodorization/sterilization, adjusting dehumidification/humidification, adjusting blowing intensity, adjusting operation noise of the environment adjustment device (30), and various information related to LED lighting, based on the user's sleep state information.

In addition, in an embodiment such as FIG. 1 (c), for example, at least one of the electronic devices illustrated in FIG. 1 (c) may perform the operations described above.

For example, the environment adjustment information generated by the computing device (100) may be communicated to an air purifier or air conditioner, which is an embodiment of the environment adjustment device (30), to adjust temperature, humidity, or air quality in a room, vehicle, or sleep space.

In the following, the terms “sleep mode” and “wake-up mode” will be used for convenience in describing an operation of a smart home appliance. “Sleep mode” is a concept that includes an operation mode of the smart home appliance at each stage when the user is preparing to sleep, when the user is waking up, and when the user is sleeping, respectively. “Wake-up mode” is a concept that includes an operation mode of the smart home appliance in each of pre-wake-up, wake-up, and post-wake-up stages.

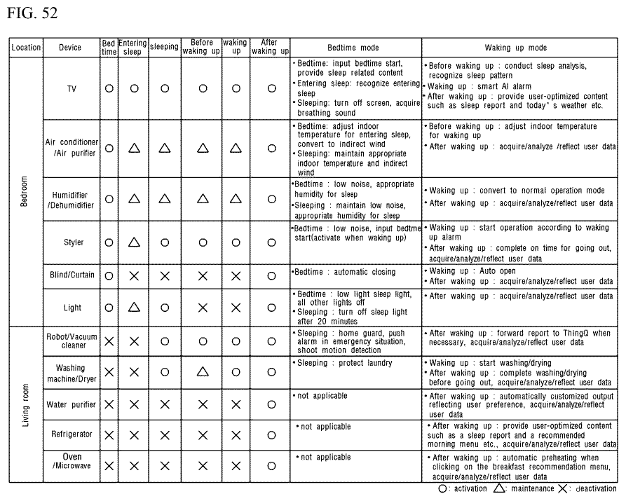

FIG. 52 is a table describing a location where the environment adjustment device is placed, whether it is activated according to sleep state information, and exemplary operations in sleep mode and wake-up mode by detailed product. Specifically, the table describes that, by location where the environment adjustment device (30) is placed and specific product of the environment adjustment device (30), whether it is activated according to the sleep state information (bedtime, entering sleep, sleeping, before waking up, waking up, after waking up), and the exemplary operations in the sleep mode and the wake mode are described. The environment adjustment information may include control information to cause the activation status, sleep mode and wake-up mode operations to be performed for each product.

The foregoing specific descriptions on the sleep state information and environment adjustment information are examples only, and the present invention is not limited thereto.

According to one embodiment of the present disclosure, the environment sensing information utilized by the computing device (100) to analyze sleep state may include information acquired in a non-invasive manner during a user's activity in a space or during sleep. As a specific example, the environment sensing information may include sounds generated by the user's tossing and turning during sleep, sound related to muscle movement, or sounds related to the user's breathing during sleep. Furthermore, the environment sensing information may include movement and distance information related to the user's movements during sleep, and breathing information generated based on the movement.

According to an embodiment, the environment sensing information may include sleep sound information. The sleep sound information may mean sound information related to movement patterns and breathing patterns that occur during the user's sleep. Furthermore, the environment sensing information may include sleep movement information, wherein the sleep movement information may mean information related to movement patterns and breathing patterns that occur during the user's sleep.

In an embodiment, the environment sensing information may be acquired via the user terminal (10) possessed by the user. For example, the environment sensing information to the user's activities in a space may be acquired through a microphone module equipped on the user terminal (10). Furthermore, the environment sensing information related to the user's activity in a space may be acquired through a radar sensor equipped on the user terminal (10).

Generally, the microphone module equipped on the user terminal (10) possessed by the user may be comprised of MEMS (Micro-Electro Mechanical Systems), as the user terminal (10) needs to be relatively small in size. Such a microphone module can be very small, but may have a low signal-to-noise ratio (SNR) compared to a condenser microphone or a dynamic microphone. A low signal-to-noise ratio may mean that the ratio of noise, which is a sound that is not to be identified, to the sound that is to be identified is high, making it difficult to identify the sound (i.e., unclear).

The environment sensing information subject to analysis in the present invention may include sound information related to a user's breathing and movement acquired during sleep, i.e., sleep sound information. Such sleep sound information is information about very small sound (i.e., sounds that are difficult to distinguish), such as the user's breathing and movement, and is acquired along with other sounds in a sleep environment. Therefore, when the sleep sound information is acquired through a microphone module such as the one described above with a low signal-to-noise ratio, detection and analysis may be difficult.

According to one embodiment of the present disclosure, the computing device (100) may acquire sleep state information based on the environment sensing information acquired from the user terminal (10). Specifically, the computing device (100) may convert and/or adjust unclearly acquired environment sensing information, including a lot of noise, into data that can be analyzed. The converted and/or adjusted data may be utilized to perform training on an artificial neural network. When pre-training of the artificial neural network is complete, the trained neural network (e.g., a sound analysis model) may acquire sleep state information of the user based on the data (e.g., spectrogram) acquired (e.g., converted and/or adjusted) in response to the sleep sound information. In embodiments, the sleep state information may include sleep stage information regarding changes in the user's sleep stage during sleep as well as information regarding whether the user is sleeping. As a specific example, the sleep state information may include sleep stage information indicating that the user was in a REM sleep at a first time point, and the user was in a light sleep at a second time point that is different from the first time point. In this case, through the sleep state information, information can be acquired that the user was in a relatively deep sleep at the first time point and was in a lighter sleep at the second time point.

In other words, when the computing device (100) acquires the sleep sound information having a low signal-to-noise ratio through a user terminal (e.g., an artificial speaker, a bedroom IoT device, a cell phone, etc.) that is commonly used to collect sound, the computing device (100) can process the sleep sound information into data suitable for analysis, and process the processed data to provide sleep state information related to changes of sleep stages. This eliminates the need for a contact-type microphone on the user's body to acquire clear sound. Furthermore, it can provide increased convenience by allowing the user to monitor his or her sleep status only with a software update in a typical home environment, without purchasing any additional devices with a high signal-to-noise ratio.

In FIG. 1 (a), the computing device (100) and the environment adjustment device (30) are depicted as separate entities. However, according to embodiments of the present disclosure, the environment adjustment device (30) may be included in the computing device (100) to perform sleep state measurement and environment adjustment operation functions in one integrated device.

In addition, in an embodiment such as FIG. 1 (c), for example, at least one of the electronic devices shown in FIG. 1 (c) may perform the operations described above.

In embodiments, the computing device (100) may be a terminal or a server, and may include any type of device. The computing device (100) may be a digital device, such as a laptop computer, notebook computer, desktop computer, web pad or mobile phone, which is a computationally capable digital device with a processor and memory. The computing device (100) may be a web server that processes services. The foregoing types of servers are only examples and the present invention is not limited thereto.

According to one embodiment of the present disclosure, the computing device (100) may be a server that provides a cloud computing service. More specifically, the computing device (100) may be a server providing a cloud computing service, which is a type of Internet-based computing in which information is processed by another computer connected to the Internet rather than a user's computer. The cloud computing service may be a service that stores data on the Internet which can be easily shared and delivered with simple operations and clicks, and is available to users anytime and anywhere through Internet access without installing any necessary materials or programs on their own computers. In addition, the cloud computing service does not just store data on servers on the Internet, but also allow users to perform tasks using functions of applications provided on the web without installing programs. It may also be a service that allows multiple people to share documents and work on it simultaneously. Furthermore, the cloud computing service may be implemented in the form of at least one of an infrastructure as a service (IaaS), PaaS (Platform as a Service), SaaS (Software as a Service), a virtual machine-based cloud server, and a container-based cloud server. In other words, the computing device (100) of the present invention may be implemented in the form of at least one of the above-described cloud computing services. The specific description of the above-described cloud computing services is by way of example only, and may include any platform for building a cloud computing environment of the present invention.

### Overall Configuration of the Computing Device

Specific configurations, technical features and effects according to the technical features of the computing device (100) of the present invention will be described with reference to the accompanying drawings.

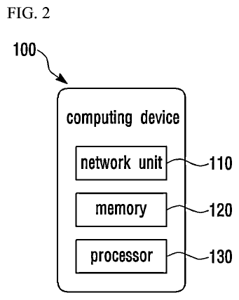

FIG. 2 depicts a block diagram of a computing device for adjusting a sleep environment based on sleep state information related to one embodiment of the present disclosure.

As shown in FIG. 2, a computing device (100) may include a network unit (110), a memory (120) and a processor (130). The present invention is not limited to the components included in the computing device (100) described above. In other words, additional components may be included or some of the foregoing components may be omitted, depending on the implementation of the embodiments of the disclosure.

According to one embodiment of the present disclosure, the computing device (100) may include the user terminal (10), the external server (20), the environment adjustment device (30) and a network unit (100) that transmits and receives data. The network unit (110) may transmit and receive to and from other computing devices, servers, etc. data for performing a sleep environment adjustment method, etc. based on sleep state information according to one embodiment of the present disclosure. In other words, the network unit (110) may provide a communication function among the computing device (100), the user terminal (10), the external server (20) and the environment adjustment device (30). For example, the network unit (110) may receive sleep test records and electronic health records for a plurality of users from a hospital server. In another example, the network unit (110) may receive environment sensing information related to a space in which the user is located from the user terminal (10). In another example, the network unit (110) may transmit environment adjustment information for adjusting the environment of the space where the user is located to the environment adjustment device (30). Further, the network unit (110) may allow information to be transmitted among the computing device (100), the user terminal (10) and the external server (20) by calling a procedure to the computing device (100).

The network unit (110) according to one embodiment of the present disclosure may use various wired communication systems, such as a public switched telephone network (PSTN), xDSL (x Digital Subscriber Line), RADSL (Rate Adaptive DSL), MDSL (Multi Rate DSL), VDSL (Very High Speed DSL), UADSL (Universal Asymmetric DSL), HDSL (High Bit Rate DSL), and local area network (LAN).

In addition, the network unit (110) presented herein may use various wireless communication systems that may be realized in the present and in the future, e.g., mobile communication systems such as 4G, 5G (LTE), and satellite communication systems such as Starlink.

In the present invention, the network unit (110) may be configured in any of its communication aspects such as wired and wireless, and may comprise various communication networks such as a personal area network (PAN), a wide area network (WAN), and the like. The network may also be the publicly available World Wide Web (WWW) and utilize wireless transmission technologies for a short-range communication such as Infrared Data Association (IrDA) or Bluetooth. The technologies described herein may be used in other networks as well as the above-mentioned networks.

According to one embodiment of the present disclosure, the memory (120) may store a computer program for performing a method of adjusting a sleep environment based on the sleep state information according to one embodiment of the present disclosure, and the stored computer program may be read and executed by the processor (130). Further, the memory (120) may store any form of information generated or determined by the processor (130) and any form of information received by the network unit (110). Furthermore, the memory (120) may store data related to the user's sleep. For example, the memory (120) may temporarily or permanently store input/output data (e.g., environment sensing information related to the user's sleep environment, sleep state information corresponding to the environment sensing information, or environment adjustment information based on the sleep state information).

According to one embodiment of the present disclosure, the memory (120) may be a storage medium of at least one of the following types: a flash memory type, a hard disk type, a multimedia card micro type, a card type of memory (e.g., SD or XD memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device (100) may also operate in connection with a web storage, which performs a storage function of the memory (120) over the Internet. The foregoing description of the memory is example only, and the present invention is not limited thereto.

The computer program, when loaded into the memory (120), may include one or more instructions that cause the processor (130) to perform methods/operations according to various embodiments of the present disclosure. That is, by executing one or more instructions, the processor (130) may perform methods/operations according to various embodiments of the present disclosure.

In one embodiment, the computer program includes: acquiring sleep state information of a user: generating environment adjustment information based on the sleep state information; and transmitting the environment adjustment information to an environment adjustment device. The computer program may include one or more instructions that cause a sleep environment adjustment method to be performed according to the sleep state information.

According to one embodiment of the present disclosure, the processor (130) may comprise one or more cores, and may include a processor for data analysis or deep learning such as a central processing unit (CPU) of a computing device, a general purpose graphics processing unit (GPGPU), a tensor processing unit (TPU), and the like.

The processor (130) may read a computer program stored in the memory (120) to perform data processing for machine learning according to one embodiment of the present disclosure. According to one embodiment of the disclosure, the processor (130) may perform operations for training a neural network. The processor (130) may perform computations for training the neural network, such as processing input data for training in deep learning (DL), extracting features from the input data, calculating errors, and updating weights of the neural network using backpropagation.

In addition, at least one of CPU, GPGPU, and TPU of the processor (130) may process training of network functions. For example, CPU and the GPGPU may together process training of the network functions and classifying data using the network functions. Further, in one embodiment of the present disclosure, the processors of a plurality of computing devices may be used together to process training of the network functions and classifying data using the network functions. Furthermore, a computer program executed on the computing device according to one embodiment of the present invention may be a CPU, GPGPU, or TPU executable program.

In the present disclosure, the network function may be used interchangeably with an artificial neural network and a neural network. In the present disclosure, the network function may comprise one or more neural networks. In this case, the output of the network function may be an ensemble of outputs of one or more neural networks.

In the present disclosure, a model may include a network function. The model may also include one or more network functions, and in this case an output of the model may be an ensemble of outputs of one or more network functions.

The processor (130) may read a computer program stored in the memory (120) to provide a sleep analysis model according to one embodiment of the present disclosure. Based on sleep state information, the processor (130) may perform calculations to produce environment adjustment information according to one embodiment of the present disclosure. According to one embodiment of the disclosure, the processor (130) may perform calculations to train a sleep analysis model. The sleep analysis model will be described in more detail below. Based on the sleep analysis model, sleep information regarding a user's sleep quality may be inferred. The environment sensing information acquired from the user in real time or periodically may be input to the sleep analysis model as an input to output data related to the user's sleep.

The training of such a sleep analysis model, and the inference based thereon, may be performed by the computing device (100) in FIG. 1 (a). In other words, both the training and the inference may be designed to be performed by the computing device (100). However, in other embodiments, the training may be performed by the computing device (100) and the inference may be performed by the user terminal (10). Furthermore, the training may be performed on the environment adjustment device (30), which may be implemented as a smart home appliance (e.g., an air conditioner, TV, lighting, refrigerator, air purifier, etc.). In another embodiment, the training and the inference may be performed by the sleep environment adjustment device (400) in FIG. 1 (b). In other words, both the training and the inference may be performed by the sleep environment adjustment device (400).

In addition, in an embodiment such as FIG. 1 (c), for example, at least one of the electronic devices depicted in FIG. 1 (c) may perform at least one of the above-described operations.

According to one embodiment of the present disclosure, the processor (130) may generally handle the overall operation of the computing device (100). The processor (130) may process signals, data, information, etc. that are input or output through the components mentioned above. Also, the processor (130) may run applications stored in the memory (120) to provide or process appropriate information or functions to the user terminal.

According to one embodiment of the present disclosure, the processor (130) may acquire sleep state information of a user. According to one embodiment of the present disclosure, acquiring the sleep state information may be acquiring or loading the sleep state information stored in the memory (120). Furthermore, acquiring the sleep state information may be receiving or loading data from another storage medium, another computing device, a separate processing module within the same computing device based on wired or wireless communication means.

In addition, in an embodiment such as FIG. 1 (c), for example, at least one of the electronics depicted in FIG. 1 (c) may perform at least one of the actions described above.

### Sleep State Information

In one embodiment, sleep state information may include information regarding whether a user is sleeping. Specifically, the sleep state information may include at least one of first sleep state information that the user is before sleep, second state information that the user is during sleep, and third sleep state information that the user is after sleep. In other words, if the first sleep state information is inferred with respect to a user, the processor (130) may determine that the user is in a pre-sleep state (i.e., before bedtime). If the second sleep state information is inferred, the processor (130) may determine that the user is in a sleeping state. If the third sleep state information is acquired, the processor (130) may determine that the user is in a post-sleep state (i.e., awake).

Such sleep state information may be characterized as being acquired based on environment sensing information. The environment sensing information may include sensing information acquired from a space in which the user is located in a non-contact manner.

According to one embodiment, the processor (130) may acquire environment sensing information. Specifically, the environment sensing information may be acquired via a user terminal (10) carried by a user. For example, the user terminal (10) carried by the user may acquire environment sensing information related to a space in which the user is located, and the processor (130) may receive the environment sensing information from the user terminal (10). The environment sensing information may be sound information acquired in a non-contact manner in the user's daily life. For example, the environment sensing information may include various sound information acquired on the user's life such as sound information related to watching television, cleaning, sound information related to cooking food, sound information related to watching television, sleep sound information acquired during sleep, etc. In embodiments, the sleep sound information acquired during the user's sleep may include sounds generated by the user's tossing and turning during sleep, sounds related to muscle movements or sounds related to the user's breathing during sleep. In other words, sleep sound information in the present invention may refer to sound information related to movement patterns and breathing patterns during the user's sleep.

### Sleep Analysis Information and Sleep Stage Information

A sleep analysis analyzes various information such as bedtime, wake-up time, total sleep time etc., and according to one embodiment, and the processor (130) may extract sleep stage information. The sleep stage information can be extracted based on a user's environment sensing information. Sleep stages may be divided into a non-REM (NREM) sleep, and a rapid eye movement (REM) sleep, and the NREM sleep may be further divided into a plurality of stages (e.g., two stages of light and deep, four stages of N1 to N4). The settings of sleep stages can be defined as common sleep stages, but they can also be arbitrarily set to various sleep stages depending on a designer's choice. By analyzing sleep stages, it is possible to predict not only sleep quality, but also sleep disorders (e.g., sleep apnea) and their underlying causes (e.g., snoring).

In a sleep analysis, changes in sleep stages can be analyzed and a hypnogram can be generated to identify changes in the analyzed sleep stages, thereby identifying the user's sleep cycle.

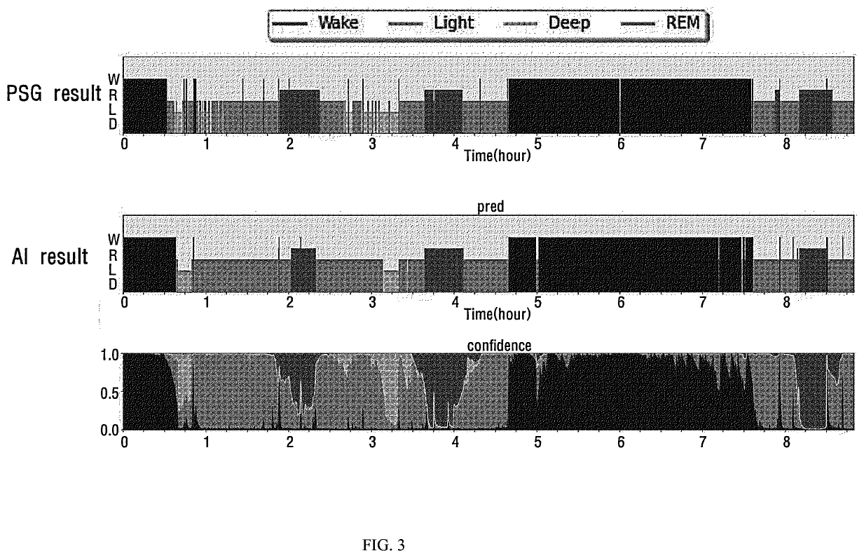

FIG. 3 is a diagram comparing a polysomnography (PSG) result with an analysis result using an AI algorithm according to the present invention.

As shown in FIG. 3, the sleep stage information acquired according to the present invention is not only highly consistent with polysomnography, but also includes more precise and meaningful information regarding sleep stages (Wake, Light, Deep, REM). The hypnogram at the bottom of FIG. 3 depicts the probability of belonging to one of the four classes (Wake, Light, Deep, REM) at 30-second intervals when predicting sleep stages based on the user's sound information. The four classes represent wakefulness, a light sleep, a deep sleep and a REM sleep, respectively.

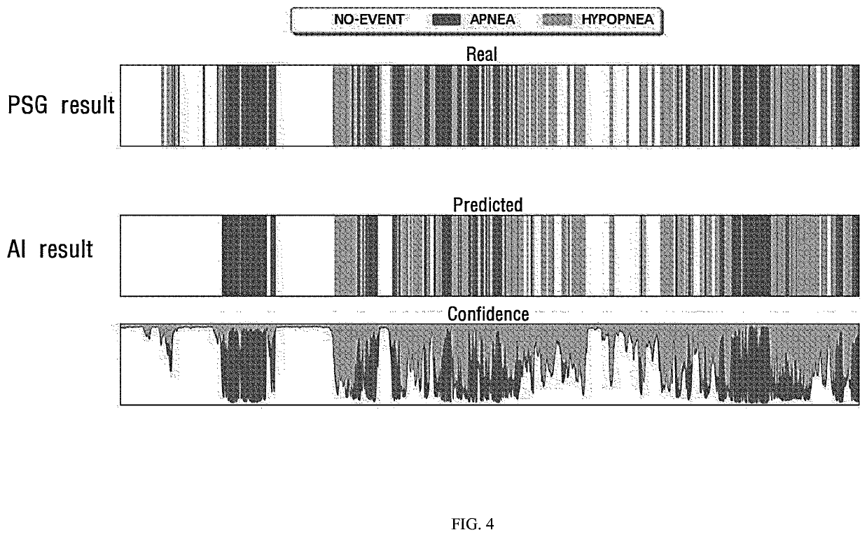

FIG. 4 is a diagram comparing a polysomnography (PSG) result with an analysis result (AI result) using an AI algorithm according to the present invention with respect to sleep apnea and hypopnea. The hypnogram shown at the bottom of FIG. 4 indicates the probability of belonging to one of two conditions (sleep apnea, hypopnea) at 30-second intervals when the user's sound information is input to predict sleep disorders.

Using the sleep stage information according to the present invention, as shown in FIG. 4, the sleep stage information acquired according to the present invention is highly consistent with the polysomnography and includes more precise analytical information regarding apnea and hypopnea.

The processor (130) may generate environment adjustment information based on sleep stage information. For example, if a sleep stage is in the Light stage or N1 stage, the environment adjustment information may be generated to control the environment adjustment device (an air conditioner, light, air purifier, etc.) to induce deep sleep.

For example, a portion of the smart home appliance (800) according to embodiments of the present disclosure may cause an alarm to sound when a REM sleep is detected within 30 minutes of a wake-up time set by a user.

This is because when an alarm rings during a REM sleep, the user may wake up more refreshed. The sleep management application according to the present invention may detect a REM sleep in real time during the user's sleep and deliver an auditory or tactile stimulus to the user to wake the user up within the time.

In addition, a portion of the smart home appliance (800) according to an embodiment of the present disclosure may detect periods of unstable breathing during the user's sleep based on sleep sound information, and provide the user with vibrotactile stimulation to induce a return to steady breathing.

In general, sustained sleep apnea is likely to lead to sympathetic nervousness and subsequent cardiovascular diseases. Therefore, when a period of breathing instability is detected during the user's sleep through the sleep management application according to the present invention, auditory and tactile stimuli can be delivered to the user through a portion of the smart home appliance (800) according to an embodiment of the present disclosure to interrupt the user's breathing instability.

Based on body movement information or posture information of the user, obstructive sleep apnea may be screened in stages.

In a sleep analysis, sleep quality, sleep stages, and sleep apnea are analyzed based on sleep sound information. The sleep sound information can refer to sound information related to breathing that occurs during the user's sleep.

The sleep analysis performs pre-processing of the user's sleep sound information and analyzes the user's sleep stages through AI algorithms, and specific analysis methods are described in more detail below.

### Identifying Singularities Based on Detecting a Preset Pattern

According to one embodiment of the present disclosure, the processor (130) may acquire sleep state information based on environment sensing information. Specifically, the processor (130) may identify a singularity where information of a preset pattern is detected in the environment sensing information. The information of the preset pattern can be related to a breathing and movement pattern related to sleep. For example, in a wake state, a breathing pattern may be irregular and body movements may be more frequent because all nervous systems are active. Also, since throat muscles are not relaxed, breathing sounds may be very low. On the other hand, when a user is sleeping, the autonomic nervous system is stabilized, breathing may become more regular, with less body movements, and breathing sounds may become louder. In other words, the processor (130) may identify as a singularity in the environment sensing information a point in time when a preset pattern of sound information related to regular breathing, less body movements, or less breathing sounds is detected. Further, the processor (130) may acquire sleep sound information based on the environment sensing information based on the identified singularity. The processor (130) may identify a singularity related to a point in time when the user is sleeping in the time-series acquired environment sensing information, and may acquire the sleep sound information based on the singularity.

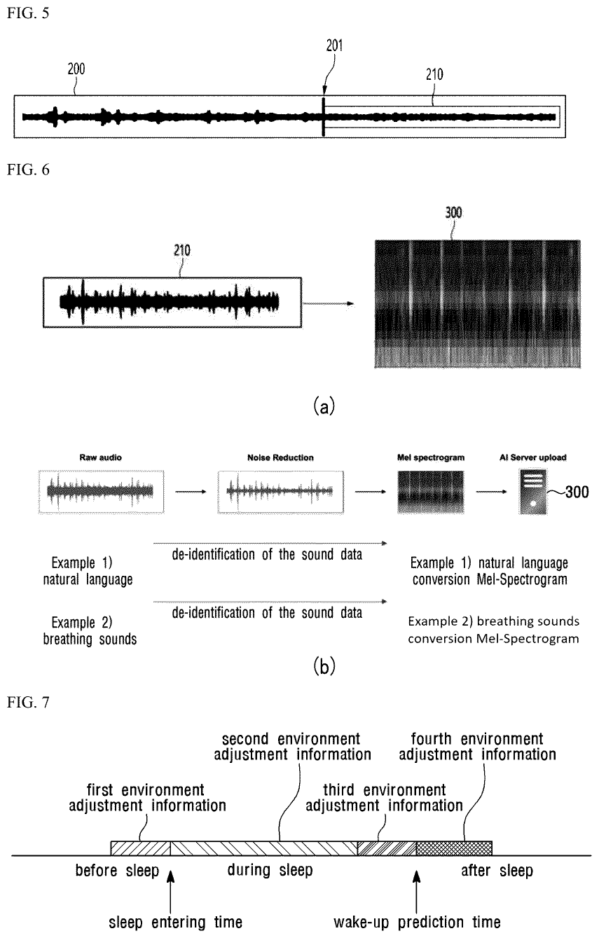

FIG. 5 is an example diagram to illustrate a process for acquiring sleep sound information (210) from environment sensing information (200) related to one embodiment of the present disclosure.

As a specific example, referring to FIG. 5, the processor (130) may identify a singularity (201) related to a time when a preset pattern is identified from environment sensing information (200). Based on the identified singularity, the processor (130) may acquire sleep sound information (210) based on sound information acquired after the singularity. The waveforms and singularities related to sounds in FIG. 5 are merely examples for understanding the present invention, and the present invention is not limited thereto.

In other words, by identifying singularities related to the user's sleep from the environment sensing information, the processor (130) may extract and acquire only the sleep sound information from the vast amount of sound information (i.e., environment sensing information) based on the singularities. This can provide convenience by automating a process of recording the user's sleep duration, while also contributing to improving accuracy of the acquired sleep sound information.

In addition, in embodiments, the processor (130) may acquire sleep state information regarding whether the user is pre-sleep or sleeping based on the identified singularity (201) from the environment sensing information (200). Specifically, the processor (130) may determine that the user is pre-sleep if the singularity (201) is not identified. And, if the singularity (201) is identified, the processor (130) may determine that the user is sleeping after the singularity (201). In addition, the processor (130) may determine that identify a time point (e.g., a wake-up time point) after the singularity (201) is identified where the preset pattern is not observed, and if that time point is identified, the processor (130) may determine that the user is after sleep, that is, has woken up.

In other words, the processor (130) may acquire sleep state information regarding whether the user is before, during, or after sleep based on whether the singularity (201) is identified in the environment sensing information (200) and whether a preset pattern is consistently detected after the singularity is identified.

Or, for example, in an embodiment such as FIG. 1 (c), at least one of the electronic devices shown in FIG. 1 (c) may perform at least one of operations described above.

In addition, the processor (830) included in the smart home appliance (800) according to one embodiment of the present disclosure may identify a singularity (201) related to a time when the preset pattern is identified from the sound information (200).

Based on the identified singularity (201), the processor (830) may acquire the sleep sound information (210) based on sound information acquired after the singularity (201).

The waveforms and singularities related to sound in FIG. 5 are merely examples for understanding the present invention, and the present invention is not limited thereto.

In other words, the processor (830) included in the smart home appliance (800) according to one embodiment of the present disclosure may identify the singularity (201) related to the user's sleep from the sound information. By doing so, only the sleep sound information (210) may be extracted and acquired from the vast amount of environment sensing information (i.e., sound information) based on the singularity (201).

This can provide convenience by allowing the user to automate the process of recording his or her sleep duration, while also contributing to improving the accuracy of the acquired sleep sound information.

In addition, in embodiments, the processor (830) may acquire sleep state information regarding whether the user is pre-sleep or sleeping based on the singularity (201) identified from environment sensing information (200). Specifically, the processor (830) may determine that the user is pre-sleep if the singularity (201) is not identified. And if the singularity (201) is identified, the processor (830) may determine that the user is sleeping after the singularity (201).

In addition, the processor (830) may identify a time point (e.g., a wake-up time point) after the singularity (201) is identified, at which the preset pattern is not observed, and if such a time point is identified, the processor (830) may determine the user is after sleep, i.e., has woken up.

In other words, the processor (830) may acquire sleep state information regarding whether the user is before, during or after sleep based on whether the singularity (201) is identified in the environment sensing information (200) and whether the preset pattern is continuously detected after the singularity is identified.

On the one hand, the processor (830) may acquire the sleep state information based on the sleep sound information rather than the environment sensing information (200).

In the present invention, the sleep sound information is used to identify the user's sleep state information in advance during the first sleep analysis, which can further improve the reliability of the analysis of the sleep state.

The sleep analysis method according to the present invention generates an inference model through deep learning of environment sensing information, and the inference model extracts the user's sleep states and sleep stages.

Briefly, environment sensing information (sound information) including sleep sound information, etc., is converted into a spectrogram, and an inference model is generated based on the spectrogram.

At this time, user privacy cannot be overlooked in a sleep analysis using sound information, and the present invention utilizes a process of performing preprocessing of environment sensing information (sound information) to protect user privacy.

As described above, an inference model for extracting the user's sleep states and sleep stages is generated through deep learning of the environment sensing information. Briefly, the environment sensing information, including sound information, etc., may be converted into a spectrogram, and an inference model may be generated based on the spectrogram.

The inference model may be built on the computing device (100) shown in FIG. 1 (a) or the sleep environment adjustment device (400) shown in FIG. 1 (b) as described above.

Then, environment sensing information, including a user's sound information, acquired through a user terminal is input to the corresponding inference model, and sleep state information and/or sleep stage information is output as results. In this case, learning and inference may be performed by the same entity, or the learning and inference may be performed by separate entities. That is, both learning and inference may be performed by the computing device (100) of FIG. 1 (a) or the environment adjustment device (400), the learning may be performed by the computing device (100), but the inference may be performed by the user terminal (10). The learning may be performed by the computing device (100), but the inference may be performed by the environment adjustment device (30) implemented as a smart home appliance (various home appliances such as air conditioner, TV, lights, refrigerator, air purifier, and the like).

In addition, in an embodiment such as FIG. 1 (c), for example, at least one of electronic devices shown in FIG. 1 (c) may perform at least one of operations described above.

### Sleep Analysis Model and Sleep Analysis Method

According to one embodiment of the present disclosure, the sleep stage information may be characterized as being acquired through a sleep analysis model that analyzes a user's sleep stages based on environment sensing information. That is, the sleep stage information of the present invention may be acquired through the sleep analysis model.

According to one embodiment of the present disclosure, the processor (130) or the processor (830) may acquire environment sensing information, and may acquire sleep sound information based on the corresponding environment sensing information. In this case, the sleep sound information may be information related to sounds acquired during the user's sleep, such as sounds generated by the user's tossing and turning during the user's sleep, sound related to muscle movements, or sound related to the user's breathing during the user's sleep.

A sleep analysis method according to an embodiment of the present disclosure will be described with the following figures.

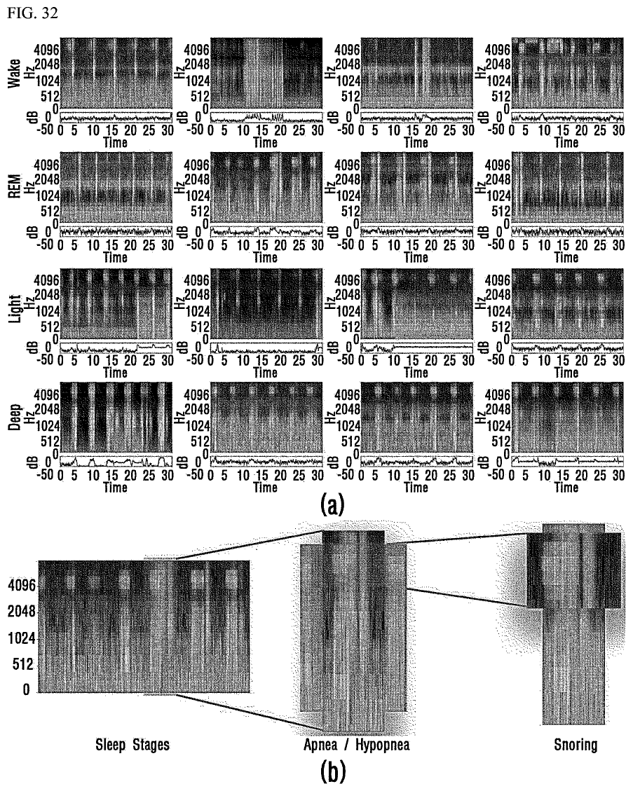

FIG. 32 (a) is a diagram to illustrate a sleep stage analysis using spectrogram in a sleep analysis method according to the present invention.

FIG. 32 (b) is a diagram to illustrate sleep disorder determination using a spectrogram in a sleep analysis method according to the present invention.

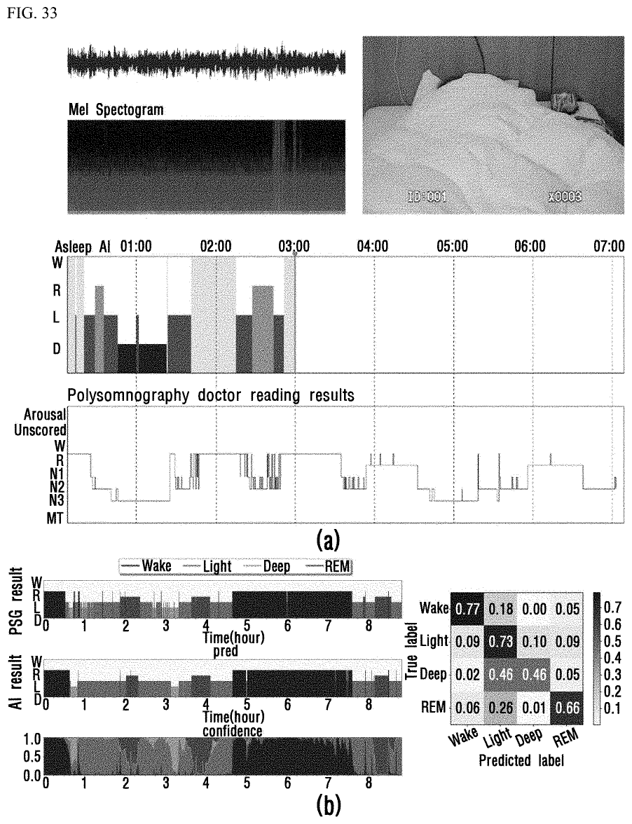

FIG. 33 (a) is a diagram to illustrate an experimental process to verify the performance of a sleep analysis method according to the present invention.

FIG. 33 (b) is a graph of the performance verification of the sleep analysis method according to the present invention, comparing the polysomnography result (PSG result) and the analysis result (AI result) using the AI algorithm according to the present invention.

As shown in FIG. 32 (a), when the user's sleep sound information is input, the corresponding sleep stage (Wake, REM, Light, Deep) may be immediately inferred.

In addition, a secondary analysis based on the sleep sound information may extract the time when sleep disorders (sleep apnea, hyperventilation) or snoring occurred through the singularity of the Mel spectrogram corresponding to the sleep stage.

As shown in FIG. 32 (b), the breathing pattern in a single Mel spectrogram may be analyzed, and if characteristics corresponding to an apnea or hyperpnea event are detected, that point in time may be determined to be the point in time when a sleep disruption occurred. Here, the process of classifying the event as snoring rather than apnea or hyperpnea through a frequency analysis may be further included.

As shown in FIG. 33 (a), the user's sleep image and sleep sound are acquired in real time, and the acquired sleep sound information is immediately converted into a spectrogram. At this time, preprocessing of the sleep sound information may be performed. The spectrogram is input to the sleep analysis model and the sleep stages are analyzed immediately.

When compared to polysomnography (PSG) results, it can be seen that the results of the sleep analysis model that uses sleep sound information as input are very accurate.

The hypnogram at the bottom of FIG. 33 (a) shows the probability of belonging to one of the four classes (Wake, Light, Deep, REM) at 30-second intervals when predicting sleep stages based on the user's sleep sound information. The four classes refer to wakefulness, light sleep, deep sleep, and REM sleep, respectively.

As shown in FIG. 33 (b), the sleep analysis results acquired according to the present invention are not only highly consistent with polysomnography, but rather includes more precise and meaningful information regarding sleep stages (Wake, Light, Deep, and REM).

### Generation and Acquisition of Spectrograms

FIG. 6 (a) is an exemplary diagram to illustrate a method for acquiring spectrograms corresponding to sleep sound information related to one embodiment of the present disclosure.

According to the present invention, a sleep analysis model may be generated using the spectrogram generated based on the sleep sound information. If the sleep sound information represented by audio data is used as it is, the amount of computation and computation time will increase dramatically because the amount of information is very large, the computation precision will decrease because it includes unwanted signals, and there may be a concern of privacy violation if all audio signals of the user are transmitted to the server. The present invention may remove noise from sleep sound information, convert it into a spectrogram (Mel spectrogram), and generate a sleep analysis model by learning the spectrogram, thereby reducing the amount of computation and the computation time, and protecting personal privacy.

According to an embodiment of the present disclosure, the processor (130) or the processor (830) may generate the spectrogram (300) in response to the sleep sound information (210), as shown in FIG. 6 (a).

Raw data (sleep sound information) which is the basis for generating the spectrogram (300) may be input. The raw data may be acquired through a user terminal or the like from a start time to an end time entered by the user, or may be acquired from a time when a user terminal operation (e.g., setting an alarm) is performed to a time corresponding to the terminal operation (e.g., the time of setting the alarm), or may be acquired by automatically selecting a time based on the user's sleep pattern. The raw data may be acquired by automatically determining the time of the user's intention to sleep based on sounds (such as user speech, breathing sounds, sounds of peripheral devices (TV, washing machine, etc.)) or changes in illumination, etc.

Although not shown in FIG. 6 (a), the process may further include performing preprocessing on the input raw data. The preprocessing may include noise reduction of the raw data. The noise reduction process removes noise (e.g., white noise) included in the raw data. The noise reduction process may be performed using algorithms such as spectral gating and spectral subtraction to remove background noise. Furthermore, in the present invention, a noise reduction algorithm based on deep learning may be used to perform the noise removal process. In other words, a noise reduction algorithm specialized for the user's breathing and respiration sounds may be used through deep learning. In particular, the present invention may generate spectrograms based only on amplitudes, excluding phases from raw data, but is not limited thereto. This not only protects privacy, but also improves processing speed by reducing data volume.

The processor (130) or processor (830) according to embodiments of the present disclosure, may perform a fast Fourier transform on the sleep sound information (210) to generate a spectrogram (300) corresponding to the sleep sound information (210).

The spectrogram (300) may be a visual representation of a sound or wave, and may include a combination of waveform and spectral features. The spectrogram (300) may show the difference in amplitude as a change along the time axis and the frequency axis as a difference in print density or a difference in display color.

The preprocessed sound raw data may be cropped into 30-second increments and converted to a Mel spectrogram. Accordingly, a 30-second Mel spectrogram may have a dimension of 20 frequency bins×1201 time steps. In the present invention, a split-cat method is utilized to transform the rectangular Mel spectrogram into a square shape so that the amount of information can be preserved.

On the one hand, the present invention may utilize a method of simulating measured breathing sounds in various home environment by adding clean breathing sounds to various noises generated in the home environment. The sounds may be added to each other because they have an additive property. However, adding original sound signals such as MP3 or PCM and converting them into Mel spectrogram may consume a lot of computing resources.

Therefore, the present invention presents a method for adding breathing and noise by converting each into a Mel spectrogram. By doing so, it is possible to simulate breathing sounds measured in various home environments and utilize them for training a deep learning model to secure robustness in various home environment.

In the present invention, the sleep sound information (210) is the sound related to breathing and body movements acquired during a user's sleep period, and thus may be very small sounds. Accordingly, the processor (130) or the processor (830) may convert the sleep sound information into spectrogram (300) to perform analysis on the sound. In this case, the spectrogram (300) includes information showing how the frequency spectrum of the sound changes over time, as described above. Thus, breathing or movement patterns related to relatively small sounds can be easily identified, improving the efficiency of the analysis.

In addition, in an embodiment such as FIG. 1 (c), for example, at least one of the electronics illustrated in FIG. 1 (c) may perform the operations described above.

According to one embodiment, the spectrogram may be configured to have different concentrations of frequency spectra for different sleep stages. Specifically, based on changes in energy levels of the sleep sound information alone, it may be difficult to predict whether a state is at least one of wakefulness, REM sleep, light sleep, and deep sleep. However, by converting the sleep sound information into a spectrogram, changes in the spectrum of each frequency can be readily detected. Thus, analysis in response to small sounds (e.g., breathing and body movements) may become possible.

Further, the processor (130) or processor (830) may process spectrogram (300) as input to a sleep analysis to acquire sleep stage information, A sleep analysis model is a model for acquiring sleep stage information related to changes user's sleep stages, and may output sleep stage information using the sleep sound information acquired during the user's sleep as input. In embodiments, the sleep analysis model may include a neural network model constructed via one or more network functions.

### Network Functions and Neural Networks

In embodiments of the present disclosure, a sleep analysis model may include a neural network model comprising one or more network functions. The sleep analysis model may comprise one or more network functions, wherein the one or more network functions may comprise a set of interconnected computational units that may be generally referred to as ‘nodes’. These ‘nodes’ may also be referred to as ‘neurons’. One or more network functions comprise at least one or more nodes. The nodes (or neurons) comprising the one or more network functions may be interconnected by one or more ‘links’.

FIG. 9 is a schematic diagram of one or more network functions related to one embodiment of the present disclosure.

A deep neural network (DNN) may refer to a neural network that includes a plurality of hidden layers in addition to an input layer and an output layer. Deep neural network can be used to identify latent structures in data.

In other words, it is able to identify the latent structure of photo, text, video, voice, and music (e.g., what objects are in a photo, what is the content and emotion of the text, what is the content and emotion of the voice etc.) Deep neural network may include convolutional neural network (CNN), recurrent neural network (RNN), auto encoder, generative adversarial networks (GAN), restricted Boltzmann machine (RBM), deep belief network (DBN), Q network, U network, Siamese network, etc. The foregoing description of deep neural network is by way of example only, and the present invention is not limited thereto.

In embodiments of the disclosure, the network function may include an auto encoder. An auto encoder may be a type of artificial neural network for outputting output data similar to input data. The auto encoder may include at least one hidden layer, and an odd number of hidden layers may be disposed between the input and output layers.

The number of nodes in each layer may be reduced from the number of nodes in the input layer to an intermediate layer called the bottleneck layer (encoding), and then expanded symmetrically from the bottleneck layer to the output layer (symmetrical to the input layer). The nodes in the dimensionality reduction layer and the dimensionality restoration layer may be symmetric or asymmetric.

The auto encoder according to embodiments of the present disclosure may perform nonlinear dimensionality reduction. The number of input layers and output layers may correspond to the number of sensors remaining after preprocessing of the input data. The auto-encoder structure may have a structure in which the number of nodes in the hidden layer included in the encoder decreases with distance from the input layer.

The number of nodes in the bottleneck layer (the layer with the fewest nodes between the encoder and decoder) may be kept above a certain number (e.g., more than half of the input layer) because too few nodes may not convey a sufficient amount of information.

The neural network may be trained in at least one of the following ways: supervised learning, unsupervised learning and semi-supervised learning. The learning of the neural network is intended to minimize the error in the output.

In the learning of the neural network, it repeatedly inputs learning data into the neural network and calculates the output of the neural network and the error of the target for the learning data. Then, in order to reduce the error, the error of the neural network is back propagated from the output layer of the neural network to the input layer, and the weight of each node of the neural network is updated.

For supervised learning, the learning data is used where each learning data is labeled with the correct answer (i.e., labeled learning data). For unsupervised learning, the learning data may not be labeled with the correct answer. In other words, for example, the learning data for supervised learning on data classification may be data where each learning data is labeled with a category.

The labeled training data may be input to a neural network, and an error may be calculated by comparing the output of the neural network (categories) to the labels of the learning data. In another example, for unsupervised learning for data classification, the error can be calculated by comparing the input learning data to the neural network output.

The calculated error may be back propagated in the neural network in the reverse direction (i.e., from the output layer to the input layer), and the connection weights of each node in each layer may be updated based on the backpropagation. The amount of change in the connection weights of each updated node may be determined by the learning rate.

The computation of the neural network on the input data and the backpropagation of errors may comprise a leaning cycle (epoch). The learning rate can be applied differently depending on the number of repetitions of the neural network's learning cycle.

For example, in the early stages of a neural network's learning, a high learning rate is used to increase efficiency by allowing the neural network to quickly achieve a certain level of performance. Later in the learning of a neural network, a low learning rate may be used to increase accuracy.

In the learning of a neural network, the training data can typically be a subset of real data (i.e., the data to be processed using the trained neural network), so there may be training cycles with decreasing error on the learning data but increasing error on the real data.

Overfitting is the phenomenon of overtraining on learning data, resulting in increased error on the real data. For example, a neural network that has seen a yellow cat and learned to recognize it as a cat may not recognize a cat when it sees a non-yellow cat.

Overfitting can lead to increased errors in machine learning algorithms. To prevent overfitting, various optimization methods may be used. To prevent overfitting, methods such as increasing the learning data, regularization, and dropout, where some nodes in the network are omitted from the learning process, may be applied.

Throughout this specification, the terms computational model, neural network, network function, and neural network may be used interchangeably (collectively referred to herein as a neural network). A data structure may comprise a neural network.

The data structure including the neural network may be stored on a computer-readable medium. The data structure including the neural network may also include data input to the neural network, weights of the neural network, hyper-parameters of the neural network, data acquired from the neural network, an activity function related to each node or layer of the neural network, and a loss function for learning the neural network.

The data structure including the neural network may include components of any of the above-disclosed configurations. In other words, the data structure including the neural network may comprise all or any combination of the following: data input to the neural network, weights of the neural network, hyper-parameters of the neural network, data acquired form the neural network, an activity function related to each node or layer of the neural network, and a loss function for training the neural network. In addition to the foregoing configurations, a data structure including a neural network any other information that determines characteristics of the neural network.

Further, the data structures may include all forms of data used or generated in the computational process of the neural network, and are not limited to the foregoing. The computer-readable medium may include a computer-readable recording medium and/or a computer-readable transmission medium. A neural network may comprise a set of interconnected computational units that may be generally referred to as nodes. These nodes may also be referred to as neurons. A neural network comprises at least one or more nodes.

Within a neural network, one or more nodes connected via links can form a relative relationship of input nodes and output nodes. The concepts of input node and output node are relative, and any node that is in an output node relationship with respect to one node may be in an input node relationship with respect to another node, and vice versa.

In an input node and output node relationship connected via a link, the output node may have its value determined based on data input to the input node. The nodes interconnecting the input and output nodes may have weights.

The weights may be variable and may be changed by the user or algorithm in order for the neural network to perform the desired function. For example, if one or more input nodes are interconnected to an output node by respective links, the output node may determine the output node value based on the values entered in the input nodes connected to the output node and the weights set in the links corresponding to each of the input nodes.

As described above, a neural network may include one or more nodes interconnected via one or more links to form input node and output node relationships within the neural network. Depending on the number of nodes and links in the neural network, the relationships between the nodes and links and the values of the weights assigned to each of the links, the characteristics of the neural network may be determined.

For example, if two neural networks exist with the same number of nodes and links and different weight values between the links, the two neural networks may be recognized as different.

Some of the nodes in a neural network may comprise a layer, based on their distance from the initial input node. For example, a set of nodes with a distance of n from the initial input node may comprise n layers.

The distance from the initial input node may be defined by the minimum number of links that must be crossed to reach the node from the initial input node.

However, this definition of layers is arbitrary for illustrative purposes, and the order of layers within a neural network may be defined in different ways from those described above. For example, a layer of nodes may be defined by their distance from the final output node.

An initial input node may refer to one or more nodes in the neural network from which data is input directly, without going through any links in the relationship with other nodes. Or, within a neural network, in a relationship between nodes based on links, the initial input nodes may be nodes that do not have other input nodes connected by links.

Similarly, in relation to other nodes, a final output node can refer to one or more nodes in a neural network which do not have an output node. Further, hidden nodes may refer to nodes that comprise the neural network which are not initial input nodes and final output nodes. A neural network according to one embodiment of the present disclosure may have more nodes in the input layer than nodes in the hidden layer that are closer to the output layer. A neural network according to one embodiment of the disclosure may be a form of neural network with a decreasing number of nodes as it goes from the input layer to the hidden layer.

The neural network may include one or more hidden layers. The hidden nodes in a hidden layer may take as input the outputs of the previous layer and the outputs of neighboring hidden nodes. The number of hidden nodes in each hidden layer may be same or different.

The number of nodes in the input layer may be determined based on the number of data fields in the input data and may be the same or different from the number of hidden nodes. The input data entered into the input layer may be computed by the hidden nodes of hidden layer. The input data input to the input layer may be output by the output layer, the fully connected layer (FCL).

### Feature Extraction Models and Feature Classification Models

According to one embodiment of the present disclosure, the sleep analysis model may include a feature extraction model for extracting one or more features per predetermined epoch, and a feature classification model for generating sleep stage information by classifying each of the features extracted by the feature extraction model into one or more sleep stages.

According to an embodiment, the feature extraction model may analyze the time series frequency pattern of the spectrogram (300) to extract features to extract features related to breathing sounds and breathing patterns.

In one embodiment, the feature extraction model may be configured as part of a neural network model (e.g., an auto encoder) that is pre-trained via a training data set. The training data set may comprise a plurality of spectrograms and a plurality of sleep stage information corresponding to each spectrogram.

In one embodiment, the feature extraction model may be configured via an independent deep learning model (e.g., an auto encoder) learned from the training data set. The feature extraction model may be learned via supervised or unsupervised learning methods. The feature extraction model may be learned to produce output data that is similar to the input data via the training data set.

To be more specific, during the encoding process via the encoder, only the key feature data (or features) of the input spectrogram may be learned via the hidden layer and the rest of the information may be lost. In this case, during decoding via a decoder, the output data of the hidden layer may be an approximation of the input data (i.e., the spectrogram) rather than a perfect copy. In other words, the auto encoder may be trained to adjust the weights so that the output and input data are as close as possible.

Each of the plurality of spectrograms in the training data set may be tagged with sleep stage information. Each of the plurality of spectrograms may be input to the feature extraction model, and the output corresponding to each spectrogram may be stored by matching the tagged sleep stage information.

Specifically, if a first set of training data (i.e., a plurality of spectrograms) tagged with a first sleep stage information (e.g., light sleep) is input, the features related to outputs corresponding to that input may be stored in matching with the first sleep stage information. In embodiments, the one or more features related to the output may be represented in a vector space.

In this case, the feature data output for each of the first training data sets are output from the spectrograms related to the first sleep stage, so they may be located relatively close together in vector space. In other words, multiple spectrograms may be trained to output similar features for each sleep stage.

In the case of the encoder, it may be trained to extract features that enable the decoder to recover the input data well. Thus, the feature extraction model, as implemented by the encoder among the trained auto encoder, may extract features (i.e., a plurality of features) that enable the encoder to recover the input data (i.e., the spectrogram) well.

Through the foregoing learning process, the encoder that comprises a feature extraction model may extract features corresponding to the spectrogram (e.g., a transformed spectrogram corresponding to sleep sound information) when the spectrogram is input.

In embodiments, the processor (130) or the processor (830) may process the spectrogram (300) generated in response to the sleep sound information (210) as input to a feature extraction model to extract features. Here, the sleep sound information (210) is a time series data that is acquired time series during the user's sleep. Accordingly, the processor (130) or the processor (830) may divide spectrogram (300) into predetermined epochs.

For example, the processor (130) or the processor (830) may split the spectrogram (300) corresponding to the sleep sound information (210) into 30-second increments to acquire a plurality of spectrograms. For example, if the sleep sound information is acquired for seven hours (i.e., 420 minutes) of sleep for the user, the processor (130) or the processor (830) may divide the spectrogram into 30-second increments to obtain 840 spectrograms.

Alternatively, in an embodiment such as FIG. 1 (c), at least one of the electronics shown in FIG. 1 (c) may perform at least one of the actions described above. The foregoing specific numerical descriptions of sleeping hours, time units of divisions of the spectrogram and number of divisions are by way of example only, and the present invention is not limited thereto.

The processor (130) or the processor (830) according to embodiments of the present disclosure, may process each of the plurality of segmented spectrograms as input for a feature extraction model to extract a plurality of features corresponding to each of the plurality of spectrograms. For example, if the number of the plurality of spectrograms is 840, the number of the plurality of features extracted by the feature extraction may correspondingly be 840. The foregoing specific numerical descriptions of the number of spectrograms and the number of features are examples only, and the present invention is not limited thereto.

In addition, the processor (130) or the processor (830) may process the plurality of features output by the feature extraction as input to a feature prediction model to acquire sleep stage information. In embodiments, the feature classification model may be a neural network model to predict sleep stages in response to the features.

For example, the feature classification model may comprise a fully connected layer and may be a model that classifies the feature into at least one of the sleep stages. For example, given a first feature corresponding to a first spectrogram as an input, the feature classification model may classify the first feature as shallow water.

The classification model may perform multi-epoch classification, which involves taking spectrograms related to multiple epochs as input and predicting the sleep stage of the multiple epochs. The multi-epoch classification may be intended to estimate multiple sleep stages (e.g., changes in sleep stages over time) at once using spectrograms corresponding to multiple epochs (e.g., a combination of spectrograms each representing 30 seconds) as input, rather than providing a single sleep stage analysis in response to a spectrogram of a single epoch (i.e., one spectrogram corresponding to 30 seconds).

For example, because breathing patterns change slowly compared to brainwave signs or other vital signs, accurate sleep stage estimation may be possible by observing how the pattern changes at past and future points in time. As a specific example, a subject model may take 40 spectrograms (e.g., 40 spectrograms each corresponding to 30 seconds) as input and make a prediction on the centered 20 spectrograms. In other words, by looking at all of the spectrograms from 1 to 40, the sleep stages may be predicted by classifying the spectrograms corresponding to 10 to 20. The specific numerical description of the number of spectrograms described above is by way of example only, and the present invention is not limited thereto.

In other words, in the process of estimating sleep stages, rather than performing a sleep stage prediction in response to a single spectrogram, the output may be more accurate by utilizing spectrograms corresponding to multiple epochs as input, taking into account both past and future information.

As described above, the processor (130) or the processor (830) may acquire a spectrogram based on the sleep sound information. In this case, the conversion to a spectrogram may be to facilitate analysis of breathing or movement patterns related to relatively small sounds. Further, the processor (130) or the processor (830) may utilize a sleep analysis model comprising a feature extraction model and a feature classification model to generate sleep stage information based on the acquired spectrogram. In this case, the sleep analysis model may utilize spectrograms corresponding to a plurality of epochs as inputs to perform sleep stage predictions so that both past and future information may be considered. Therefore, more accurate sleep stage information may be output.

In other words, the processor (130) or the processor (830) may utilize a sleep analysis model as described above to output sleep stage information corresponding to the sleep sound information.

Or, for example, in an embodiment such as FIG. 1 (c), at least one of electronic devices shown in FIG. 1 (c) may perform the operations described above.

According to embodiments, the sleep stage information may be information about changing sleep stages during a user's sleep. For example, the sleep stage information may refer to information that the user's sleep has changed to light sleep, normal sleep, deep sleep, or REM sleep at each time point during the user's last eight hours of sleep. The above specific description of sleep stage information by way of example only, and the present invention is not limited thereto.

### User Privacy Protection Method

FIG. 6 (b) is a conceptual diagram to illustrate a privacy protection method using Mel spectrogram transformation for sleep sound information extracted from a user in sleep analysis method according to the present invention.

As shown in FIG. 6 (b), the sound information extracted from the user, or the raw data extracted from it, the sleep sound information undergoes a preprocessing process of a noise reduction. In the noise reduction process, noise (e.g., white noise) included in the raw data is removed.

The noise reduction process may be performed using algorithms such as spectral gating and spectral subtraction to remove background noise.

Further, the present invention may utilize a deep learning-based noise reduction algorithm to perform the noise reduction process. The deep learning-based noise reduction algorithm may be specific to the user's breathing or respiration sounds, in other words, the noise reduction algorithm may be learned from the user's breathing or respiration.

The de-noised raw data is then generated as a Mel-Spectrogram. Here, a Mel-Spectrogram is a row of simplified vectors in the frequency domain for a given input sentence (text).

In this case, it is possible to utilize a method that generates Mel-Spectrogram based only on amplitudes, excluding phases from the raw data. This not only protects privacy, but also improves processing speed by reducing data size. However, in other embodiments, it is possible to generate a Mel-Spectrogram using both phases and amplitudes.

The present invention generates a sleep analysis model using a Mel-Spectrogram (300) generated based on the sleep sound information (210). If the sleep sound information expressed as audio data is used as it is, the amount of computation and computation time increase dramatically because the amount of information is very large. Furthermore, since it includes unwanted signals, the computational precision is reduced, and there is a risk of privacy violation if all audio signals of the user are transmitted to the external server (20) or the AI server (310).

The present invention removes the noise of the sleep sound information by the method described above, converts it into a Mel spectrogram, and generates a sleep analysis model by learning the Mel-Spectrogram. Because of this, the amount of computation and computation time may be reduced, and personal privacy may be protected.

In this case, de-identification of the sound data may be done for natural language and breathing sounds. This can be converted into a natural language conversion Mel-Spectrogram and a breathing sound conversion Mel-Spectrogram, respectively. In the sleep analysis according to the present invention, only the information required for the analysis model may be utilized to improve the computation speed and reduce the computation load.

### Accuracy Verification and Embodiment of the Sleep Analysis Method

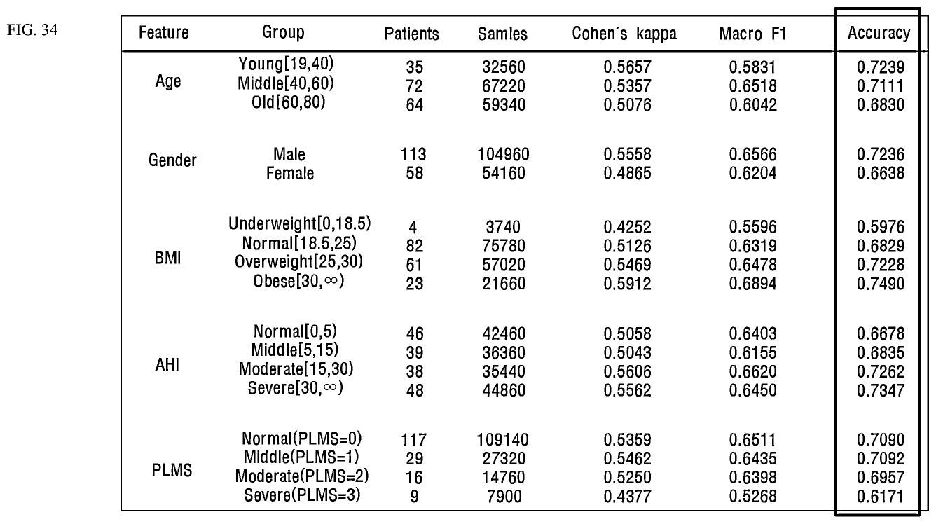

FIG. 34 is a table that verifies the accuracy of the sleep analysis method, and the data of the experimental results analyzed by age, gender, BMI, and disease status.

FIG. 34 is a conceptual diagram of one embodiment of the sleep analysis method according to the present disclosure, which illustrates the case of using a smart speaker and a smartphone for easy understanding.

Unlike the polysomnography method is hospitals, the sleep analysis method according to the present invention may turn on/off the light during the test and freely adjust the room temperature and humidity.

In other words, it is possible to verify various actual situations for ultra-gaps beyond the fixed hospital environment verification, and it is possible to conveniently and flexibly analyze sleep in various environments other than hospitals using only smart home appliances (800) and smart phones (900).

As a result, as shown in FIG. 34, the experimental results show consistently high accuracy across a wide range of age, gender, BMI, sleep apnea, and limb movement disorder subjects.

As shown in FIG. 34, for ease of understanding, the smart home appliance (800) is envisioned as a smart speaker, but is not limited thereto. In other words, the smart home appliance (800) may be implemented as a tablet personal computer, a mobile phone, a video phone, an e-book reader, a desktop personal computer, a laptop personal computer, a netbook computer, a workstation, a server, a personal digital assistant (PDA), a portable multimedia player (PMP), an MP3 player, a mobile medical device, a camera, a wearable device (e.g., smart glasses, a head-mounted device (HMD), electronic apparel, electronic bracelet, electronic necklace, electronic accessory, electronic tattoo, smart watch), smart mirror, kiosk or the like.

Further, the smart home appliance (800) may be implemented as a smart home appliance such as a television, digital video disk (DVD) player, audio, refrigerator, air conditioner, vacuum cleaner, oven, microwave, washing machine, air purifier, set-top box, home automation control panel, security control panel, TV box, game console, electronic dictionary, electronic key, camcorder or electronic picture frame, various medical devices, domestic robots, internet of things devices (e.g., light bulbs, various sensors, electrical or gas meters, sprinkler systems, smoke alarms, thermostats, toasters, exercise equipment, hot water tanks, heaters, boilers, and the like).

In addition, the smart home appliance (800) may be implemented as a piece of furniture or part of a building/structure, an electronic board, an electronic signature, a projector, or the like, and may be one or more combinations of the various devices mentioned above.

In addition, in an embodiment such as FIG. 1 (c), for example, at least one of the electronics shown in FIG. 1 (c) may correspond to one or more combinations of the various devices described above.

Therefore, the sleep analysis method according to the present invention may conveniently and simply enable in-depth sleep analysis of a user through a smart home appliance (800), such as a smartphone (900) or a smart speaker etc., regardless of time and place, even outside of a hospital.

### Configuration of the Contactless Sleep Analysis System

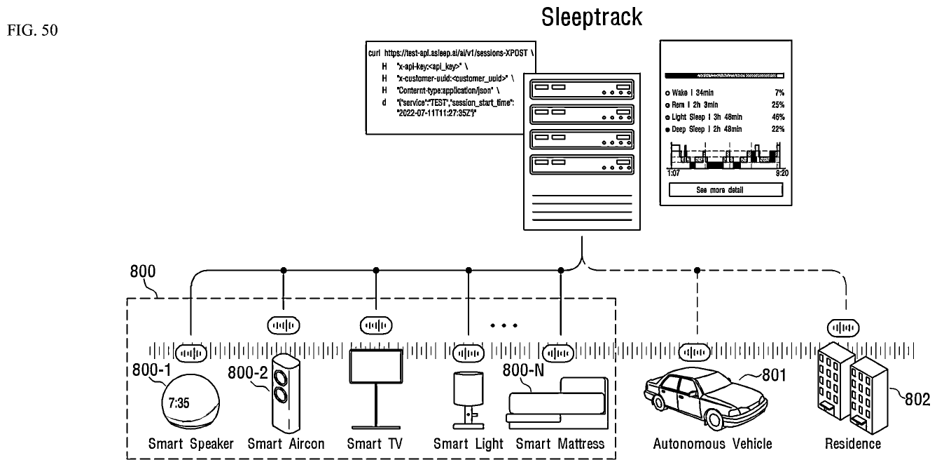

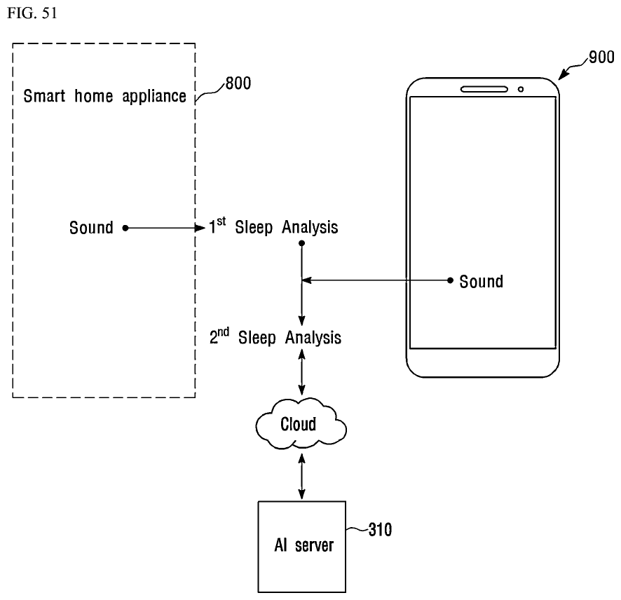

FIG. 50 is a block diagram to illustrate the operation of an AI-based contactless sleep analysis system according to the present disclosure, including one or more smart home appliances (800), a sleep track application, an autonomous vehicle (801), and a living space (802).

FIG. 51 is a block diagram to illustrate the operation of components of an AI-based contactless sleep analysis system according to the present disclosure, including a smart home appliance (800), a smartphone (900), and an AI server (310).

As shown in FIG. 50, the smart home appliance (800) according to the present disclosure has a built-in microphone to acquire sleep sound information of the user. By performing sleep analysis (contactless sleep analysis) using the sleep sound information, a more universal and precise sleep analysis may be performed.

In other words, it is possible to perform various real-world verifications beyond environment verifications such as polysomnography in hospitals. It accurately detects not only insomnia but also sleep apnea and hypopnea events in real time. It can provide sleep diagnosis solutions for a wide range of ages, genders, races, BMIs and medical conditions.

As shown in FIG. 51, the smart home appliance (800) and the smartphone (900) work together to perform a user's sleep analysis. The smart home appliance (800) and the smartphone (900) may be paired via Bluetooth or other wireless communication method.

The smartphone (900) may perform sleep analysis based on the user's sleep sound information acquired from the smart home appliance (800).

In this case, the user's sleep sound information may be acquired from the smart home appliance (800) and transmitted to the smartphone (900), but it may also be self-acquired through a microphone embedded in the smartphone (900).

In other words, in the embodiment shown in FIG. 51, the sleep stage analysis is a contactless sleep stage analysis via the smart home appliance (800) and the smartphone (900). The user can view the sleep stage analysis results derived from the smartphone (900) on the screen of the smartphone (900).

As such, even when the user is not wearing the smart home appliance (800), it is necessary for the smart home appliance (800) to be appropriately disposed around the user to receive at least a portion of the input signal for the sleep analysis (e.g., body movement information) or the input signals for the sleep analysis (sleep sound information).

In particular, in order to extract body movement information, it may be advantageous to be positioned in an area where the user's movement may at least be detected (e.g., under the pillow, on top of the mattress, etc.).

On the other hand, since sound is transmitted in a radial direction, using only sleep sound information has the advantage that the information may be collected and analyzed regardless of the user's location, distance or angle between the user and the smart home appliance (800).

Therefore, when the smart home appliance (800) of the present invention is not necessarily worn on the user, if is appropriately placed within a designated radius (e.g., 4 to 5 meters) within the user's sleep space, regardless of the user's location, distance or angle to the user, the sleep stage analysis described above is possible. The specific numerical description of the radius is for example only, and the invention is not limited thereto.

In one embodiment, the smart home appliance (800) may transmit a designated signal to allow the user to position the smart home appliance (800) closer to the user when the smart home appliance (800) is not worn by the user in order to receive input signals (sleep sound information) for sleep analysis. The designated signal may be a vibration, alarm, text, LED, or the like.

The radius between the user and the smart home appliance (800) may be derived by the smart home appliance (800), or it may be derived by the smartphone (900).

In other words, since the user's sleeping space is fixed, the location of the smart appliance (800) may be tracked to determine if the smart home appliance (800) is positioned in an appropriate location.

On the one hand, the smart home appliance (800) may be a sleep product (device) that is utilized while the user is sleeping, rather than a device that may be worn by the user.

For example, a smart speaker may be utilized as one of the smart home appliances (800). The smart speaker may include a sound sensor to measure inside to measure various sound information.

The smart speaker may use the sound information acquired through the sound sensor to perform a first sleep analysis. The smart speaker may be paired with the smartphone (900), and the information measured by the smart speaker, or the results of the first sleep analysis analyzed by the smart speaker, may be transmitted to the smartphone (900). The smart speaker may include a communication module.

In addition, as one of the smart home appliances (800), a smart mattress may be utilized. The smart mattress may include sound sensors inside to measure various sound information.

The smart mattress may use the sound information to perform a first sleep analysis. The smart mattress may be paired with the smartphone (900) to transmit the information measured by the smart mattress, or the results of the first sleep analysis analyzed by the smart mattress to the smartphone (900). In this case, the smart mattress may include a communication module.

On the one hand, the smart mattress may include various modules (temperature control module, infrared irradiation module, cooling module) for adjusting the temperature, and the temperature may be adjusted based on the results of the final sleep stage analysis. This improves the quality of the user's sleep.

On the one hand, the above-mentioned smart speaker or smart mattress may include a vibration module or an alarm module to alleviate and improve the sleep disorders described above. In other words, when sleep apnea, snoring, sleep hyperpnea, REM sleep, etc. are detected, the vibration module or alarm module of the smart speaker or smart mattress may be activated to deliver tactile or sound stimuli to the user.

In other instances, in an autonomous vehicle (801), or in a recently constructed living space (802), one or more smart devices may work in conjunction with the sleep track application to build and operate an AI-based contactless sleep analysis system according to the present disclosure.

In addition, in an embodiment such as FIG. 1 (c), for example, at least one of the electronic devices shown in FIG. 1 (c) may perform at least one of operations described above.

The foregoing description of types of smart appliances or spaces is by way of example only, and the present invention is not limited thereto.

### Smart Home Appliances in the Sleep Analysis System

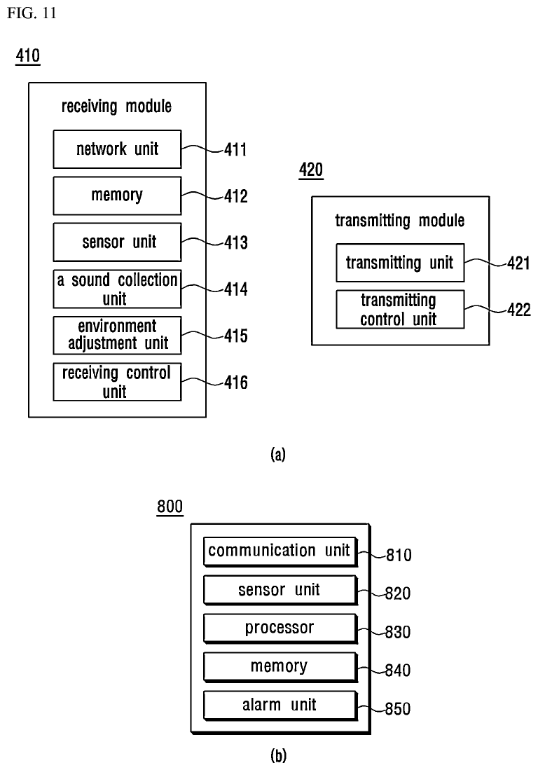

FIG. 11 (b) is a block diagram illustrating a configuration of a smart home appliance in an AI-based contactless sleep analysis system according to the present invention.

The smart home appliance (800) according to the present invention includes a communication unit (810), a sensor unit (820), a processor (830), a memory (840), and an alarm unit (850). Various configurations may be further included to perform the functions of the smart home appliance (800).

In other words, additional configurations may be included depending on the implementation of the embodiments of the present disclosure. Alternatively, some of the above configurations may be omitted, or two or more configurations may be combined into one configuration.

The communication unit (810) performs data transmission and reception with the smartphone (900) or AI server (310) via a wireless communication network. The wireless communication network may include a short-range wireless network such as Z-wave, zigbee, Wi-Fi, Bluetooth (BLE), LTE-M, LoRa (Long Range), Narrowband Internet of Things (NB-IoT), or Infrared Data Association (IrDA), etc. In addition, wireless communication networks may include 2G cellular networks such as wireless LAN (WLAN), wireless broadband (Wibro), wireless fidelity (Wi-Fi), WiMax (world interoperability for microwave access), GSM (global system for mobile communication) or CDMA (code division multiple access), 3G cellular networks such as WCDMA (wideband code division multiple access) or CDMA2000, 3.5G cellular network such as HSDPA (high speed uplink packet access) or HSUPA (high speed uplink packet access), or 4G, 5G, 6G such as LTE (long term evolution) networks, or LTE-Advanced networks, but it is not limited thereto.

The sensor unit (820) may include a microphone module for extracting sleep sound information of the user. The microphone module may comprise micro-electro mechanical systems (MEMS) for application in small devices. Such microphone modules may be manufactured to be very small and may have a very low signal noise ratio (SNR) compared to a condenser microphone or a dynamic microphone.

In this case, the sleep sound information is the information of sound signals during sleep, which closely interacts with sleep itself, and may be acquired without wearing a wearable device such as a smart watch or a smart ring.

The sensor unit (820) may include a pressure sensor, a grip sensor, a color sensor, an infrared (IR) sensor, a temperature sensor, a humidity sensor, and an illumination sensor.

The memory (840) may store a computer program for performing the sleep analysis, and the stored computer program may be read and executed by the processor (830), as described later. Furthermore, the memory (840) may store any form of information generated or determined by the processor (830) and any form of information received by the communication unit (810). In addition, the memory (840) may store data related to a user's sleep.

For example, the memory (840) may store incoming and outgoing data temporarily or permanently.

The memory (840) may be a flash memory type, a hard disk type, a multimedia card micro type, a card type of memory (such as SD or XD memory), a random access memory (RAM), static random access memory (SRAM), Read-Only Memory (ROM), Electrically Erasable Programmable Read-Only Memory (EEPROM), Programmable Read-Only Memory (PROM), magnetic memory, magnetic disk, or optical disk, and may be implemented in at least one type of storage medium, but not limited thereto.

The computer program, when loaded into the memory (840), may include one or more instructions that cause the processor (830) to perform methods/actions according to various embodiments of the present disclosure. In other words, the processor (830) may perform methods/actions according to various embodiments of the present disclosure by executing the one or more instructions.

The processor (830) may comprise one or more cores and may include a processor for data analysis such as a central processing unit (CPU), a general purpose graphics processing unit (GPGPU), a tensor processing unit (TPU), etc. of the smart home appliance and deep learning.

The processor (830) may read computer programs stored in the memory (840) to perform data processing for machine learning according to one embodiment. The processor (830) may perform computations for training a neural network, according to one embodiment of the present disclosure.

The processor (830) may perform computations for training a neural network such as processing input data for training in deep learning (DL), extracting features from the input data, calculating errors, and updating weights of the neural network using backpropagation.

In addition, at least one of the CPU, GPGPU and TPU of processor (830) may process training of the network functions.

For example, the CPU and the GPGPU may together process training of a network function and classifying data using the network function. Also, in one embodiment of the present disclosure, a processor of a plurality of smart home appliances may be used together to process the learning of the network function and the classification of data using the network function.

Further, a computer program executed on a smart home appliance according to one embodiment of the present disclosure may be a CPU, GPGPU or TPU executable program.

The network function may be used interchangeably with an artificial network and a neural network. A network function may comprise one or more neural networks. In this case, the output of the network function may be an ensemble of the outputs of the one or more neural networks.

A model (inference model) may include network functions. A model may also include one or more network functions. In this case, the output of the model may be an ensemble of the outputs of the one or more network functions.

The processor (830) may read a computer program stored in the memory (840) to provide a sleep analysis model according to one embodiment of the present disclosure. According to one embodiment of the present disclosure, the processor (830) may use the sleep analysis model to perform a sleep analysis of the user based on the sleep sound information.

In other words, the user's breathing during sleep includes a lot of information for analyzing sleep. In addition to body movements and breathing sounds during sleep, the user's breathing during sleep includes a lot of information about various sleep disorders (e.g., sleep apnea, sleep hypopnea, snoring) etc., which may provide high accuracy when utilizing artificial intelligence (AI).

As shown in FIG. 32 (b), during the sleep stage, the user's breathing pattern, regularity, movement sounds and breathing sounds during sleep may be measured, and the recovery breathing sounds after an apnea event and the unstable breathing sounds during a hypopnea event may be measured.

Furthermore, when the frequency patterns of breathing sounds are analyzed, it is possible to make fundamental predictions about the cause of snoring and sleep apnea.

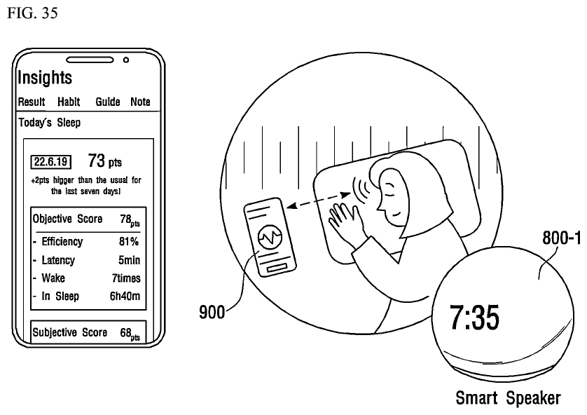

In particular, the breathing sound during sleep is information about the user's breathing during sleep that may be conveniently measured away from a hospital through various smart home appliances (800), such as a smartphone (900) or a smart speaker etc., as shown in FIG. 35.

The processor (830) may perform calculations to train a sleep analysis model. Based on the sleep analysis model, sleep information regarding the user's sleep stages, sleep quality, occurrence of sleep disorders etc. may be inferred. The sleep sound information acquired from the user in real time or periodically is input to the sleep analysis model to output data related to the user's sleep (such as a data related to sleep stages, sleep quality, and occurrence of sleep disorders, etc.).

On the one hand, the smart home appliance (800) according to the present disclosure may further include an alarm unit (850). The alarm unit (850) is a means for providing tactile or sound feedback to the user in the case a sleep disorder such as sleep apnea, occurs during the first and second sleep analysis.

For example, the alarm unit (850) may be implemented as an actuator, vibration module or haptic module to generate a vibration, or a speaker module to generate a sound.

On the one hand, in the present disclosure, the sleep status information may be information about whether the user is sleeping. Specifically, the sleep status information may include at least one of a first sleep status information that the user is before sleeping, a second sleep status information that the user is during sleep, and a third sleep status information that the user is after sleeping.

In other words, if the first sleep state information is inferred related to the user, the processor (830) may determine that the user is in a pre-sleep state (i.e., before bedtime). If the second sleep state information is inferred, the processor (830) may determine that the user is in a sleeping state. And if the third sleep state information is acquired, the user may be determined to be in an after-sleep state (i.e., awake).

Such sleep state information may be acquired based on environment sensing information. The environment sensing information may be sensing information acquired from the space where the user is located in a non-contact manner.

For example, the processor (830) may extract sleep state information based on environment sensing information (such as sound information related to cleaning, sound information related to cooking food, sound information related to watching TV, sleep sound information acquired during sleep, etc.) acquired from the sensor unit (820).

In this case, the sleep sound information acquired during the user's sleep may include sounds generated by the user tossing and turning during sleep, sounds related to muscle movements, or breathing sounds during sleep. In other words, the sleep sound information in the present invention may mean sound information related to breathing patterns during the user's sleep.

Sleep stages may be categorized as non-REM (NREM) sleep, rapid eye movement (REM) sleep, and NREM sleep may be further categorized into a plurality of stages (e.g., two stages of light, deep, and four stages of N1 to N4). Sleep stage settings may be defined based on commonly accepted sleep stages, but they may also be arbitrary in various ways depending on the designer.

Sleep stage analysis may be used to predict not only sleep quality, but also sleep disorders (e.g., sleep apnea) and their underlying causes (e.g., snoring).

The processor (830) may acquire sleep state information based on the sound information acquired form the smart home appliance (800). Specifically, the processor (830) may identify singularities in the sound information where information of a preset pattern is detected.

Here, the information of a preset pattern may relate to a breathing pattern related to sleep. For example, in the wake state, the breathing pattern may be irregular and there may be a lot body movement because all nervous systems are active.

Also, neck muscles are not relaxed, so breathing sounds may be very low. On the other hand, when the user is asleep, the autonomic nervous system stabilizes and the breathing becomes more regular and louder.

In other words, the processor (830) may identify as a singularity a point in the sound information at which a sound information of a preset pattern regarding regular breathing, low breathing sounds, and etc. is detected. Further, the processor (830) may acquire sleep sound information based on the sound information based on the identified singularity.

The processor (830) may identify a singularity related to a time point in the user's sleep in the time series of acquired sound information, and may acquire the sleep sound information based on the corresponding singularity.

In addition, according to one embodiment of the present disclosure, for example, in an embodiment such as FIG. 1 (c), at least one of the electronic devices shown in FIG. 1 (c) may perform at least one of the above-described operations.

### Comparison Between Prior Sleep Analysis Method and Sleep Analysis Method of the Present Invention

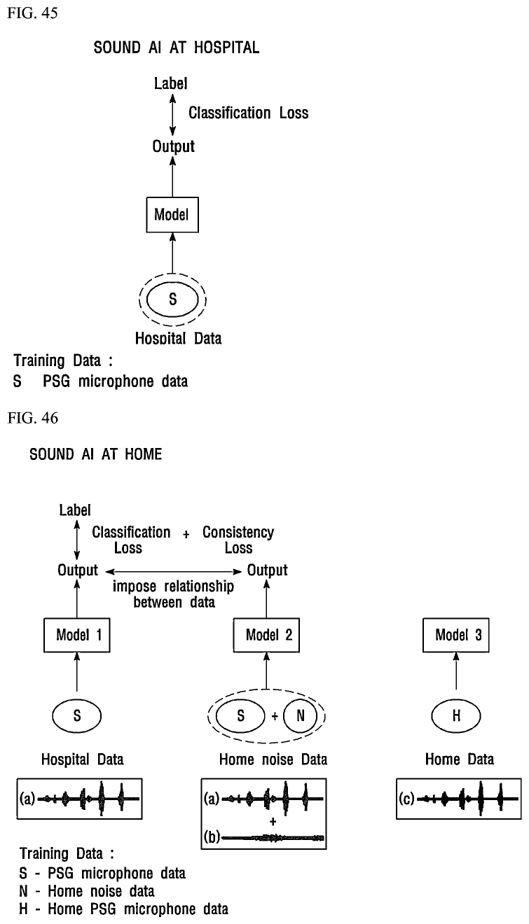

FIG. 45 is a conceptual diagram illustrating a training method using only polysomnography (PSG) microphone data(S) in a hospital environment according to a conventional sleep analysis method, in order to comparing with the sleep analysis method of the present invention.

FIG. 46 is a conceptual diagram illustrating a method for generating an AI sleep analysis model reflecting various sounds in a home environment according to the sleep analysis method of the present invention, in the training method shown in FIG. 45.

Here, waveform (a) is a waveform of polysomnography (PSG) microphone data(S) in a hospital environment, waveform (b) is a waveform of various noise data (N) occurring in a home environment, and waveform (c) is a waveform combined with waveform (a) and waveform (b).

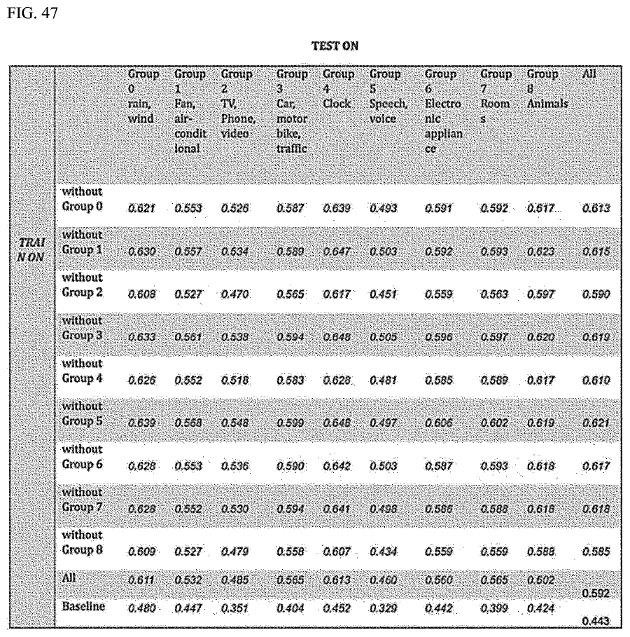

FIG. 47 is a table verifying the performance of the sleep analysis method according to the present invention, which was trained by dividing it into nine groups according to the type of residential noise. FIG. 47 is experimental result data tested for the first to ninth groups (group 0 to group 8).

The types of residential noise are: the first group is rain sound, wind sound, the second group is fan sound, air conditioner sound, the third group is TV sound, telephone sound, video recorder sound, the fourth group is automobile sound, motorcycle sound, other vehicle sound, the fifth group is clock sound, the sixth group is human conversation sound, voice, the seventh group is electronic product sound, the eighth group is inter-room/inter-floor noise, and the ninth group is pet sound.

As shown in FIG. 45, the training method in the case of using only polysomnography (PSG) microphone data(S) in a conventional hospital environment receives polysomnography (PSG) microphone data(S) collected in a hospital as input and outputs it through a first AI sleep analysis model, and a label for sleep analysis and diagnosis reflecting a classification loss is generated and fed back.

On the other hand, the training method in the case of using home polysomnography (PSG) microphone data (H) is as follows.

First, as shown in FIG. 46, the polysomnography microphone data(S) used in the training method (a) only using polysomnography microphone data(S) in a conventional hospital environment is combined with various noise data (N) generated in the home environment.

When this combined data (S+N) is input and output through a second AI sleep analysis model, a consistency loss is generated.

When the classification loss generated by the training method shown in FIG. 20 is added to this consistency loss, a third AI sleep analysis model is generated.

At this time, the first and second AI sleep analysis models impose relevance on each other's output data.

### User's 24-Hour Monitoring Processor and Comparison of Average Results Per Class

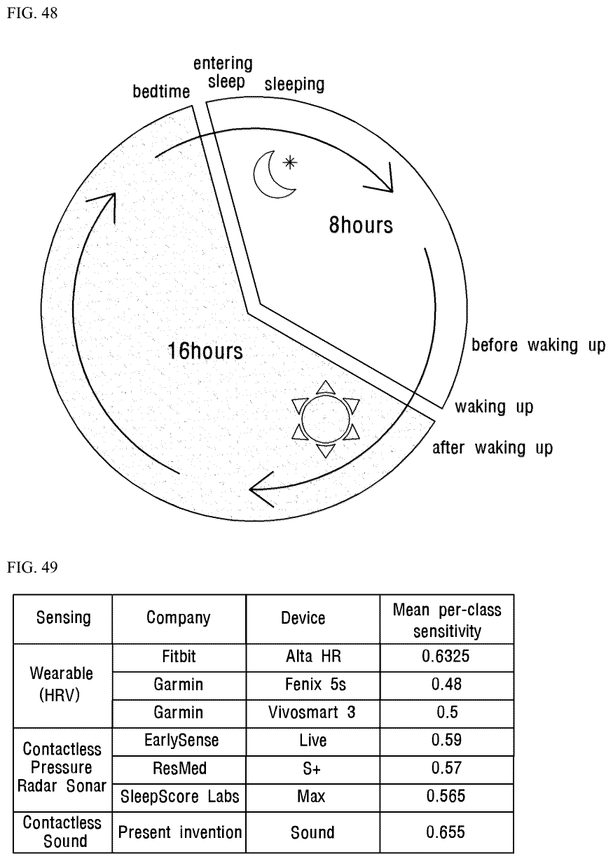

FIG. 48 is a schematic diagram to illustrate a 24-hour monitoring process of a user by an AI-based contactless sleep analysis system and sleep analysis method according to the present invention.

FIG. 49 is a table of mean per class results comparing the smart home appliance and sleep analysis method according to the present invention with the products and devices of existing world-leading sleep technology companies.

In the conventional case where the pattern of user's activity, rest, sleep, etc. were analyzed using only a smartwatch, there is a problem that sleep analysis is interrupted when the smartwatch is taken off during sleep. The present invention enables monitoring of all user activities in real time and seamlessly, even when the smartwatch is taken off during sleep, using a smartphone (900) connected to a smart home appliance (800).

For example, by automatically operating the smartphone (900) when the smartwatch is removed, plugged into a charger, placed on a charging pad, etc., the analysis of the user's activity, rest, sleep, etc. may be continuous. At this time, the smartphone (900) may be operated when it is not adjacent to the smart home appliance (800) and it is time to sleep.

In this way, the continuity of user activity measurement including sleep may be ensured. For example, as shown in FIG. 48, 24-hour data may be acquired from the smartphone (900). And, this data may be processed into various reports and provided to the user.

The user starts the sleep record by touching the screen of the smartphone (900), and receives a sleep analysis result report (bedtime, sleep latency, sleep duration, time to wake up after the alarm, etc.) analyzed in the manner mentioned above. An alarm (an alarm with a sound that gradually increases according to the personal sleep stage, etc.) may be automatically generated according to the sleep stage, and all-day care services such as user profiling (sleep information, preferred content, content recommendation according to age/gender/occupation, etc.), recommendations for personalized sleep/exercise/diet/cosmetics/behavior regulation etc. optimized for personal sleep patterns, may be provided.

The present invention shows weight/blood pressure, sleep apnea, insomnia, or exercise and insomnia as sleep measurement records, which may motivate users to make behavior changes to improve their health. In other words, the present invention may enhance the compliance of user behavior changes very naturally.

For example, if the user is overweight, sleep apnea often occurs, and losing weight may helps improve sleep apnea. Therefore, the present invention may be linked with diet, exercise, and weight tracking in a healthcare application.

In other words, the history of sleep apnea enables behavioral intervention with the real-time sleep apnea detection and accuracy of the present invention.

In addition, since sleep apnea is a cause of high blood pressure, if the respiratory instability unit is regular, blood pressure tracking management is possible using the present invention.

In other words, since weight loss helps lower the body's blood pressure, when weight loss is successful, before and after objective sleep quality may be compared using PSQI.

In addition, exercise (except within 3 hours before bedtime) helps alleviate insomnia, a time facing natural light increases with outdoor activities and the user's mood improves.

In addition, it is possible to receive recommendations for various exercise program from a healthcare application.

In addition, the present invention may show the correlation between stress level and sleep, or premenstrual syndrome and insomnia to the user, and this may make the user re-recognize their health status.

In other words, by indicating the correlation between stress level and sleep quality, interesting elements are added, and depending on the user's stress level and degree of depression, it is possible to fill out a psychiatric related questionnaire provided by the healthcare application.

In addition, if the user complains about insomnia as one of the symptoms of premenstrual syndrome, the sleep efficiency may be marked in the calendar within the menstrual cycle tracking function so that the sleep data may be compared and the user may be checked for health conditions related to the menstrual phenomenon.

One of the important things in the sleep stage analysis is to determine whether the user wakes up during sleep or true awakening occurs. In other words, it is necessary to be able to properly analyze the WAKE stage, the sleep sound signal (sound) is a very useful factor in detecting whether it is in the true WAKE stage.

In the case of brainwave measurement in conventional polysomnography, it was only to check the changed brainwaves in the awakened state of the user, but the sleep sound signal used in the sleep stage analysis of the present invention shows a precursor signal (sound pattern, movement pattern, etc.) from before the user wakes up (before reaching the WAKE stage), and may predict and detect the WAKE stage through this.

According to the AI sleep stage analysis model learned through multiple data, especially the judgment of the WAKE stage based on the sleep sound signal (sound), becomes more precise. In addition, while the user's waking up is accordance with the body's biological rhythm, it may also be affected by external factors (ambient noise, noise, etc.).

The present invention builds an AI sleep stage analysis model by learning the user's sleep environment, i.e., various ambient noises such as routine noses, abnormal or intermittent noises in the surrounding space. Therefore, it may predict and detect the WAKE stage more clearly and reliably.

In fact, as shown in FIG. 49, compared to existing solutions from the world's leading sleep tech companies, the results showed a 43% improvement in Wake accuracy compared to existing wearables, and a 52% improvement compared to existing contactless wearables. The result showed 16% improvement in Wake/Sleep average accuracy compared to existing wearables, and a 20% improvement compared to existing contactless wearables.

In addition, the result showed a 15% improvement in Wake/NREM/REM (3C) average accuracy compared to existing wearables, and a 25% improvement compared to existing contactless wearables.

In addition, the sleep analysis of the present invention using sleep sound information is highly versatile and may be applied to various devices, because anyone can perform sleep analysis as long as there is a device including a microphone, etc.

The method for analyzing sleep, the method for alleviating or preventing sleep disorders, the method for improving sleep disorders and the method for monitoring sleep according to the present invention may be provided by a server providing cloud computing services. More specifically the method of analyzing sleep, the method of alleviating or preventing sleep disorders, the method of improving sleep, and the method of monitoring according to the present invention are a type of internet-based computing. They can be executed by a server that processes information to another computer connected to the internet rather than to the user's computer.

In other words, in the embodiment shown in FIGS. 50 and 51, various sleep sound information acquired from the smart home appliance (800) and the smartphone (900) is transmitted to the AI server (310). The AI server (310) may use the information to perform a sleep analysis and then send the results back to the smart home appliance (800) and the smartphone (900).

According to another embodiment of the present disclosure, various sleep sound information acquired from the smartphone (900) may be converted into spectrograms on the smartphone (900) and transmitted to the AI server (300). In this case, the AI server (310) may use the spectrograms to perform sleep analysis.

According to another embodiment of the present disclosure, various sleep sound information acquired from the smartphone (900) may be converted into spectrograms on the AI server (310), and the AI server (310) may use the spectrograms to perform a sleep analysis.

A cloud computing service may be a service that stores materials on the Internet and may be available to users anytime, anywhere via Internet access without the user installing the required data or programs on their computers. The clouding computer service make it easy to share and deliver data stored on the Internet with simple taps and clicks. Furthermore, the cloud computing service do not only store data on servers on the Internet, but also allow users to perform tasks using the functions of applications provided on the Web without installing any programs. The cloud computing service may be a service that allows multiple people to share documents and work on them at the same time.

In addition, the cloud computing service may be implemented in the form of at least one of infrastructure as a service (IaaS), platform as a service (PaaS), software as a service (SaaS), virtual machine-based cloud server, and container-based cloud server. In other words, the smart home appliance (800) of the present invention may be implemented in the form of at least one of the above-described cloud computing service. The specific description of the cloud computing service described above is by way of example only, and may include any platform for building the cloud computing environment of the present invention.

The sleep analysis methods, sleep disorder mitigation and prevention methods, sleep disorder improvement methods, and monitoring methods according to the present invention may be implemented in the form of program commands that can be performed through various computer means and recorded on a computer-readable medium. The computer-readable recording medium may include program instructions, data files, data structures etc. singly or in combination. The program instructions recorded on the medium may be specifically designed and configured for the present invention or may be known and available to those skilled in the art of computer software. Examples of computer-readable recording media include magnetic media such as hard disk, floppy disk and magnetic tape, optical media such as hard disk, floppy disk and magnetic tape, optical media such as CD-ROM and DVD, magneto-optical media such as floptical disk and hardware devices specifically configured to store and perform program instructions such as ROM, RAM, flash memory etc. Examples of program instructions include a high-level language code that may be executed by a computer using an interpreter etc. as well as a machine language code, which may be adjusted by a complier. The hardware device may be configured to operate as one or more software modules to perform the operation of the present invention, and vice versa.

Alternatively, in an embodiment such as FIG. 1 (c), for example, at least one of the electronics shown in FIG. 1 (c) may perform at least one of the actions described above.

### Generating Environment Adjustment Information

According to one embodiment of the present disclosure, the processor (130) or the processor (830) may generate environment adjustment information based on sleep state information and/or sleep stage information.

The sleep state information is information regarding whether the user is sleeping. The sleep state information may include at least one of first sleep state information indicating that the user is before sleep, second sleep state information indicating that the user is during sleep and third sleep state information indicating that the user is after sleep. The steps of generating the environment adjustment information will be described in detail below using the processor (130) as an example.

According to an embodiment, the processor (130) may generate the first environment adjustment information based on the first sleep state information. Specifically, if the processor (130) acquires the first sleep state information that the user is in a before sleep, the processor (130) may generate the first environment adjustment information based on the first sleep state information.

According to an embodiment, the first environment adjustment information may be information about a light intensity and illumination level that naturally induces sleep. Specifically, the first environment adjustment information may be control information to provide 3000K white light at an illuminance of 30 lux from the based on the time of sleep induction until the time when the second sleep state information is acquired.

According to an embodiment, the time to induce sleep may be determined by the processor (130). Specifically, the processor (130) may determine the time to induce sleep through information exchange with the user terminal (10) of the user. In a specific example, the user may set a time when the user wishes to sleep through the user terminal (10) and transmit to the processor (130). The processor (130) may determine a time to induce sleep based on the time the user wishes to sleep from the user terminal (10). For example, the processor (130) may determine the sleep induction point as 20 minutes before the time the user wishes to sleep based on the time the user wishes to sleep. As a specific example, if the user's set time to sleep is 11:00, the processor (130) may determine 10:40 as the sleep induction time. The specific numerical descriptions of the above time points are examples only, and the present disclosure is not limited thereto.

In addition, according to an embodiment, the processor (130) may acquire the user's sleep intent information based on the environment sensing information and determine the sleep induction time based on the sleep intent information. The sleep intent information may be a quantitative value of the user's intent to sleep. For example, a higher sleep intention of the user may result in a sleep intention information close to 10, and a lower sleep intention may result in a sleep intention information close to 0.

Alternatively, in an embodiment such as FIG. 1 (c), for example, at least one of the electronics shown in FIG. 1 (c) may perform at least one of operations described above.

The foregoing specific numerical description of sleep intent information are by way of example only, and the present invention is not limited thereto.

### Acquiring Sleep Intent Information

The processor (130) or the processor (830) may acquire sleep intent information based on the environment sensing information. Alternatively, in an embodiment such as FIG. 1 (c), for example, at least one of the electronics shown in FIG. 1 (c) may acquire the sleep intent information. The steps of acquiring the sleep intent information will be described in detail below, using processor (130) as an example.

According to one embodiment, the processor (130) may identify a type of sound included in the environment sensing information. Further, the processor (130) may calculate sleep intent information based on the number of identified types of sound. The processor (130) may calculate a lower sleep intent information based on a higher number of sound types, The larger the number of the sound types, the lower the sleep intent information may be calculated by the processor (130), and the smaller the number of the sound types, the higher the sleep intent information may be calculated by the processor (130). In a specific example, if the environment sensing information includes three types of sounds (e.g., a vacuum cleaner sound, a TV sound, and a user voice), the processor (130) may calculate the sleep intent information as a score of 2. Also, for example, if the environment sensing information includes one type of sound (e.g., a washing machine), the processor (130) may calculate the sleep information as a score of 6. The foregoing specific numerical description of the types of sounds included in the environment sensing information and the sleep intent information are examples only, and the present invention is not limited thereto.

In other words, the processor (130) may acquire sleep intent information regarding how much the user intends to sleep based on the number of types of sounds included in the environment sensing information. For example, the more types of sounds are identified, the more sleep intent information may be output that indicates the user's intent to sleep is low (i.e., low-scoring sleep intent information).

In addition, in embodiments, the processor (130) may generate or record an intent score table by pre-matching a different intent score to each of the plurality of sound information. For example, a first sound information related to a washing machine may be matched with an intent score of 2. A second sound information related to a sound of humidifier may be pre-matched with an intent score of 5. And a third sound related to a voice may be matched with an intent score of 1. The processor (130) may pre-match relatively high intent scores for sound information related to the user's sleep (e.g., sounds generated by the user's activity, such as a vacuum cleaner, washing dishes and voice sounds, etc. The processor (130) may generate an intent score table by pre-matching relatively low intent scores for sound information which is not related to the user's sleep (e.g., sounds unrelated to the user's activity, such as vehicle noise, and rain etc.). The specific numerical descriptions of the intent score matched to each of the above sound information are examples only, and the present invention is not limited thereto.

The processor (130) may acquire the sleep intent information based on the environment sensing information and the intent score table. Specifically, in response to a time point at which at least one of the plurality of sounds included in the intent score table is identified in the environment sensing information, the processor (130) may record intent score as 2 matched to the corresponding vacuum cleaner sound in response to the first time point. As each of the various sounds is identified during the process of acquiring the environment sensing information, the processor (130) may record an intent score matched to the identified sound at the corresponding time point.

In embodiments, the processor (130) may acquire sleep intent information based on a sum of intent scores acquired over a predetermined time (e.g., 10 minutes). In a specific example, the higher the intention score acquired during the 10 minutes, the higher the sleep intention information may be acquired. The lower the intention score acquired during the 10 minutes, the lower the sleep intention information may be acquired. The foregoing specific numerical descriptions of predetermined time are examples only, and the present disclosure is not limited thereto.

In other words, the processor (130) may acquire sleep intention information regarding how much the user intends to sleep based on the characteristics of the sounds included in the environment sensing information. For example, the more sounds related to the user's activity are identified, the more sleep intent information may be output indicates the user's intent to sleep is low (i.e., low-scoring sleep intent information).

### Determination of Environment Adjustment Information and Operations of the Smart Home Appliance

According to embodiments of present disclosure, the processor (130) or the processor (830) may determine environment adjustment information based on sleep state information and/or sleep intent information.

Further, various smart home appliance (800) may be operated based on the environment adjustment information according to embodiments of the present disclosure.

Alternatively, in an embodiment such as FIG. 1 (c), for example, at least one of the electronic devices shown in FIG. 1 (c) may perform at least one of the operations described above. Hereinafter, the determination of the environment adjustment information and the operation of the smart home appliance will be described in detail using figures and the like.

### Overall Operations

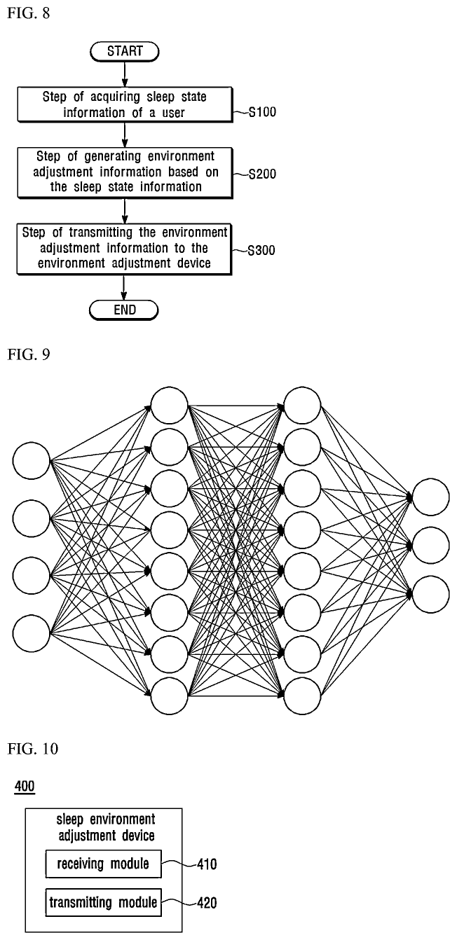

FIG. 8 depicts an exemplary flowchart for providing a method of sleep adjustment environment based on sleep state information, related to one embodiment of present disclosure.

According to one embodiment of the present disclosure, the method may include a step of acquiring sleep state information of a user (S100).

According to one embodiment of the present disclosure, the method may include the step of generating environment adjustment information based on the sleep state information (S200).

According to one embodiment of the present disclosure, the method may include a step (S300) of transmitting the environment adjustment information to the environment adjustment device (30).

The steps depicted in FIG. 8 may be reordered as necessary, and at least one or more steps may be omitted or added. In other words, the steps described above are only one embodiment of the present disclosure, and the scope of the present invention is not limited thereto.

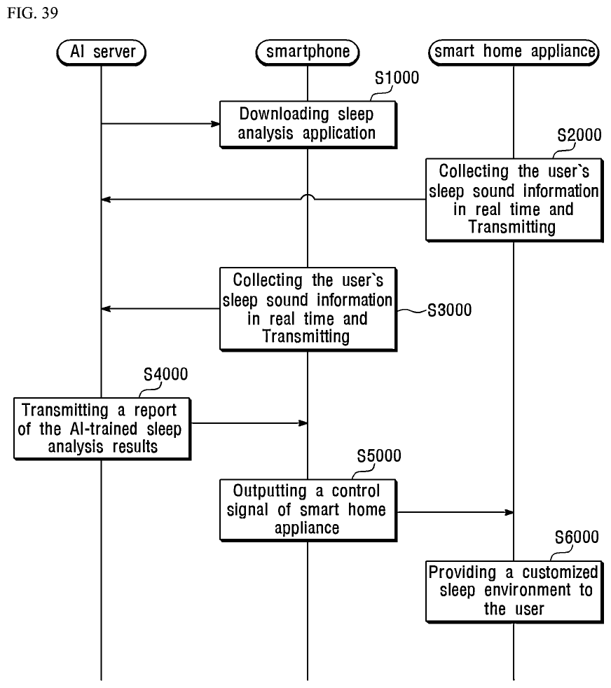

FIG. 39 is a flowchart to illustrate the operation of an AI-based contactless sleep analysis method according to the present invention.

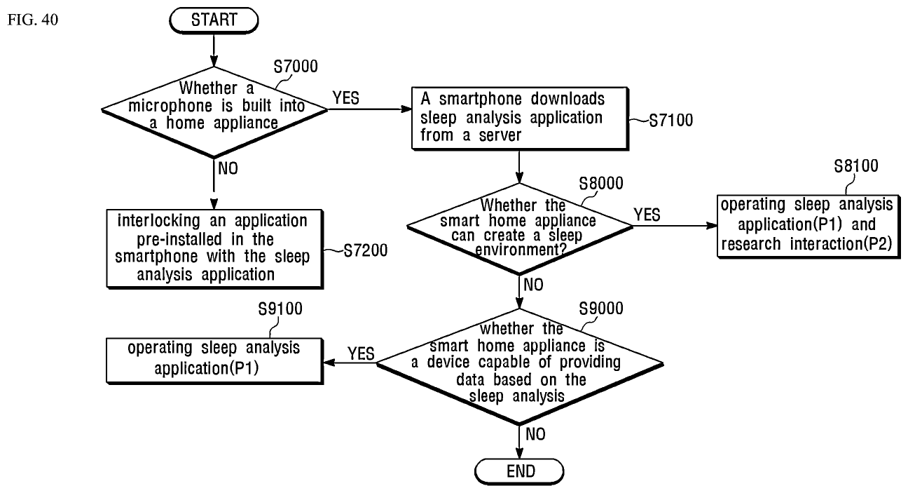

FIG. 40 is a flowchart depicting embodiments of various smart home appliances used in the sleep analysis method according to the present invention.

Referring to FIG. 50, FIG. 51 and FIG. 39, the overall operation of the AI-based contactless sleep analysis according to the present invention is schematically described as follows.

A sleep analysis application may be downloaded to a smartphone (900) (S1000).

At least one smart home appliance (800) may collect the user's sleep sound information in real time and transmit it to the server (310) (S2000).

The smartphone (900) may simultaneously collect the user's sleep sound information in real time and transmit it to the server (310) (S3000).

The server (310) may transmit a report of the AI-trained sleep analysis results to the smartphone (900). (S4000).

The smartphone (900) may output a control signal to control the operations of the at least one smart home appliance (800) (S5000).

At least one smart home appliance (800) may provide a customized sleep environment to the user (S6000).

Next, with reference to FIG. 50, FIG. 51 and FIG. 40, detailed operations of an AI-based contactless sleep analysis method according to the present disclosure will be described as follows.

First, it may be determined whether the smart home appliance (800) has a built-in microphone (S7000).

If it is positive, a sleep analysis application (hereinafter referred to as a sleep track application) according to the present disclosure may be downloaded to the smartphone (900). If it is negative, the sleep track application may be linked to an application already installed on the smartphone (900) (S7200).

Here, the characteristics of the sleep track application are as follows.

As a sleep analysis application that detects user's real-time sleep stages and respiratory instability zones, it has a database that can store weekly and monthly sleep quality indicator and sleep environment, and a dashboard that can derive service insights through usage sessions, sleep statistics, etc. to calculate high-accuracy sleep stage graph (hypnogram), sleep evaluation indicator and respiratory instability indicator for a single night.

The sleep track application also enables seamless monitoring data between wake and sleep in a contactless manner, without the need for a separate wearable device.

Through this, not only allows for increased freedom of movement during sleep, but also allows for accurate timing of wakefulness, which is the foundation of all sleep therapy, making it convenient and accurate to analyze the sleep of different types of users at home, regardless of time and location.

Also, the sleep track application may be used for the following purposes.

It provides a sleep pattern analysis report for each user to intervene in the user's sleep based on real-time sleep tracking and adjust an optimal sleep environment for the user based on sleep analysis result. Furthermore, it may provide alarm, sleep hygiene guide and elevation/wake-up sound content customized to individual sleep stage.

In addition, user profiles such as user-specific sleep information, preferred content, sleep BTI, and reactivity of recommended content according to age group, gender and occupation and content that may form behavior modification and sleep routines such as customized exercise and eating optimized for personal sleep patterns may be recommended.

On the one hand, in the step (S7100), it is determined whether the smart home appliance may adjust a sleep environment (S8000), wherein the sleep environment may include temperature, humidity, light, sound, head and body position, scent etc.

In step (S8000), if positive, the sleep track application may be operated and a study interaction may be adjusted at the same time (S810). In the step (S8000), if negative, it may be determined whether the device is capable of providing customer value, i.e., data, based on sleep analysis via various user interfaces (e.g., PUI, VUI, and/or GUI).

In step (S9000), if positive, the sleep track application may be operated (S9100), and in the step (S9000), if negative, the operation may be terminated since the introduction of the sleep track application is not meaningful.

For example, a smart home appliance reaching step (S8100) may include an air conditioner and/or air purifier to regulate temperature, a humidifier and/or dehumidifier to regulate humidity, blinds and/or curtains to regulate light, a light fixture, a smart speaker to regulate sound, a smart bed to regulate a user's head and body position, a smart diffuser to regulate scent, and a smart device with a healthcare application, etc.

Further, the smart home appliance reaching step (S9100) may include a television, a garment manager, a robotic vacuum cleaner, a washing machine and/or dryer, a refrigerator, a smart device with a healthcare application etc.

In addition, applications that may reach the “sleep management application interaction” besides step (S8100) and step (S9100) may include industries related to fragrance, cosmetics, dietary supplements, traditional sleep industry, sports, hotel, remedial education, fire department and government agency.

In this case, “sleep management application” refers to a type of sleep management application that may analyze sleep without a hardware solution.

In addition, “sleep track application” may refer to a sleep analysis application that delivers a user's sleep report to the user's smartphone (900) in real time via a PUI, VUI, and/or GUI, and operates the smart home appliance (800) according to the report result.

The steps shown in the foregoing figures may be reordered as necessary, and at least one or more steps may be omitted or added. In other words, the foregoing step is only one embodiment of the present disclosure, and the scope of the present invention is not limited thereto.

Hereinafter, the steps of determining the environment adjustment information will be described in detail using the processor (130) as an example and dividing it into sleep state and sleep stage. Furthermore, an example of a smart home appliance (800) that operates according to the environment adjustment information will be described in detail. However, the invention is not limited to the embodiments described below.

### Determination on When to Induce Sleep

According to an embodiment, the processor (130) may determine when to induce sleep based on the sleep intent information. Alternatively, in an embodiment such as FIG. 1 (c), for example, at least one of the electronics shown in FIG. 1 (c) may determine when to induce sleep. Specifically, the processor (130) may identify a time when the sleep intent information exceeds a predetermined threshold score as a time to induce sleep. In other words, when the processor (130) acquires high sleep intent information, it may identify as an appropriate time to induce sleep, i.e., a time to induce sleep.

As described above, the processor (130) may determine when to induce sleep for a user. According to an embodiment, if the processor (130) acquires first sleep state information that the user is pre-sleep, the processor (130) may generate first environment adjustment information (providing white light of 3000K at an illuminance of 30 lux) to adjust the light from the time of sleep induction to the time of acquiring second sleep state information.

### First Environment Adjustment Information and Smart Home Appliance Operations Based on Pre-Bedtime State

According to one embodiment of the present disclosure, if the user's state is a pre-sleep state, the processor (130) may generate first environment adjustment information that causes light to be adjusted from a time when the user is predicted to be ready to sleep (e.g., a sleep induction time) to a time when the user falls asleep (i.e., a time when the second sleep state information is acquired), and may determine to transmit that first environment adjustment information to the environment adjustment device (30).

Alternatively, in an embodiment such as FIG. 1 (c), for example, at least one of electronic devices shown in FIG. 1 (c) may perform at least one of the actions described above.

Accordingly, white light of 3000K may be provided at an illuminance of 30 lux from 20 minutes before the user falls asleep (e.g., at the time of sleep induction) until the moment the user falls asleep. This is an excellent light for melatonin secretion before the user falls asleep, and may improve the user's sleep efficiency by naturally inducing sleep.

In addition, according to an embodiment, the processor (130) may generate first environment adjustment information for controlling the smart home appliance from a time when the user predicted to be preparing to sleep (e.g., a sleep induction time) to a time when the user falls asleep (i.e., a time when the second sleep state information is acquired), if the user's state is a pre-sleep state.

Specifically, the first environment adjustment information may be generated, such as removing the fine dust and harmful gas in advance by a predetermined time (e.g., 20 minutes before sleeping), or controlling the temperature and humidity of the indoor for sleeping. The first environment adjustment information may also include information such as controlling a smart home appliance to cause noise (white noise) at a level that may induce sleep just before sleep, adjusting the blowing intensity of a smart home appliance such as an air purifier or an air conditioner to a preset intensity or less, reducing the intensity of an LED or switching from direct wind to indirect wind. The first environment adjustment information may also include information for controlling the smart home appliance to perform dehumidification/humidification based on the temperature and humidity information in the sleeping space. Further, the first environment adjustment information may include control information for controlling the personalized temperature, humidity, blowing power and noise according to the operation history of the smart home appliance such as an air purifier and air conditioner and the sleep state (sleep quality) acquired.

According to an embodiment of the present disclosure, if the user's state is a pre-sleep state, the smart home appliance may operate according to the first environment adjustment information from a time when the user is predicted to be preparing to sleep (e.g., a sleep induction time) to a time when the user falls asleep (i.e., when the second sleep state information is acquired). The operation of various smart home appliances will be described below using examples.

For example, at a stage when the user is preparing to sleep such as a time when the user is predicted to be preparing to sleep or a time when the user intends to sleep, motion sensors embedded in light fixtures installed in the bedroom, living room, kitchen, bathroom etc. may detect the presence of the user. Furthermore, a healthcare application may initiate sleep measurement of the user.

The TV according to embodiments of the present disclosure may provide user-optimized sleep content or set a screen off time. Here, the user-optimized sleep content may include mindfulness, guided imagery, ASMR, counting backwards, counting sheep etc.

The air conditioner and/or air purifier according to one embodiment of the present disclosure may adjust the room temperature for the user's entering sleeping. It may also switch the type of air provided to indirect air.

The humidifier and/or dehumidifier according to one embodiment of the present disclosure may be activated at a low noise level and/or maintain an appropriate humidity level.

A refrigerator according to one embodiment of the present disclosure may recommend foods that are helpful for sleep (e.g., warm milk, chamomile, etc.) based on an analysis of the user's personal bedtime, or may discourage the user from eating late-night snacks.

The garment manager according to one embodiment of the present disclosure may switch to a silent mode or a bedtime start time may be set to activate immediately upon waking.

Blinds and/or curtains according to one embodiment of the disclosure may be automatically closed, and a sleep light may be switched to a low light. All other light fixtures may be set to turn off.

Further, according to embodiments of the present disclosure, at the time the user falls asleep, the healthcare application may recognize the user's entering the sleep. The TV may be set to turn off the screen while continuing to provide sound-related content of the user-optimized sleep content.

### Second Environment Adjustment Information and Smart Home Appliance Operations Based on Second Sleep State Information

According to one embodiment of the present disclosure, the processor (130) may generate the second environment adjustment information based on the second sleep state information. Further, for example, the processor (130) may identify a time when the user enters sleep, i.e., a sleep entering time through the second sleep state information, and may generate the second environment adjustment information based on the sleep entering time.

For example, the processor (130) may minimize or control smart home appliances to sleep mode from the time of sleep entering, as shown in FIG. 7. As a result, the processor (130) may generate secondary environment adjustment information to optimize temperature and humidity and create a quiet, darkroom-like atmosphere. This secondary environment may help users fall into a deep sleep and improve sleep quality.

In embodiments, the processor (130) may generate external environment adjustment information based on the sleep stage information. In embodiments, the sleep stage information may include information about changes in a user's sleep stage acquired time series through analysis of sleep sound information.

Alternatively, in an embodiment such as FIG. 1 (c), at least one of the electronics shown in FIG. 1 (c) may perform the above operations.

The second environment adjustment information may be control information that minimizes illumination to adjust a lightless darkroom environment. For example, if there is light interference during sleep the probability of fragmented sleep may increase, which may make it difficult to achieve a good night's sleep.

Further, the processor (130) may generate second environment adjustment information based on the second sleep state information to control the smart home appliance to reduce the brightness of the display of the smart home to predetermined brightness, turn off the display, operate at a noise below a preset level, adjust the blowing intensity to a preset intensity, adjust the blowing temperature to a preset range, maintain the humidity at a preset temperature or maintain an indirect wind.

Depending on the sleep stage, when in the second environment adjustment information, there is little fear of waking up in the case of deep sleep. Therefore, control information for operating the smart home appliance by improving air quality in the indoor space or optimizing temperature and humidity etc. may be included.

In other words, if the processor (130) detects that the user enters a sleep (or sleep stage) (upon acquiring the second sleep state information), it may generate second environment adjustment information to prevent light from being provided or control the operations of the smart home appliance. Accordingly, the probability of the user falling into a deep sleep may be increased, thereby improving the quality of sleep.

In addition, as a specific example, if the processor (130) identifies from the user's sleep stage information that the user enters a sleep stage (e.g., light sleep) through the user's sleep stage information, the processor (130) may generate external environment adjustment information to optimize the room temperature and humidity, minimize illumination to adjust a light-free dark environment, or control smart home appliances to remove fine dust/harmful gases, regulate air temperature and humidity, turn on LEDs, adjust operating noise levels, air flow, etc. to promote a good night's sleep. In other words, it may improve the sleep efficiency of users by adjusting an optimal light level for each sleep stage, i.e., the optimal sleep environment.

In addition, the processor (130) may generate environment adjustment information for providing an appropriate level of illumination or adjusting air quality according to a change in the user's sleep stage during sleep. For example, when changing from light sleep to deep sleep, fine red light is provided. Alternatively, when changing from REM sleep to light sleep, more various external environment adjustment information may be generated according to a change in a sleep stage, such as lowering illumination or providing blue light. This automatically takes into account the situation during sleep, considering not only before sleep or immediately after waking up but also the sleep experience as a whole. By doing so, it may have the effect of maximizing the user's sleep quality.

The following describes the operation of various smart home appliances based on the second sleep state information as an example.

A healthcare application according to one embodiment of the present disclosure may analyze a user's breathing in real time and provide a stimulus such as a vibration or alarm in the case of apnea.

A television according to one embodiment of the present disclosure may turn off the screen and turn off the sound.

An air conditioner and/or air purifier according to one embodiment of the present disclosure may maintain an appropriate room temperature and indirect airflow. In addition, from change of temperature, the temperature may be adjusted to detect light sleep.

A humidifier and/or dehumidifier according to one embodiment of the present disclosure may maintain a low noise mode and appropriate humidity.

A door lock according to one embodiment of the present disclosure may verify lock status.

The electrical outlet and/or switch according to one embodiment of the disclosure may be switched to a low power mode.

Among the light fixtures according to one embodiment of the disclosure, a sleeping light may be turned off at a preset time (e.g., 15 to 25 minutes later) from the time when the user's entering sleep is recognized.

Meanwhile, the sleep phase during the sleep mode may be further categorized into a default sleep mode, a sleep personalization mode, and a special care mode by option.

The default sleep mode may provide an environment (air, temperature, humidity, light, scent, etc.) that may adjust a comfortable sleep environment by setting the sleep mode default.

The sleep personalization mode may provide a customized sleep mode based on the user's sleep quality based on accumulated user data.

The special care mode may develop and provide a customized sleep mode optimized for each specific user who has sleep discomfort such as itching, overweight, etc.

Alternatively, in an embodiment such as FIG. 1 (c), for example, at least one of the electronic devices shown in FIG. 1 (c) may perform at least one of the actions described above.

### Third Environment Adjustment Information and Smart Home Appliance Operations Based on Wake-Up Induction Time

According to one embodiment of the present disclosure, the processor (130) may generate the third environment adjustment information based on the wake-up induction time. Alternatively, in an embodiment such as FIG. 1 (c), for example, at least one of the electronics shown in FIG. 1 (c) may generate the third environment adjustment information.

For example, the processor (130) may identify a user's wake-up time from the sleep plan information, generate a wake-up time prediction based on corresponding wake-up time and generate the environment adjustment information accordingly. For example, the processor (130) may generate third environment adjustment information to provide 3000K white light based on the bed position starting at 0 lux and gradually increasing the illumination to reach 250 lux 30 minutes before the predicted wake-up time as shown in FIG. 7. This third environment adjustment information may be used to encourage a natural, brisk wake-up in response to the desired wake-up time.

In addition, the processor (130) may determine to transmit environment adjustment information to the environment adjustment device (30). In other words, the processor (130) may generate external environment adjustment based on the sleep plan information to facilitate the user falling asleep or waking up naturally when sleeping or waking up. In doing so, the user's sleep quality may be improved.

In a further embodiment, the processor (130) may generate recommended sleep plan information based on the sleep stage information. Specifically, the processor (130) may acquire information about changes in the user's sleep stages through the sleep stage information. Based on this information, an estimated time to wake up may be set.

For example, a typical sleep cycle during a day may include light sleep, deep sleep, rapid eye moment and REM sleep stages. The processor (130) determines a wake-up time after the REM time by determining that the time after REM sleep is when the user is most likely to wake up refreshed. By doing so, recommended sleep plan information may be generated. Further, the processor (130) may determine to generate the environment adjustment information based on the recommended sleep plan information and transmit it to the environment adjustment device (30). Thus, the user may naturally wake up according to the sleep plan information recommended by the processor (130). In other words, the processor (130) may recommend a time for the user to wake up based on a change in the user's sleep stage, which may be a time when the user's fatigue level is minimized. Therefore, this may have the advantage of improving the user's sleep efficiency.

As described above, the third environment adjustment information may be characterized as control information for providing 3000K white light at a gradually increasing illuminance from 0 lux to 250 lux from the wake-up induction time to the wake-up time. For example, the third environment adjustment information may be control information related to gradually increasing the illuminance starting 30 minutes before the user's wake-up time (i.e., the wake-up induction time). Here, the wake-up induction time may be characterized in that it is determined based on a wake-up prediction time.

In one embodiment, the wake-up induction time may be characterized as being determined based on a wake-up prediction time. The wake-up prediction time may be information about when the user is expected to wake up. For example, the wake-up prediction time may be 7:00 a.m. for the first user. The foregoing specific description of the wake-up prediction time or number is by way of example only, and the present invention is not limited thereto.

The third environment adjustment information may include information for controlling the smart home appliance to induce the wake-up by increasing or decreasing at least one of room temperature, humidity, airflow intensity, noise and vibration at the wake-up time. Further, the third environment adjustment information may include control information for controlling the smart home appliance to generate white noise. This is to gradually induce the wake-up.

The third environment adjustment information may include control information for keeping the noise of the smart home appliance below a preset level after a weather event.

The third environment adjustment information may also include information for controlling the smart home appliance in conjunction with a wake-up prediction time and a wake-up recommendation time. The wake-up recommendation time may be a time automatically extracted according to the user's sleep pattern, and the wake-up prediction time will be described in detail later.

The following describes the operation of the wake-up mode operation of various smart home appliance based on the wake-up induction time and the wake-up time.

In the pre-wake-up phase, the healthcare application according to one embodiment of the present disclosure may analyze the user's sleep and recognize the user's sleep pattern.

The air conditioner and/or air purifier according to one embodiment of the present disclosure may adjust the environment such as indoor air quality, temperature or humidity for the user's wake-up.

During the wake-up phase, the healthcare application according to one embodiment of the present disclosure may operate smart alarm installed in the application when a user's REM sleep is detected or when a change in the user's body temperature is detected.

The humidifier and/or dehumidifier according to one embodiment of the present disclosure may be switched to a normal operating mode.

A garment manager according to one embodiment of the disclosure may start operating in accordance with a wake-up alarm time that is preset during a bedtime preparation phase.

The blinds and/or curtains according to one embodiment of the disclosure may be opened automatically.

The washing machine according to one embodiment of the present disclosure may initiate a washing operation.

Further, the dryer according to one embodiment of the disclosure may initiate a drying operation.

In the after-wake-up phase, the healthcare application according to one embodiment of the present disclosure may display the analyzed user's sleep report on the user's smartphone (900) and provide user-optimized content such as today's weather, top news etc.

The garment manager according to one embodiment of the present disclosure may complete tasks such as caring for dirt or wrinkles, removing odors, sanitizing, drying, etc. of the garment according to a preset outing time.

According to one embodiment of the present disclosure, the robotic cleaner delivers a report to the user's smartphone (900) and acquires, analyzes and reflects user data as needed during this step. The robotic cleaner according to one embodiment of the disclosure may acquire, analyze and reflect user data after completing washing operation of the washing machine and the drying operation of the dryer before the user goes out.

A water dispenser according to one embodiment of the present disclosure may acquire, analyze and reflect user data after automatically dispensing customized water reflecting user preferences.

A refrigerator according to one embodiment of the present disclosure may display a list of recommended and non-recommended breakfast menu and a list of recommended morning exercise on a display unit based on analyzed user sleep and health data installed on the front display.

Oven/microwave oven according to one embodiment of the present disclosure may automatically preheat a menu when one or more of the breakfast recommendations from the refrigerator is clicked, and then acquire, analyze and reflect the user data.

Further, by obtaining, analyzing and reflecting user data from all smart home appliance (800), best sleeping environment (temperature, humidity, air quality, illumination, etc.) based on personal data may be recommended.

Alternatively, in an embodiment such as FIG. 1 (c), for example, at least one of the electronics shown in FIG. 1 (c) may perform at least one of the above-described operations.

### Determination on Wake-Up Prediction Time

In one embodiment, the wake-up prediction time point may be characterized as being predetermined through an information exchange with the user terminal (10) by a user. In a specific example, a user may set a time at which the user wishes to wake up through the user terminal (10) and transmit it to the processor (130). In other words, the processor (130) may acquire a wake-up prediction time based on the time set by the user of the user terminal (10). For example, if a user sets an alarm time through the user terminal (10), the processor (130) may determine the set alarm time as the wake-up prediction time.

In other embodiments, the wake-up prediction time may be characterized as being determined based on a sleep entering time identified through the second sleep state information. Specifically, the processor (130) may identify the user's sleep entering time from the second sleep state information indicating that the user is sleeping. The processor (130) may determine the time of the wake-up prediction based on sleep entering time identified from the second sleep state information. For example, the processor (130) may determine the wake-up prediction time to be a time after eight hours, which is an appropriate amount of sleep, based on the time of sleep entering time. As a specific example, if the sleep entering time is 11:00 p.m., the processor (130) may determine the wake-up prediction time to be 7:00 a.m. The specific numerical descriptions of each of the foregoing times are by way of example only, and the present disclosure is not limited thereto. In other words, the processor (130) may determine the wake-up time based on when the user falls asleep.

In another embodiment, the wake-up recommendation time may be characterized as being determined based on the user's sleep stage information. For example, a user may wake up most refreshed if they wake up in the REM stage. Over the course of a night's sleep, the user may have a sleep cycle of light sleep, deep sleep, light sleep and REM sleep and may be most refreshed when waking up in the REM sleep stage. Preferably, considering the user's optimal or desired sleep duration, the sleep recommendation time may be determined while minimally satisfying the optimal or desired sleep duration.

Accordingly, the processor (130) may use the sleep stage information regarding the user's sleep stage to determine the wake-up recommendation time for the user. As a specific example, the processor (130) may determine that the weather recommendation time is a time when the user is transitioning from the REM sleep stage to another sleep stage (preferably, a time just prior to transitioning from a REM stage to another sleep stage) through the sleep stage information. In other words, the processor (130) may determine the wake-up recommendation time based on the sleep stage information that the user is most likely to wake up refreshed (i.e., in the REM sleep stage).

As described above, the processor (130) may determine the user's wake-up prediction time based on at least one of user setting, sleep entering time, and sleep stage information. Further, if the processor (130) determines a wake-up prediction time that this is a time when the user wants to wake up, the processor (130) may determine a wake-up induction time based on the wake-up prediction time. For example, the processor (130) may determine the wake-up induction time to be 30 minutes before the time the user wishes to wake up. As a specific example, if the user-set desired time to wake up (i.e., the wake-up prediction time) is 7:00 a.m., the processor (130) may determine 6:30 a.m. as the wake-up induction time. The foregoing specific descriptions of times are by way of example only, and the present disclosure is not limited thereto.

In other words, the processor (130) may determine a wake-up time by identifying a wake-up prediction time when the user is expected to wake up, and may generate third environment adjustment information to provide 3000K white light at a gradually increasing illuminance from 0 lux to 250 lux from the wake-up induction time to the wake-up time (e.g., until the user actually wakes up). The processor (130) may determine to transmit that the third environment adjustment information to the environment adjustment device (30). Accordingly, the environment adjustment device (30) may perform adjustment operations related to light in the space where the user is located based on the third environment adjustment information.

Alternatively, in an embodiment such as FIG. 1 (c), for example, at least one of the electronics shown in FIG. 1 (c) may perform at least one of the actions described above. For example, the environment adjustment device (30) may control the light provision module to provide 3000K white light from 0 lux to 250 lux incremental increase in illumination starting 30 minutes before waking up. The foregoing description of the figures is by way of example only, and the present invention is not limited thereto.

### Fourth Environment Adjustment Information and Smart Home Appliance Operations Based on Third Sleep State Information

According to one embodiment of the present disclosure, the processor (130) may acquire the fourth environment adjustment information based on the third sleep state information. Specifically, the processor (130) may acquire sleep disease information of the user. In one embodiment, the sleep disease information may include sleep phase delay syndrome. The sleep phase delay syndrome may be a symptom of a sleep disorder in which a person is unable to fall asleep at a desired time and an ideal sleep schedule is pushed back. According to an embodiment, blue-light therapy may be one method of treating sleep phase delay syndrome, wherein the user is provided with blue light for about 30 minutes after waking up at a desired wake-up time. If this blue light provision is repeated each morning, it may help to reset the circadian rhythm and prevent the person from falling asleep later in the night than normal.

Accordingly, the processor (130) may generate the fourth environment adjustment information based on the sleep disease information and the third sleep state information. For example, if the sleep disease information indicating that the user has sleep phase delay syndrome and the third sleep state information indicating that the user is after sleep (i.e., wake up) are acquired through the user terminal (10), the processor (130) may generate the fourth environment adjustment information. In this case, the fourth environment adjustment information may be control information to provide blue light with an illuminance of 300 lux, a chromaticity of 221 degrees, a chroma of 100% and a brightness of 56% for a preset time from the wake-up time. In one embodiment, the blue light having an illuminance of 300 lux, a chromaticity of 221 degrees, a chroma of 100% and a brightness of 56% may be blue light for treating sleep phase delay syndrome. In a specific example, if a user with sleep phase delay syndrome wakes up at 7:00 a.m., the processor (130) may identify the wake-up time as 7:00 a.m. based on the third sleep state information and generate the fourth environment adjustment information to provide blue light having an illuminance of 300 lux, a chromaticity of 221 degrees, a chroma of 100% and a brightness of 56% from the wake-up time of 7:00 a.m. to a preset time (e.g., 7:30 a.m.). Accordingly, the user's circadian rhythm may be adjusted to a normal range (e.g., falling asleep around 12:00 p.m. and waking up around 7:00 a.m.). In other words, the generation of the fourth environment adjustment information may improve sleep quality for users with certain sleep disorders.

According to one embodiment of the present disclosure, the processor (130) may determine to transmit the environment adjustment information to the environment adjustment device. Specifically, the processor (130) may generate environment adjustment information about adjusting light level. Further, by determining to transmit the environment adjustment information to the environment adjustment device (30), the processor (130) may control the illumination adjustment operation of the environment adjustment device (30).

According to embodiments, light may be one of the primary factors that may affect sleep quality. For example, illumination, color, exposure etc. of light may have positive and negative effects on sleep quality. Accordingly, the processor (130) may adjust light levels to improve a user's sleep quality. For example, the processor (130) may monitor a situation before falling asleep or after falling asleep and may make light adjustments accordingly to wake the user up effectively. In other words, the processor (130) may identify a sleep state (e.g., sleep stage) and automatically adjust the light to maximize sleep quality.

Alternatively, in an embodiment such as FIG. 1 (c), for example, at least one of the electronics shown in FIG. 1 (c) may perform at least one of the actions described above. The foregoing descriptions of figures and time are by way of example only, and the present invention is not limited thereto.

### Environment Adjustment Information Based on Detected Events

One embodiment of the present disclosure may generate environment adjustment information for controlling an environment adjustment device based on at least one detected event.

Generation of environment adjustment information according to one embodiment of the present disclosure may be performed on the computing device (100) shown in FIG. 1 (a) or the sleep environment adjustment device (400) shown in FIG. 1 (b).

The event may be preset in at least one or more different ways. For example, the event may include at least one of the following events A through H.
A. in room
B. in bed
C. Fall asleep
D. Apnea
E. Deep sleep
F. Wake up during sleep
G. REM near alarm
H. Wake up

Below, each event is described in detail.

The A event above is an event indicating that the user enters a sleeping space, e.g., a bedroom. The A event may be detected by a presence detection sensor.

The presence detection sensor may also be referred to as an human body detection sensor. The human body sensor may include, for example, radar sensor, PIR motion sensor, Wi-Fi sensing sensor, camera sensor, ultrasonic sensor, etc.

The presence sensor may be mounted on the environment adjustment device (30), or it may be mounted separately in the bedroom and connected wired or wirelessly to the environment adjustment device (30). Alternatively, the presence detection sensor may be connected to the network of FIG. 1 (a) or FIG. 1 (b) to transmit sensing signals to the computing device (100), the sleep environment adjustment device (400), the user terminal (10) or the sleep environment adjustment device (30). Alternatively, the presence detection sensor may be connected through short-range communication with the user terminal (10) to transmit the sensing signal to the user terminal (10).

Alternatively, when the present invention is implemented in the embodiment of FIG. 1 (c), the presence detection sensor may be present in at least one of the electronic devices shown in FIG. 1 (c).

When the A event occurs, in other words, when the A event is detected by the presence detection server, A environment adjustment information may be generated for automatically turning on the environment adjustment device (30). Here, in addition to automatically turning on the environment adjustment device (30), the A environment adjustment information may also include control information to change the environment adjustment device (30) to a specific operation mode.

The B event above is an event indicating that the user is lying in bed. The B event may be detected through a piezoelectric sensor. The piezoelectric sensor may be mounted on the bed in which the user sleeps. However, it is not limited thereto, the piezoelectric sensor may also be mounted on a couch, a massage chair etc. on which the user sleeps.

The piezoelectric sensor may be connected wired or wirelessly to the environment adjustment device (30). Alternatively, the piezoelectric sensor may be connected to the network of FIG. 1 (a) or FIG. 1 (b) to transmit sensing signals to the computing device (100), the sleep environment adjustment device (400), the user terminal (10) or the sleep environment adjustment device (30). Alternatively, the piezoelectric sensor may be connected through short-range communication with the user terminal (10) to transmit the sensing signal to the user terminal (10).

When the B event occurs, in other words, when the B event is detected by the piezoelectric sensor, B environment adjustment information for operating the environment adjustment device (30) into a sleep mode may be generated. The B environment adjustment information may include control information of the environment adjustment device (30) for falling asleep of the user. The control information may include information for reducing noise, light etc. generated by the environment adjustment device (30). For example, if the environment adjustment device (30) is an air conditioner, it may include information such as changing the air volume to a certain intensity or lowering flow rate to a certain intensity or lower, switching from direct wind to indirect wind or no wind, or lowering the brightness of the display part to a certain brightness or lower. The information may also include turning off a light in the bedroom or reducing the brightness of the light to a predetermined level. It may also include information about closing curtains or blinds installed in the bedroom to remove external sleep disturbances. It may also include information for turning on a sound device installed in the bedroom to play a particular sound, or, conversely, turning off the sound device. It may also include information for changing the motion of a motion bed installed in the bedroom to a particular motion that is favorable for reading or watching media before falling asleep. It may also include information to operate a scent generator installed in the bedroom to produce a scent that is conducive to wake up.

The C event above is an event indicating that the user falls into (or enters) a sleep. The C event may be identified by the computing device (100) or the sleep environment adjustment device (400) receiving the environment sensing information sensed by the user terminal (10), as described above. As shown in FIG. 7, a falling (or entering) asleep may be identified from the environment sensing information sensed by user terminal (10).

When the C event occurs, in other words, when the C event is detected, a C environment adjustment information may be generated for operating the environment adjustment device (30) into sleep mode. The C environment adjustment information may include control information for adjusting an optimal bedroom sleep environment. Here, the optimal bedroom sleep environment may be optimal environment information acquired based on pair data (temperature and/or humidity & sleep quality) acquired over a predetermined period of time (e.g., a week, or a month). For example, based on quantitative data indicating the quality of sleep of a user who has been sleeping of the past week and temperature and humidity data of a bedroom during the time that the quantitative data was acquired, the temperature and humidity of the bedroom where the user sleep best may be determined as the optimal bedroom sleep environment. The control information may be to set the temperature and humidity of the bedroom of the environment adjustment device (30) to the optimal temperature and humidity. It may also include information for turning off a light installed in the bedroom. It may also include information for turning off a sound device installed in the bedroom. It may also include information to change the motion of a motion bed installed in the bedroom to a particular motion that is favorable for sleep. Further, the information may include causing a scent generator installed in the bedroom to emit a scent that is conducive to sleep or turning off the scent generator.

The D event above is an event indicating that sleep apnea or hypopnea occurs while the user is sleeping. The D event may be identified by the computing device (100) or the sleep environment adjustment device (400) receiving the environment sensing information sensed by the user terminal (10), as described above. As shown in FIG. 4, the environment sensing information sensed by the user terminal (10) may be used to determine when sleep apnea or hypopnea occurs.

When the D event occurs, in other words, when the D event is detected, a D environment adjustment information may be generated for operating the environment adjustment device (30) into a sleep mode. The D environment adjustment information may include control information for alleviating sleep apnea or hypopnea, or for rapidly converting stopped or weak breathing to normal breathing. For example, the control information may include information to increase a set humidity or temperature, or switch from direct or indirect airflow to no airflow, if the environment adjustment device (30) is an air conditioner, to protect the airway and throat of a user experiencing sleep apnea. Alternatively, if the environment adjustment device (30) includes a humidification function, it may include information to activate corresponding humidification function. Or, if the environment adjustment device (30) includes a vibration function, it may include information to activate the vibration function. It may also include information to cause a light installed in the bedroom to be illuminated at a particular brightness and color temperature. It may also include information to change the motion of a motion bed installed in the bedroom to a particular motion to assist the user in breathing. It may also include information to cause a scent generator installed in the bedroom to produce a scent that may relieve sleep apnea.

The E event above is an event indicating that the user falls a deep sleep. The E event may be identified by the computing device (100) or the sleep environment adjustment device (400) receiving the environment sensing information sensed by the user terminal (10), as described above. As shown in FIG. 3, the environment sensing information sensed by the user terminal (10) may be used to identify when a deep sleep enters.

When the E event occurs, in other words, when the E event is detected, E environment adjustment information may be generated for operating the environment adjustment device (30) into sleep mode. The E environment adjustment information may include control information for changing the temperature or humidity to a temperature or humidity optimized for the deep sleep phase. For example, the control information may include information for changing the current bedroom temperature or humidity to the optimized temperature or humidity when the environment adjustment device (30) is an air conditioner. Here, the optimized temperature or humidity may be determined to be a particular temperature or humidity at which the user has the longest duration of deep sleep using quantitative sleep reports acquired over a predetermined period of time. It may also information turning off or reducing to a minimum brightness a light installed in the bedroom. It may also include information about turning off a sound device installed in the bedroom. It may also include information changing a motion of a motion bed installed in the bedroom to a particular motion that is favorable for deep sleep. It may also include information for causing a scent generator installed in the bedroom to produce a scent that is conducive to deep sleep.

The F event above refers to an event in which the user wakes up during sleep. The F event may be identified by the computing device (100) or the sleep environment adjustment device (400) receiving the environment sensing information sensed by the user terminal (10), as described above. As shown in FIG. 3, a time of wake-up from sleep may be identified from the environment sensing information sensed by the user terminal (10).

When the F event occurs, in other words, when the F event is detected, F environment adjustment information may be generated for operating the environment adjustment device (30) into sleep mode. The F environment adjustment information may include control information to assist the user in falling back to sleep. For example, the control information may include information to change the set temperature and humidity of the air conditioner, to a preferred temperature or humidity that was previously set by the user when falling asleep. Alternatively, the control information may include information to change the set information or humidity of the environment adjustment device (30) to a particular temperature or humidity that was set for the shortest period of time when the user falls asleep in the past using a quantitative sleep report. It may also include information to cause a light installed in the bedroom to be illuminated at a particular brightness and color temperature conducive to sleep. It may also include information for turning on or off a sound device installed in the bedroom. It may also include information changing the motion of motion bed installed in the bedroom to a particular motion that is conducive to falling asleep again of the user. It may also include information to cause a scent generator installed in the bedroom to generate a scent that is conducive to falling asleep again.

The G event above is an event indicating that REM sleep occurs near the preset alarm time. The G event may be identified by the computing device (100) or the sleep environment adjustment device (400) receiving the environment sensing information sensed by the user terminal (10), as described above. As shown in FIG. 3, a time of REM sleep may be identified from the environment sensing information sensed by the user terminal (10).

When the G event occurs, in other words, when the G event is detected, G environment adjustment information may be generated for operating the environment adjustment device (30) into sleep mode. The G environment adjustment information may include control information to help the user wake up. For example, if the environment adjustment device (30) is an air conditioner, the control information may include information for changing a set temperature or humidity of the air conditioner to a particular temperature or humidity that allows the user to wake up naturally or most refreshed. Alternatively, the control information may include information for changing the set temperature or humidity that is most preferred by the user using past quantitative sleep report. It may also include information to cause a light installed in the bedroom to be illuminated at a particular brightness and color temperature specific to the wake-up. It may also include information for opening curtains or blinds installed in the bedroom. It may also include information to turn on a sound device installed in the bedroom to play a particular sound source. It may also include information to change the motion of a motion bed installed in the bedroom to a particular motion that is beneficial to wake-up. It may also include information to cause a scent generator installed in the bedroom to generate a scent that may be beneficial for waking up.

The H event above is an event indicating a time when the user wakes up. The H event may be identified by the computing device (100) or the sleep environment adjustment device (400) receiving the environment sensing information sensed by the user terminal (10), as described above. As shown in FIG. 5, after the singularity (201) is identified in the environment sensing information sensed by the user terminal (10), a wake-up time may be identified by identifying whether a predetermined pattern is continuously detected.

When the H event occurs, in other words, when the H event is detected, H environment adjustment information for operating the environment adjustment device (30) into a sleep mode may be generated. The H environment adjustment information may include control information to set the temperature of the bedroom in which the user sleeps to an optimal temperature after wake-up. If the environment adjustment device (30) is an air conditioner, the control information may include information to change the set temperature or humidity of the air conditioner to a temperature or humidity preferred by the user based past history at the user's wake-up time. Alternatively, the control information may include suggestion information that changes the set temperature or humidity of the air conditioner after the user wakes up to a recommended temperature or humidity through the user terminal. It may also include information to cause a light installed in the bedroom to be illuminated at a particular brightness and color temperature. It may also include information to turn on a sound device installed in the bedroom to display certain media or play certain sounds. It may also include information that causes a window installed in the bedroom to be opened for ventilation. It may also include information to change the motion of the motion bed installed in the bedroom to a particular motion to help the user wake up. It may also include information to cause a scent generator installed in the bedroom to generate a scent that may help the user move after wake-up.

In addition, in case an embodiment of the present disclosure is an embodiment such as FIG. 1 (c), for example, at least one of the electronic devices shown in FIG. 1 (c) may perform at least one of the above-described actions.

### Sleep Plan Information

In one embodiment, the processor (130) may receive sleep plan information from the user terminal (10). The sleep plan information is information that the user generates through the user terminal (10). For example, the sleep plan information may include information regarding a bedtime and a wake-up time. The processor (130) may generate external environment adjustment information based on the sleep plan information. As a specific example, the processor (130) may identify the user's bedtime form the sleep plan information and generate the external environment adjustment information based on the bedtime. Alternatively, in an embodiment such as FIG. 1 (c), for example, at least one of the electronics shown in FIG. 1 (c) may perform at least one of the actions described above.

For example, the processor (130) may generate first environment adjustment information, as shown in FIG. 7, to provide 3000K white light at an illuminance of 30 lux based on the bed position, 20 minutes before bedtime, i.e., to adjust an illuminance that naturally induces the user to fall asleep regarding bedtime. The above-described figures and times are exemplary, and the present invention is not limited thereto.

### Example 1 of Method for Preventing and Alleviating Sleep Disorders

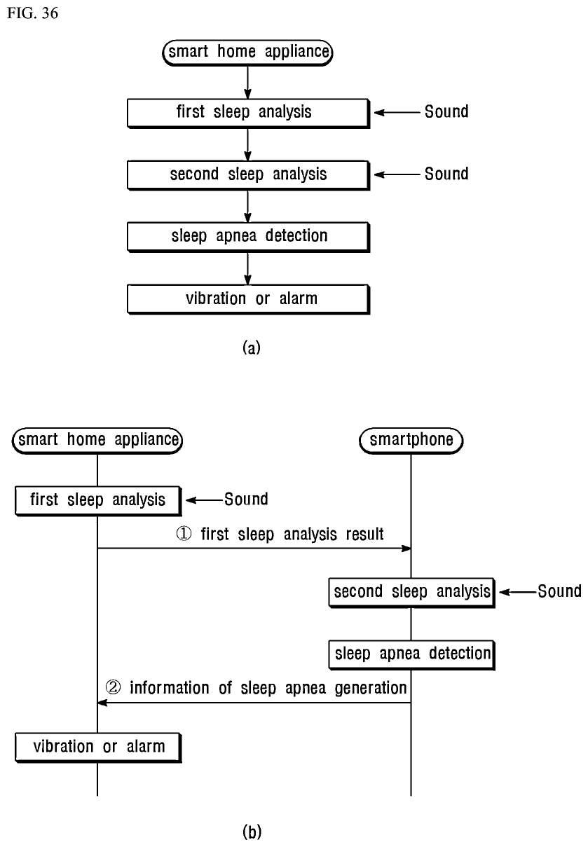

FIG. 36 (a) is a flow diagram illustrating a method for preventing and alleviating sleep disorders using an AI-based contactless sleep analysis system according to one embodiment of the present disclosure.

The present invention may analyze a user's sleep analysis in real time and identify a point where a sleep disorder (sleep apnea, hyperventilation, hypopnea) occurs. By providing the user with a stimulus (tactile stimulus, auditory stimulus, olfactory stimulus, etc.) at the moment the sleep disorder occurs, the sleep disorder may temporarily be relieved. In other words, the present invention may interrupt the user's sleep disorder and reduce the frequency of the sleep disorder based on accurate event detection related to the sleep disorder.

Referring to FIG. 36 (a), a method for preventing and alleviating sleep disorders using smart home appliance (800) according to the present invention collects sleep sound information of a user and performs a first sleep analysis and second sleep analysis based on the information.

The first sleep analysis is a sleep analysis based on the user's sleep sound information, and the second sleep analysis corresponds to an analysis based on the first sleep analysis result and the sleep sound information, and the specific analysis method is the same described above.

As a result of the first sleep analysis and the second sleep analysis, the smart home appliance (800) may generate at least one of tactile feedback and auditory feedback at a time when it is determined that the user is experiencing sleep apnea. The smart home appliance (800) may further include an alarm unit (850) for the feedback, which may be implemented as an actuator, vibration module or haptic module to generate a vibration or a speaker module to generate sound.

Vibration transmitted to a body part (e.g., the entire body in the case of smart mat) in contact with the smart home appliance (800), or acoustic sounds ringing in ears (e.g., a smart speaker, smartphone, smart TV, etc.) may stimulate a user's brain. As a result, sleep apnea is relieved relatively quickly. If this process continues throughout the user's sleep, the frequency of sleep apnea the frequency of sleep apnea decreases significantly.

In this case, not only single apnea events may be detected, but also clusters of apnea events may be predicted in advance. For this purpose, the sleep analysis learning model described above may perform training to predict clusters of sleep apnea events.

In other words, the input information based on the user's sleep sound information is input to the input layer through the preprocessing process and the Mel spectrogram conversion process as described above. After learning this, the sleep analysis learning model may be able to predict a cluster of consecutive sleep apneal events.

If the cluster of consecutive sleep apnea events is predicted in advance, it is possible to prevent sleep apnea, alleviate sleep apnea or improve sleep apnea by vibrating the smartphone (900) once or several times at not only at the moment the sleep but also at a pre-predicted time.

In other words, the present invention is able to analyze sleep stages and alleviate or improve sleep apnea based on sleep sound information signals.

The pattern of tactile and auditory feedback to the user may be intended to reduce the frequency of sleep apnea while maintaining a good night's sleep. These patterns may be adjusted in real time based on analysis of the user's sleep stages.

Further, the patterns may be inferred by a deep learning model trained based on big data about the user's sleep stage analysis results and big data about the frequency of sleep apnea.

In the above, sleep disorders such as sleep apnea and hyperventilation were mentioned, but in order to improve sleep quality, a stimulus may be delivered to the user through the smart home appliance (800) when it is determined that the user is in the REM sleep stage.

REM sleep is a sleep stage in which brain waves are rapid and autonomic nervous activity, such as heart rate and breathing, is irregular, accompanied by mild involuntary muscle twitches and rapid eye movements. It is common to have three to four occurrences approximately 80 to 120 minutes apart, but in severe cases, it may lead to REM sleep disorder and affect sleep quality.

Therefore, in addition to sleep disorders such as sleep apnea, hyperventilation and snoring, the smart home appliance (800) may also stimulate the user at the point of REM sleep. In other words, the smart home appliance (800) may generate at least one of tactile feedback and auditory feedback at a time when, as a result of the first sleep analysis and the second sleep analysis, the smart home appliance (800) determines that the user enters the REM sleep stage.

Alternatively, in an embodiment such as FIG. 1 (c), for example, at least one of the electronics shown in FIG. 1 (c), may perform at least one of the actions described above.

### Example 2 of Method for Preventing and Alleviating Sleep Disorders

FIG. 36 (b) is a flow diagram illustrating a method of preventing and alleviating sleep disorders using an AI-based contactless sleep analysis system, according to another embodiment of the present disclosure.

The embodiment shown in FIG. 36 (b) assumes a situation where sleep analysis is performed on a smart home appliance (800) and a smartphone (900).

The sleep analysis result may include sleep state information, sleep stage information, sleep disorder occurrence information, time information etc. The smartphone (900) performs sleep analysis based on sleep sound information acquired through the built-in microphone module. The following describes how the smartphone (900) uses the sleep sound information to derive a final sleep analysis result.

First, the smartphone (900) may derive a final sleep analysis result using weights. Specifically, the smartphone (900) may derive a first sleep analysis result and a second sleep analysis result by applying equal weights using the sleep sound information.

In other embodiments, the smartphone (900) may derive a final sleep analysis result by determining the user enters a sleep stage only if the sleep stage in the first step analysis result and the second sleep analysis result is completely consistent. In other embodiments, the smartphone (900) may first perform a second sleep analysis using the sleep sound information (sound) using the AI sleep analysis model described below, and then further extract the AI confidence of the sleep stage for each time period.

If the extracted confidence is below the preset value, the sleep stage for that time period will adopt the sleep stage result derived by the first sleep analysis.

In other words, a more reliable sleep analysis result may be derived by adopting the first sleep analysis result based on the second sleep analysis result.

In another embodiment, the smartphone (900) first acquires statistics of a part that is inconsistent with the actual analysis result in the AI sleep analysis model described below. The statistics may be input by the user, but may also be self-acquired by multiple user data. The smartphone (900) may additionally adopt the first sleep analysis result in a prat that is inconsistent with the actual analysis result in the acquired statistics, based on the second sleep analysis result (analysis based on sound).

The learning method of the AI sleep analysis model will be described in more detail below, but in brief, by inputting two types of information (first sleep analysis result and sleep sound information) into the deep learning input layer, an AI sleep analysis model that performs sleep analysis based on two factors may be generated.

This is only an embodiment, and the final sleep analysis result may be derived in various ways.

If the second sleep analysis by the smartphone (900) determines that sleep apnea occurs, the sensor part may immediately transmit the sleep apnea occurrence information to the processor embedded in the smartphone (900). The sleep apnea occurrence information corresponds to a trigger signal for the smart home appliance (800) to generate at least one of tactile feedback and auditory feedback. When receiving the sleep apnea event information, the smart home appliance (800) may stimulate the user through vibration, sound, acoustics etc. The stimulation may quickly relieve the user's sleep apnea, and may prevent or alleviate the user's sleep apnea through continuous monitoring and stimulation.

On the one hand, unlike the embodiments shown in FIG. 34 to FIG. 36, the first sleep analysis may be omitted and the sleep analysis may be performed only on the smartphone (900). In other words, based on the user's sleep sound information, the user's sleep analysis is performed in the above-mentioned manner. If sleep apnea is detected as a result of the sleep analysis, the sleep apnea occurrence information may be immediately transmitted to a smart home appliance (800) (e.g., a smart mat, a smart speaker, etc.) linked to the smartphone (900), thereby causing the smart home appliance (800) to generate a vibration or an alarm (sound, acoustic).

On the one hand, there is a correlation between air quality and sleep.

According to one study, it is known that if the mother is exposed to bad air during the first to eighth week of pregnancy, the baby's sleep efficiency decreases, and when the mother is exposed to bad air during the 31st to 35th week of pregnancy, the baby's sleep time decreases. There is a study research result that the quality of sleep during the growing period is closely related to the ability to acquire knowledge and growth.

In addition, studies shows that exposure to PM 10 in the summer increases breathing irregularities during sleep, which is related to increased cardiovascular disease and mortality in human.

On the one hand, the relationship between temperature/humidity and sleep is as follows.

A study comparing sleep at 880 ppm of carbon dioxide to 17000 ppm of carbon dioxide shows that the air at 800 ppm felt more stuffy and hotter. A study shows the sleeping in a chamber with a temperature of 28 degrees Celsius reduces sleep efficiency and performance of the next day compared to sleeping in a chamber with a temperature of 24 degrees Celsius.

In addition, studies show that sleeping in an environment with 80% of relative humidity and a temperature of 32 degrees Celsius increases the frequency of awakening during sleep and decreases the percentage of deep sleep when compared to sleeping in an environment with 50% of relative humidity and a temperature of 26 degrees Celsius.

The stimulus for preventing or alleviating such sleep disorders in users may be generated by an environment adjustment device other than the smartphone (9000 or smart speaker.

Here, other environment adjustment device may be lighting, air purifier, humidifier, speaker (audio), clothing manager, TV, watch, PC, motion bed, mattress, smart pillow, blind, curtain, robot, vacuum cleaner, washer, dryer, water purifier, refrigerator, oven/range etc. The user's sleep disruption information may be transmitted to the various environment adjustment device mentioned above, and the environment adjustment device may generate a stimulus source to stimulate the user.

For example, the sleep disorder occurrence information may stimulate the user to stop or alleviate the sleep disorder by controlling the lighting (electric light) to increase the illumination, generating the sound of the air purifier, turning on the TV, operating the clock alarm, turning on the PC, controlling the motion bed to change the bed angle, controlling the smart pillow or smart mattress to make tactile changes or movements and operating various home appliances to generate sounds, etc.

In addition, in an embodiment such as FIG. 1 (c), for example, at least one of the electronics shown in FIG. 1 (c) may perform at least one of the actions described above.

### Traffic Response Method and Single-Person/Multiple-Person Sleep Analysis

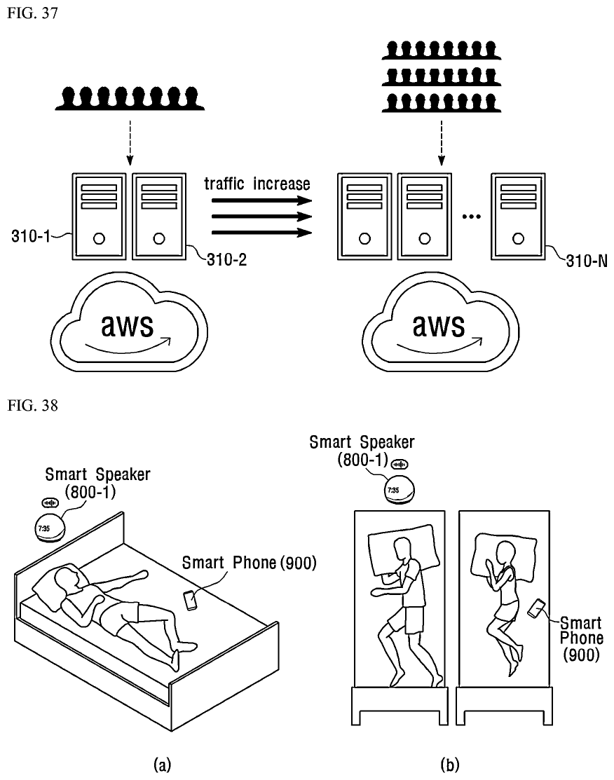

FIG. 37 is a diagram illustrating a traffic response method when a sleep analysis method according to the present invention is performed in the cloud.

FIG. 38 is a conceptual diagram illustrating a single-person sleep analysis and a multi-person sleep analysis in a sleep analysis method according to the present invention. For ease of understanding, the smart home appliance (800) will be described herein as a smart speaker. However, this is for purposes of illustration only, and the smart home appliance of the present invention is not limited to smart speaker.

The sleep analysis method according to the present invention may be provided to a user through the Amazon Web Services (AWS) cloud. Since the sleep analysis method according to the present invention is mainly performed from evening to early morning hours, traffic may be generated during those hours.

Therefore, a sleep analysis method according to the present disclosure may further comprise the steps of analyzing a time period in which a large amount of traffic occurs, predicting an event entering that time period, and automatically adjusting (adding, relocating etc.) the AI server (310) at the time of that event. In this way, the present invention is able to flexibly handle traffic that is likely to occur at a particular time.

First, in the case of a single person sleeping as shown in FIG. 38 (a), in the single person sleep analysis, both the smart speaker and the smartphone (900) are located within the same sleep space, i.e., the smart speaker may obtain sleep sound information, sleep environment information, etc. of the single person. The smartphone (900) may acquire sleep sound information, sleep environment information (illumination etc.) and the like of a single user. In such a single-user sleep environment, the sleep analysis method described above may be applied as it is.

However, in the case of multi-person sleep shown in FIG. 38 (b), the sleep sound information acquired by the smart speaker or smartphone (900) may include may include sleep information of multiple users such as user 1 and user 2.

Therefore, sleep analysis may undergo a more precise process when multiple users are sleeping in the same sleeping space. Since the multi-person sleep analysis method has been previously described with reference to FIGS. 39 to 46, a redundant description will be omitted.

### Sleep Environment Adjustment Device

Hereinafter, the sleep environment adjustment device shown in FIG. 1 (b) will be described in more detail. As shown in FIG. 1 (b), the sleep environment adjustment device (400), the user terminal (10) and the external server (20) may send and receive data through a network to each other for systems according to embodiments of the present disclosure.

The network according to the embodiments of the present disclosure has been described in detail above, and these redundant descriptions will be omitted.

According to the present embodiment, the user terminal (10) is a terminal that may receive information related to the user's sleep through information exchange with the sleep environment adjustment device (400), and may refer to a terminal possessed by the user. The general configuration and function of the user terminal (10) may be described above. The user terminal (10) may acquire sound information related to a space in which the user is located. For example, the sound information may refer to sound information acquired in the space where the user is located. The sound information may be acquired related to the user's activity or sleep using a contactless method.

For example, the sound information may be acquired in the space while the user is sleeping. According to an embodiment, the sound information acquired through the user terminal (10) may be the information that is a basis for acquiring the user's sleep state information in the present invention. For example, in a specific example, the sleep state information related to whether the user is before sleep, during sleep, or after sleep is acquired through the sound information acquired related to movement or breathing of the user. Further, for example, information about changes in sleep stage of the user during a sleep period may be acquired from the sound information.

The sleep environment adjustment device (400) of the present invention may receive medical examination information or sleep examination information etc. from the external server (20), and build a training data set based on such information. A description of the external server (20) has been described in detail above, and will not be described herein.

According to one embodiment, the sound information utilized by the sleep environment adjustment device (400) to analyze sleep conditions may be acquired in a non-invasive manner during a user's activity in the space or during sleep. As a specific example, the sound information may include sounds generated by a user tossing and turning during sleep, sounds related to muscle movements, or sounds related to a user's breathing during sleep. According to an embodiment, the environment sensing information may include sleep sound information, wherein the sleep sound information may refer to sounds related to movement patterns and breathing patterns that occur during the user's sleep.

In embodiments, the sound information may be acquired through at least one of the user terminal (10) and the sound collection unit (414) carried by the user. For example, the environment sensing related to a user's activity in a space may be acquired through a microphone module equipped on the user terminal (10) and the sound collection sensor (414).

The configuration of the microphone module equipped in the user terminal (10) or the sound collection sensor (414) is the same as described above.

The sound information that is subject to analysis in the present invention, which relates to the user's breathing and movements acquired during sleep, is information about very small sounds (i.e., sounds that are difficult to distinguish) and is acquired along with other sounds in the sleep environment. Therefore, it may be very difficult to detect and analyze if it is acquired through a microphone module such as the one described above with a low signal-to-noise ratio.

According to one embodiment of the present disclosure, the sleep environment adjustment device (400) may acquire sleep state information based on sound information acquired through a microphone module configured with MEMS. Specifically, the sleep environment adjustment device (400) may convert and/or adjust the unclearly acquired sound information, including a lot of noise, into analyzable data. The sleep environment adjustment device (400) may utilize the transformed and/or adjusted data to perform training for the artificial neural network. When pre-training of the artificial neural network is complete, the trained neural network (e.g., a sound analysis model) may acquire sleep state information of the user based on the acquired (e.g., transformed and/or adjusted) data (e.g., spectrogram) in response to the sound information. In embodiments, the sleep state information may include information regarding sleep stage information regarding changes in the user's sleep stage during sleep as well as whether the user is sleeping. As a specific example, the sleep state information may include sleep stage information that indicates that indicates that at a first time point, the user was in REM sleep and at a second time point, which is different from the first time point, the user was in light sleep. In this case, through the sleep state information, the information may be acquired that the user was in a relatively deep sleep at the first time point and was in a shallower sleep at the second time point.

In other words, when the sleep environment adjustment device (400) acquires sleep sound information having a low signal-to-noise ratio through a user terminal (e.g., an artificial intelligence speaker, a bedroom IoT device, a cell phone, etc.) or sound collection unit (414) that is commonly used to collect sound, the sleep sound information may be processed into data suitable for analysis, and the processed data may be used to provide information about whether the user is before, during, or after sleep, and sleep state information regarding changes in sleep stages.

In embodiments, the sleep environment adjustment device (400) may be a terminal or a server, and may include any type of device. The sleep environment adjustment device (400) may be a digital device such as a computing-capable digital device having a processor and memory such as laptop computer, notebook computer, desktop computer, web pad, or mobile phone. The sleep environment adjustment device (400) may be a web server that processes the service. The foregoing types of servers are only examples and the present invention is not limited thereto.

According to one embodiment of the present disclosure, the sleep environment adjustment device (400) may be a server that provides cloud computing services. Such a server has been described in detail above and will not be described herein.

Alternatively, for example, in the embodiment of FIG. 1 (c), at least one of the electronics shown in FIG. 1 (c) may be implemented as the sleep environment adjustment device (400).

FIG. 10 depicts an exemplary block diagram of a sleep environment adjustment device related to one embodiment of the present disclosure.

As shown in FIG. 10, a sleep environment adjustment device (400) may include a receiving module (410) and a transmitting module (420).

According to one embodiment of the present disclosure, the sleep environment adjustment device (400) may include a transmitting module (420) that transmits a wireless signal and a receiving module (410) that receives the transmitted wireless signal. In one embodiment, the wireless signal may refer to an orthogonal frequency division multiplexed signal. For example, the wireless signal may be a Wi-Fi based OFDM sensing signal. Further, the transmitting module (420) of the present invention may be implemented through a laptop, smartphone, table PC, smart speaker (AI speaker) or the like, and the receiving module (410) may be implemented through a Wi-Fi receiver. According to embodiments, the receiving module (410) may be implemented through various computing devices, such as a laptop, smartphone, tablet PC, etc. For example, the transmitting module (420) and receiving module (410) may be equipped with wireless chips that follow Wi-Fi 802.11n, 802.11ac, or other standards that support OFDM. In other words, the sleep environment adjustment device (400) may be implemented to acquire object state information with high reliability through relatively low-cost equipment.

In one embodiment, the transmitting module (420) may transmit a wireless signal in one direction in one direction where the object is located. The receiving module (410) may be disposed at a predetermined separation distance from the transmitting module (420), and may receive the transmitted wireless signals from the transmitting module (420). Such wireless signals may be transmitted or received through a plurality of subcarriers as they are orthogonal frequency division multiplexed signals.

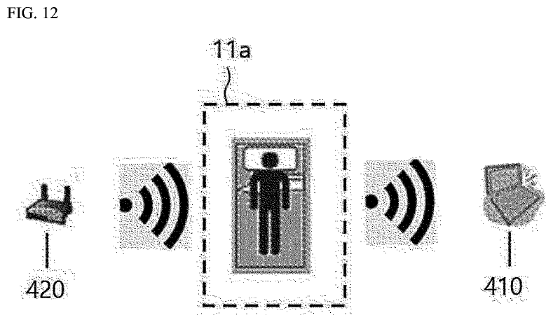

Such transmitting module (420) and receiving module (410) may be equipped to have a predetermined separation distance. In this case, the predetermined separation distance may refer to the space in which the object is active or located. In a specific embodiment, the transmitting module (420) and the receiving module (410) may be characterized as being equipped with positions opposite each other relative to the preset area. Here, the preset area (11a) may be, for example, an area about a location where the user sleeps, such as the area where the bed is located, as shown in FIG. 12. Alternatively, for example, it may refer to an area in which object state information may be acquired, for example, information about the user's movements and/or breathing. The object state information is not limited to information about the user's movement or breathing, but may include sound information about the user, visual information, etc.

The transmitting module (420) and the receiving module (410) may be equipped on each of two sides centered on the bed where the user sleeps. In this case, the sleep environment adjustment device (400) of the present invention is based on Wi-Fi-based OFDM transmitted and received through the transmitting module (420) and the receiving module (410). The sleep environment adjustment device (400) of the present invention may acquire object status information such as information about whether the user is located in a preset area and information about the user's movement or breathing.

According to one embodiment, the transmitting module (420) and the receiving module (410) may transmit and receive wireless signals (e.g., OFDM signals) through one or more antennas. For example, if the transmitting module (420) and the receiving module (410) are each equipped with three antennas, channel status information related to a total of 192 (i.e., 3'63) channels may be acquired every frame through the three antennas and 64 subcarriers. The above specific numerical description of antennas and subcarriers is by way of example only, and the present disclosure is not limited thereto.

According to one embodiment, the transmitting module (420) and the receiving module (410) may be equipped in plurality. In a more specific example, each of the three transmitting modules and four receiving modules may be spaced apart by a predetermined separation distance. In this case, the wireless signals transmitted and received by each of the plurality of transmitting modules and receiving modules may be different.

In an embodiment, the wireless signal received through the receiving module (410) may be a wireless signal that has passed through a channel corresponding to the preset area which may include information indicative of the characteristics of the channel. The receiving module (410) may acquire channel status information form the wireless signal. The channel state information is information indicative of a characteristics of the channel regarding the space in which the user is located. It may be characterized as being calculated based on the wireless signal transmitted module (420) and the wireless signal received through the receiving module.

Specifically, the wireless signal transmitted from the transmitting module (420) may pass through a particular channel (i.e., the space in which the user is located) and be received through the receiving module (410). In this case, the wireless signal may have been transmitted over a plurality of subcarriers, each corresponding to a multi-path. Accordingly, the wireless signal received through the receiving module (410) may be a signal reflecting the movement of the user in the preset area (11a). The processor may acquire channel state information regarding channel characteristics experienced while the wireless signal passes through the channel (i.e., the space in which the user is located) through the received wireless signal. Such channel state information may comprise amplitude and phase. In other words, the sleep environment adjustment device (400) may acquire channel state information regarding characteristics of the space (i.e., the preset area) between the transmitting module (420) and the receiving module (410) based on the wireless signal transmitted from the transmitting signal (420) and the wireless signal received through the receiving module (410) (i.e., the signal reflecting the movement of the object).

According to an embodiment, the receiving module (410) may be characterized as detecting the user's movement based on the received wireless signal when it receives the transmitted wireless signal from the transmitting module (420). The receiving module (410) may acquire information regarding whether the user is located in the preset area through changes in channel state information. According to embodiments, in the process of transmitting and receiving wireless signals through the transmitting module (420) and the receiving module (410), the acquired channel state information may differ when the user is or is not located between the transmitting module (420) and the receiving module (410). According to specific embodiments, the transmitting module (420) and the receiving module (410) may be disposed such that the difference in channel state information acquired in response to the user being located and not located within a region between the transmitting module (420) and the receiving module (410) (i.e., the preset region) is maximized. According to a further embodiment, a directional patch antenna may be provided in response to each of the transmitting module (420) and the receiving module (410). Here, the directional patch antenna may be an antenna module comprising m×n patches (i.e., a number of m horizontal patches and a number of n vertical patches). For example, the antenna pre-beam may be set to increase the difference in signal when the user is located between the transmitting module (420) and the receiving module (410), or when the user is not located. The beam width of the antenna may be preset to be optimal, and the transmitting module (420) and the receiving module (410) may be disposed such that the user is lying in the direction of transmitting and receiving signals using these directional patch antennas. In other words, a wireless link with a direct line-of-sight may be formed between the directional patch antennas of each of the transmitting module (420) and the receiving module (410). This configuration allows the antennas of each module to operate as directional antennas to form a wireless link corresponding to a smaller area (e.g., a preset area).

In other words, a wireless link may be formed between the antennas of transmitting module (420) and the receiving module (410). If the user is located between these wireless links, the user's body may interfere with the wireless links. As a result, the wireless link is distorted and the signal level (i.e., channel state information) varies significantly. In embodiments, changes in signal level may be detected through changes in the Received Signal Strength Indicator (RSSI) and Channel State Information (CSI). Accordingly, the receiving module (410) may use whether the user is located in the preset area (11a) from these changes.

In embodiments, the information regarding whether the user is located in the preset area (11a) may be utilized to determine whether to activate the environment adjustment component (415) or to identify the user's sleep intentions.

According to one embodiment of the present disclosure, the receiving module (410) may calculate the user's sleep state information and adjust the user's sleep environment based on the sleep state information. Specifically, the receiving module (410) may acquire sleep state information regarding whether the user is before, during or after sleep based on the acquired sensing information. The receiving module (410) may adjust the sleep environment of the space where the user is located based on the sleep state information. For a specific example, if the receiving module (410) acquires sleep state information indicating that the user is before sleep, the receiving module (410) may generate environment adjustment information regarding light intensity and illumination (e.g., 3000K white light, 30 lux illumination) to induce sleep based on the sleep state information. Further, the receiving module (410) may adjust the light intensity and illuminance of the space in which the user is located to an appropriate intensity and illuminance for inducing sleep (e.g., 3000K white light at 30 lux) based on the environment adjustment information regarding the light intensity and illuminance for inducing sleep.

In addition, if the sleep state information is acquired before the user sleeps, the receiving module (410) may generate environment adjustment information for controlling the smart home appliance from a time when the user is predicted to be preparing to sleep (e.g., a sleep induction time) to a time when the user falls asleep (i.e., a time when the second sleep state information is acquired).

Specifically, the environment adjustment information may be generated to remove fine dust and harmful gases in advance by a predetermined time (e.g., 20 minutes before the user sleeps), control the indoor temperature and humidity to be optimized according to the season or user, control the illumination, etc.

In addition, the environment adjustment information may include information such as controlling various smart home appliance to cause noise (white noise) at a level that may induce sleep just before sleep, adjusting the blowing intensity to a preset level or less, reducing the intensity of LEDs, or switching from direct wind to indirect wind.

The first environment adjustment information may also include control information that causes to adjust at least one of various environments, such as personalized room temperature, room humidity, blower intensity or noise, according to the operation of the smart home appliance and the acquired sleep state (e.g., sleep quality).

The foregoing specific descriptions of sleep state information and environment adjustment information are by way of example only, and the present invention is not limited thereto.

FIG. 11 (a) depicts an exemplary block diagram of the receiving module and the transmitting module related to one embodiment of the present disclosure.

As shown in FIG. 11 (a), the receiving module (410) may include a network unit (411), a memory unit (412), a sensor unit (413), a sound collection unit (414), an environment adjustment unit (415), and a receiving control unit (416). The receiving module (410) is not limited to the foregoing components, i.e., according to the implementation of the embodiments of the present disclosure, additional components may be included or some of the foregoing components may be omitted.

According to one embodiment of the disclosure, the transmitting module (420) may include a transmitting unit (421) that transmits a wireless signal and a transmitting control unit (422) that controls the operation of transmitting the wireless signal of the transmitting unit (421), as shown in FIG. 11 (a). In embodiments, the transmitting control unit (422) may determine when a wireless signal when a wireless signal is transmitted through the transmitting unit (421). For example, the transmitting control unit (422) may control the transmitting unit (421) in response to when the sleep measurement mode is initiated, thereby transmitting the wireless signal.

According to one embodiment of the present disclosure, the receiving module (410) may include the transmitting module (420), the user terminal (10) and the network unit (411) that transmits and receives data with the external server (20). The network unit (411) may transmit and receive data or the like for performing a sleep environment adjustment method according to one embodiment of the present disclosure with another computing device, server etc. In other words, the network unit (411) may provide a communication function between the receiving module (410), the transmitting module (420), the user terminal (10) and the external server (20). For example, the network unit (411) may receive sleep test records and electronic health records for a plurality of users from a hospital server. In another example, the network unit (411) may receive sound information from the user terminal (10) about the space in which the user is active. In another example, the network unit (411) may transmit environment adjustment information to the environment adjustment unit (415) to adjust the environment of the space in which the user is located. Further, the network unit (411) may allow information to be communicated between the sleep environment adjustment device (400) and the user terminal (10) and the external server (20) by calling a procedure to the sleep environment adjustment device (400).

The network unit (411) according to one embodiment of the present disclosure may comprise any of the various wired and wireless communication systems described above, or a combination thereof.

According to one embodiment of the present disclosure, the memory (412) may store a computer program for performing a sleep environment adjustment method based on the sleep state information according to one embodiment of the present disclosure. The stored computer program may be read and operated by the receiving control unit (416). Further, the memory (412) may store any form of information generated or determined by the receiving control unit (416) and any form of information received by the network unit (411). Furthermore, the memory (412) may store data about the user's sleep. For example, the memory (412) may temporarily or permanently store input/output data (e.g., sound information about the user's sleep environment, sleep state information corresponding to the sound information, or environment adjustment information based on the sleep state information).

According to one embodiment of the present disclosure, the memory (412) may include at least one type of storage medium at least one type of storage medium such as a flash memory type, hard disk type, a multimedia card micro type, a card type memory (e.g., SD or XD memory, etc.), random access memory, static random access memory (SRAM), read-only memory (ROM), electrically erasable programmable read-only memory (EEPROM), programmable read-only memory (PROM), magnetic memory, magnetic disk, and optical disk. The sleep environment adjustment device (400) may also operate related to web storage that performs the storage function of the memory (412) over the internet. The foregoing description of memory is by way of example only, and the present invention is not limited thereto.

The computer program, when loaded into the memory (412), may include one or more instructions that cause the receiving control unit (416) to perform methods/operations according to various embodiments of the present disclosure. In other words, the receiving control unit (416) may execute the one or more instructions to perform methods/operations according to various embodiments of the present disclosure.

According to one embodiment of the present disclosure, the receiving module (410) may include the sensor unit (413) that acquires one or more sensing information about a space. In the present invention, a space may refer to a space in which a user lives, for example, bedroom in which the user sleeps.

According to an embodiment, the sensor unit (413) may include a first sensor unit that detects movement of a user in a space. The first sensor part may comprise at least one of a passive infrared sensor (PIR sensor) and an ultrasonic sensor. The PIR sensor may detect changes in infrared radiation emitted by the user's body to detect movement of the user within a detection range. For example, the PIR sensor may identify infrared light emitted by the user's body in the range of 8 μm to 14 μm to detect the user's movement within the bedroom. Ultrasonic sensors may detect object movement by generating sound waves and detecting the signals that bounce back certain objects. For example, an ultrasonic sensor generates sound waves within a bedroom space. As the user enter the bedroom, the ultrasonic sensor may detect the movement of the user inside the bedroom through the sound waves reflecting off the user's body.

In addition, in an embodiment, the sensor unit (413) may include a second sensor part that detects whether the user is located in a preset area of a space based on the wireless signal. The second sensor part may receive a wireless signal transmitted from the transmitting module (420) and detect whether the user is located in the preset area based on the received wireless signal. In an embodiment, the preset area relates to an area located within a space where a user lies down to sleep. For example, it may refer to an area equipped with a bed. As a specific example, in the present invention, a space may refer to a space inside a bedroom, and the preset area may refer to a space where a bed is located.

In embodiments, the second sensor part may be characterized as being disposed opposite the transmitting module (420) and each other based on the preset area. For example, the transmitting module (420) and the second sensor part may be disposed on each of two sides centered on a bed in which the user sleeps. In this case, the sleep environment adjustment device (400) of the present invention may acquire information regarding whether the user is located in a preset area based on Wi-Fi-based OFDM signals transmitted and received through the transmitting module (420) and the receiving module (410) and object state information which is information regarding the user's movement and/or breathing.

According to one embodiment, the receiving module (410) may permit operation of the environment adjustment unit (415) when it is identified through the second sensor part that the user is located in the preset area. In other words, the receiving module (410) may allow the environment adjustment unit (415) to operate only when the user is detected to be located in the preset area (11a). In other words, the receiving module (410) may control the operation of the environment adjustment unit (415) to perform the environment adjustment operation by generating environment adjustment information only when the user is located in the preset area. If the user is not located at a particular area, the environment adjustment unit (415) may not perform actions to change the sleep environment.

In further embodiments, the sensor unit (413) may include one or more environment sensing modules for acquiring room environment information related to at least one of a user's body temperature, room temperature, room airflow, room humidity and room illumination related to the user's sleep environment. The room environment information is information related to a sleep environment of the user. The room environment information may be information that serves as a baseline for considering the impact of external factors on the user's sleep through a sleep state regarding changes in the user's sleep stages. The one or more environment sensing modules may include at least one of a sensor module, for example, a temperature sensor, an airflow sensor, a humidity sensor, a sound sensor, and a light sensor. However, it is not limited thereto, and may further include a variety of other sensors capable of measuring the external environment that may affect a user's sleep.

According to one embodiment of the present disclosure, the receiving module (410) may include a sound collection unit (414). The sound collection unit (414) may comprise a small microphone module and may acquire information about sound in a space where the user sleeps. According to an embodiment, the microphone module in the sound collection unit (414) may comprise micro-electro mechanical systems (MEMS) that are relatively small in size. Such microphone modules are cost advantageous and may be manufactured in a very small size. However, they may have a lower signal-to-noise ratio (SNR) compared to a condenser microphone or dynamic microphone. A low signal-to-noise ratio may mean that the ratio of a sound to be identified to noise, which is a sound not to be identified, is high, which means that it is not easy to identify the sound (i.e., unclear). The information subject to analysis in the present invention may be sound information related to a user's breathing and movements acquired during sleep, i.e., sleep sound information. Such sleep sound information is information about very subtle sounds, such as the user's breathing and movements, and is acquired along with other sounds during the sleep environment. Therefore, it may be very difficult to detect and analyze if it is acquired through a microphone module as described above with a low signal-to-noise ratio. Accordingly, when the sleep sound information having a low signal-to-noise ratio is acquired, the receiving control unit (416) may process it into data for process and/or analysis.

According to one embodiment of the present disclosure, the receiving module (410) may include an environment adjustment unit (415). The environment adjustment unit (415) may adjust the user's sleep environment. Specifically, the environment adjustment unit (415) may adjust at least one of air quality, illumination, temperature, wind direction, humidity, and the sound of the space in which the user is located based on the environment adjustment information. The environment adjustment information may be a signal generated by the receiving control unit (416) based on a determination of the user's sleep state information. For example, the environment adjustment information may include information about lowering or increasing light levels, etc. In a more specific example, the environment adjustment information may include control information to gradually increase 3000K white light from 0 lux to 250 lux illumination starting 30 minutes before a predicted wake-up event. As a further example, the environment adjustment information may include control information for adjusting at least one of temperature, humidity, wind direction or sound. The environment adjustment information may include various information related to removing fine dust, removing harmful gases, activating allergy care, activating deodorization/sterilization, adjusting room temperature, adjusting dehumidification, adjusting humidification, adjusting blowing intensity, selecting and adjusting wind direction, adjusting operating noise, adjusting vibration, adjusting LED lighting, etc. based on the real-time sleep status of the user. The foregoing specific description of the environment adjustment information is by way of example only, and the present invention is not limited thereto.

The environment adjustment unit (415) may perform control of at least one of light control, temperature control, wind control, humidity control and sound control. However, it is not limited thereto, and the environment adjustment unit (415) may further perform various control operations that may result in changes to the user's sleep environment. In other words, the environment adjustment unit (415) may adjust the user's sleep environment by performing various control actions based on environment adjustment signals from the receiving control unit (416).

In a further embodiment, the environment adjustment unit (415) may be implemented through connection via the Internet of Things (IoT). Specifically, the environment adjustment unit (415) may be implemented in connection with various devices that may change the indoor environment related to the space in which the user is located. For example, the environment adjustment unit (415) may be implemented with various smart home appliances such as smart air conditioners, smart heaters, smart air purifiers, smart boilers, smart windows, smart humidifiers, smart dehumidifiers, and smart lights based on connection via the Internet of Things. The foregoing specific descriptions of the environment adjustment unit (415) are by way of example only, and the present invention is not limited thereto.

According to one embodiment of the present disclosure, the receiving control unit (416) may comprise one or more cores, and may include a processor for data analysis such as a central processing unit (CPU) of a computing device, a general purpose graphics processing unit (GPGPU), a tensor processing unit (TPU) etc., and deep learning.

The receiving control unit (416) may read a computer program stored in the memory (412) to perform data processing for machine learning according to one embodiment of the present disclosure.

The receiving control unit (416) may perform operations for training a neural network, according to one embodiment of the present disclosure. The receiving control unit (416) may perform computation for training a neural network, such as processing input data for training in deep learning (DL), extracting features from the input data, calculating errors and updating weights of the neural network using backpropagation etc.

In addition, at least one of the CPU, GPGPU, and TPU of the receiving control unit (416) may process the training of the network function. For example, the CPU and the GPGPU may together process learning a network function process learning a network function and classifying data using the network function. Further, in one embodiment of the present disclosure, processors of a plurality of computing devices may be used together to process the learning of the network function and the classification of data using the network function, Further, a computer program executed on a computing device according to one embodiment of the present disclosure may be a CPU, GPGPU, or TPU executable program.

In the present disclosure, a network function may be used interchangeably with an artificial neural network and a neural network. In the present disclosure, the network function may include one or more neural networks, in which case the output of the network function may be an ensemble of the outputs of the one or more neural networks.

In the present disclosure, a model may include a network function. The model may also include one or more network functions, in which case the output of the model may be an ensemble of the outputs one or more network functions.

The receiving control unit (416) may read a computer program stored in the memory (412) to provide a sleep analysis model according to one embodiment of the present disclosure. According to one embodiment of the present disclosure, the receiving control unit (416) may perform calculations to produce environment adjustment information based on the sleep state information. According to one embodiment of the disclosure, the receiving control unit (416) may perform calculations to train the sleep analysis model.

According to one embodiment of the present disclosure, the receiving control unit (416) may generally handle the overall operation of the sleep environment adjustment device (400). The receiving control unit (416) may process signals, data, information etc. that are input or output through the components discussed above, or operate applications stored in the memory (412). In doing so, it may provide or process the appropriate information or function to the user terminal.

According to one embodiment of the present disclosure, the receiving control unit (416) may acquire sound information about the space in which the user sleeps. According to one embodiment of the disclosure, acquisition of the sound information may be acquiring or loading the sound information stored in the memory (412). Further, acquiring the sound information may be receiving or loading data from another storage medium, another computing device, a separate processing module within the same computing device based on wired or wireless communication means.

According to one embodiment, the receiving control unit (416) may acquire the sleep sound information from the living environment sound information. Here, the living environment sound information may be sound information acquired in the user's daily life. For example, the living environment sound information may include various sound information acquired according to the user's life such as sound information related to cleaning, sound information related to cooking food, sound information related to watching TV, etc.

Specifically, the receiving control unit (416) may identify singularities in the living environment sound information where information of a preset pattern is detected. Here, the preset pattern information may be related to breathing and movement patterns related to sleep. For example, in the wake state, the breathing pattern may be irregular and the body movement may be high because all nervous systems are active. Also, the throat muscles are not relaxed, so breathing sounds may be very low. On the other hand, when the user is sleeping, the autonomic nervous system is stabilized, the breathing pattern may become more regular, the body movements may become less frequent and the breathing sounds may become louder. In other words, the receiving control unit (416) may identify as a singularity a point in the living environment sound information when a preset pattern of sound information related to regular breathing, less body movement or less breath sound is detected. Further, the receiving control unit (416) may acquire the sleep sound information based on the living environment sound information acquired based on the identified singularity. The receiving control unit (416) may identify a singularity related to the time of the user's sleep in the time-series acquired living environment sound information, acquire the sleep sound information based on the singularity.

In a specific example, the receiving control unit (416) may identify a singularity related to a time when the preset pattern is identified in the living environment sound information. Furthermore, the receiving control unit (416) may acquire the sleep sound information based on the sound information acquired after the singularity based on the identified singularity.

In other words, by identifying a singularity regarding the user's sleep from the living environment sound information, the receiving control unit (416) may extract and acquire only the sleep sound information from a large amount of sound information. In other words, only the sound related to sleep (i.e., sleep sound information) may be acquired from the sound generated in a space. This may provide convenience by allowing the user to automate the process of recording sleep time, while also contributing to improve the accuracy of the acquired sleep sound information.

According to an embodiment, the receiving control unit (416) may calculate sleep state information based on the sound information. Specifically, the receiving control unit (416) may calculate the sleep state information based on the sleep sound information of the user acquired through the sound collection unit (414).

In one embodiment, the sleep state information may include information regarding whether the user is sleeping. Specifically, the sleep state information may include at least one of a first sleep state information indicating that the user is before sleep, a second sleep state information indicating that the user is during sleep, and a third sleep state information indicating that the user is after sleep. In other words, if the first sleep state information is acquired regarding the user, the receiving control unit (416) may determine that the user is in pre-sleep state (i.e., before bedtime). If the second sleep state information is acquired, the receiving control unit (416) may determine that the user is in a sleeping state. And if the third sleep state information is acquired, the receiving control unit (416) may determine that the user is in a post-sleep state (i.e., wake-up).

Such sleep state information may be characterized as being acquired based on sleep sound information. The sleep sound information may include sound information acquired during the user's sleep in a space where the user is located in a non-contact manner.

According to an embodiment, the receiving control unit (416) may calculate the sleep state information based on the collected sound information (S140). In an embodiment, the receiving control unit (416) may acquire sleep state information related to whether the user is before sleep or during sleep based on a singularity identified from the sound information. Specifically, the receiving control unit (416) may determine that the user is before sleeping if no singularity is identified. And the receiving control unit (416) may determine that the user is sleeping after the singularity if the singularity is identified. Further, the receiving control unit (416) may identify a time point (e.g., a wake-up time point) after the singularity is identified where the preset pattern is not observed, and if the time point is identified, the receiving control unit (416) may determine that the user is after sleep, i.e., has woken up.

In other words, the receiving control unit (416) may acquire sleep state information regarding whether the user is before, during, or after sleep based on whether the singularity is identified in the sound information and whether the preset pattern is continuously detected after the singularity is identified.

According to one embodiment of the present disclosure, the receiving control unit (416) may generate environment adjustment information based on the sensing information and the sleep state information. Specifically, the receiving control unit (416) may generate the sensing information acquired through the sensor unit (413) and the environment adjustment information based on sleep state information acquired as a result of sound analysis. By generating the environment adjustment information based on the sensing information and the sleep state information, and transmitting the generated environment adjustment information to the environment adjustment unit (415), the receiving control unit (416) may control may control the sleep environment change operation of the environment adjustment unit (415).

In an embodiment, the receiving control unit (416) may generate the environment adjustment information based on sleep state information. The sleep state information is information about whether the user is sleeping. The sleep state information may include at least one of first sleep state information that the user is before sleep, second sleep state information that the user is during sleeping, and third sleep state information that the user is after sleeping.

In more detail, the receiving control unit (416) may generate the first environment adjustment information based on the first sleep state information. Specifically, if the receiving control unit (416) acquires the first sleep state information that the use is before sleeping, the receiving control unit (416) may generate the first environment adjustment information based on the first sleep state information. In other words, the receiving control unit (416) may generate the first environment adjustment information to provide the preset white light for a certain period of time if the user's sleep state is before sleeping.

According to an embodiment, the sleep induction time may be determined by the receiving control unit (416). Specifically, the receiving control unit (416) may determine the sleep induction time by exchanging information with the user terminal (10) of the user. For example, a user may generate sleep plan information by setting a time at which the user wishes to sleep and a time at which the user wishes to wake up through the user terminal (10), and may transmit the generated sleep plan information to the receiving control unit (416). In this case, the sleep plan information may include the desired bedtime information and the desired wake-up time information. The receiving control unit (416) may identify a time to induce sleep based on the desired bedtime information. For example, the receiving control unit (416) may determine that the sleep induction time is 20 minutes before the time when the user wants to sleep (i.e., the desired bedtime). As a specific example, if the user's set desired time to sleep 11:00, the receiving control unit (416) may identify 10:40 as the sleep induction time. The specific numerical description of the above time point is by way of example only, and the present invention is not limited thereto.

Further, according to an embodiment, the receiving control unit (416) may acquire sleep intent information of the user based on the living environment sound information, and determine a sleep induction time based on the sleep intent information. The sleep intention information may be a quantitative numerical representation of the user's intention to sleep. For example, the higher intention to sleep of the user may result in a sleep intention information close to 10, and the lower intention to sleep may result in a sleep intention information close to 0. The above specific numerical description of the sleep intention information is by way of example only, and the present invention is not limited thereto.

The receiving control unit (416) may acquire the sleep intention information based on the living environment sound information. According to one embodiment, the receiving control unit (416) may calculate the sleep intent information based on the number of identified types of sound. Further, the receiving control unit (416) may calculate the sleep intent information based on the number of identified sound types. The receiving control unit (416) may calculate the sleep intent information based on the number of identified sound types, such that the higher number of sound types results in the lower sleep intent information, and the lower number of sound types results in the higher sleep intent information. For a specific example, if the living environment sound information includes three types of sound (e.g., a vacuum cleaner sound, a TV sound, and the user voice), the receiving control unit (416) may calculate the sleep intent information as 2 points. Also, for example, if the living environment sound information includes one type of sound (e.g., a washing machine), the receiving control unit (416) may calculate the sleep intent information as 6 points. The foregoing specific numerical descriptions of the types of sounds included in the living environment sound information and the sleep intent information are by way of examples only, and the present invention is not limited thereto.

In other words, the receiving control unit (416) may acquire sleep intention information regarding how much the user intends to sleep based on the number of types of sounds included in the living environment sound information. For example, the more types of sounds are identified, the more sleep intention information (i.e., low score sleep intention information) may be output indicating that the user's sleep intention is low.

In addition, in an embodiment, the receiving control unit (416) may generate an intent score table by matching each of the plurality of sound information with a different intent score dictionary. For example, the first sound information related to a washing machine may be matched with an intent score of 2. The second sound related to a humidifier may be pre-matched with an intent score of 5. And the third sound information related to the voice may be matched may be matched with an intent score of 1. Receiving control unit (416) may pre-match a relatively high intent score for sound information related to the user's sleep (e.g., sounds generated by the user's activity such as a vacuum cleaner, washing dishes, voice sounds, etc.). The receiving control unit (416) may generate an intent score table by pre-matching relatively low intent scores for sound information that is not related to the user's sleep (e.g., sound unrelated to the user's activity such as vehicle noise, rain, etc.). The specific numerical description of the intent scores matched to each of the above sound information are by way of examples only, and the invention is not limited thereto.

The receiving control unit (416) may acquire the sleep intent information based on the living environment sound information and the intent score table. Specifically, in response to a time when at least one of the plurality of sounds included in the intention score table is identified in the living environment sound information, the receiving control unit (416) may record an intention score matched to the identified sound. As a specific example, if a vacuum cleaner sound is identified in response to the first time point during the during the real-time acquisition of the living environment sound information, the receiving control unit (416) may record intent scores of two matched to the vacuum cleaner sound in response to the first time point. As each of the various sounds is identified in the process of acquiring the living environment sound information, the receiving control unit (416) may record an intent score matched to the identified sound at the corresponding time point.

In an embodiment, the receiving control unit (416) may acquire sleep intent information based on the sum of the intent scores acquired over a predetermined period of time (e.g., 10 minutes). For example, the higher intention score acquired during the 10 minutes may result in the higher sleep intention information, and the lower intention score acquired during the 10 minutes may the lower sleep intention information. The foregoing specific numerical descriptions of predetermined time period are by way of example only, and the present invention is not limited thereto.

In other words, the receiving control unit (416) may acquire sleep intention information regarding how much the user intends to sleep based on the characteristics of sound included in the living environment sound information. For example, as sound related to the user's activity are identified, sleep intention information indicating that the user's intention to sleep is low (i.e., low-scoring sleep intention information) may be output.

According to an embodiment, the receiving control unit (416) may determine a time to induce sleep based on the based on the sleep intent information. Specifically, the receiving control unit (416) may identify a time when the sleep intent information exceeds a predetermined threshold score as a time to induce sleep. In other words, the receiving control unit (416) may identify that when high sleep intent information is acquired, it is an appropriate time to induce sleep, i.e., the sleep induction time.

In addition, in an embodiment, the receiving control unit (416) may calculate the sleep intention weighted information based on the sensing information acquired through the sensor unit (413). Specifically, the receiving control unit (416) may determine that the user has a high intention to sleep if the first sensor identifies that the user is located in the preset area through the second sensor after the user's movement in a space occurs, and may calculate the sleep intention weighted information related to 1 in response. If the receiving control unit (416) detects through the first sensor unit and the second sensor unit that there is no movement of the user is a space and the preset area, and that user is not located, the receiving control unit (416) may determine that the user does not have a sleep intention, and may calculate the sleep intention weighted information related to 0. In other words, if the receiving control unit (416) detects through the sensor unit (413) that the user is located in a certain space (e.g., a bed space), it may calculate the sleep intention weighted information related to 1. If the receiving control unit (416) detects that the user is not located in the particular space, it may calculate sleep intent weighted information related to 0. In other words, the receiving control unit (416) may calculate sleep intent weighted information related to 0 or 1 depending on whether the user is located in a space and the preset area.

According to an embodiment, the receiving control unit (416) may determine a sleep induction time based on the sensing information and the sleep state information. Specifically, the receiving control unit (416) may determine a sleep induction time based on sensing information acquired through the sensor unit (413) and sleep state information acquired as a result of sound analysis. The receiving control unit (416) may determine the sleep induction time based on the sleep intention information calculated based on the living environment sound information and the sleep intention weighted information calculated through the sensing information. For example, the final sleep intention information may be acquired from the sleep intention information and the sleep intention weighted information, and a time when the final sleep intention information exceeds a certain threshold may be determined as a sleep induction time. For example, the receiving control unit (416) may calculate the final sleep intention information through a multiplication of the sleep intention information and the sleep intention weighted information. As a specific example, if the sleep intention information calculated based on the living environment sound information is “9” and the sleep intention weighted information calculated based on the sensing information is “0”, the final sleep intention information may be calculated as 0. As a result, the receiving control unit (416) may determine that the sleep intention information does not exceed a predetermined threshold (e.g., 8). In another example, if the sleep intent information is “9” and the sleep intent weighted information is “1”, the final sleep intent information may be calculated as 9. As a result, the receiving control unit (416) may determine that a predetermined threshold (e.g., 8) is exceeded, and may determine that time as a sleep induction time. The specific numerical descriptions of the foregoing sleep intention information, the sleep intention weighted information and the final sleep intention information are by way of examples only, and the present invention is not limited thereto. As described above, even if high sleep intent information is acquired from the sound information, the final sleep intent information may change depending on whether the user is located at a certain location. For example, even if a high sleep intention information (e.g., 10) is calculated based on the living environment sound information, if the user is not located at a certain location, the final sleep intention information becomes 0. Accordingly, it may be determined that the user's sleep intent is low.

As described above, the receiving control unit (416) may determine a sleep induction time of the user. Accordingly, when the receiving control unit (416) acquires the first sleep state information that the user is before sleep, the receiving control unit (416) may generate the first environment adjustment information (supplying 3000K white light at an illuminance of 30 lux) to adjust the light to a time when the second sleep state information is acquired based on the sleep induction time.

In other words, if the user's state is a pre-sleep state, the receiving control unit (416) may generate the first environment adjustment information that causes the light to be adjusted from a time when the user is predicted to be preparing to sleep (e.g., the time of sleep induction) to a time when the user falls asleep (i.e., the time when the second sleep state information is acquired). The receiving control unit (416) may determine to transmit the corresponding first environment adjustment information to the environment adjustment unit (415).

According to one embodiment of the present disclosure, the receiving control unit (416) may generate the second environment adjustment information based on the second sleep state information. The second environment adjustment information may be control information to minimize illumination to adjust a dark room environment without light. In other words, the receiving control unit (416) may minimize illumination to adjust a dark room environment without light when the user is sleeping.

In other words, when the receiving control unit (416) detects that the user enters sleep (or a sleep stage) (when the second sleep state information is acquired), the receiving control unit (416) may generate control information, i.e., second environment adjustment information, to prevent light from being provided. Accordingly, the probability of the user falling into a deep sleep may be increased, thereby improving the quality of sleep.

According to one embodiment of the present disclosure, the receiving control unit (416) may generate the third environment adjustment information based on wake-up induction time.

In other words, the receiving control unit (416) may generate third environment adjustment information, such as to provide a gradually increasing intensity of white light from the wake-up induction time to the wake-up time of the user, if the user's sleep state is sleeping.

In one embodiment, the wake-up induction time may be characterized as being determined based on the desired wake-up time information.

The desired wake-up time information may be information about when the user wants to wake up. For example, the desired wake-up time information acquired from the first user may relate to 7:00 AM. The above specific description of the wake-up prediction time is by way of example only, and the present invention is not limited thereto.

In one embodiment, the desired wake-up time information may be characterized as being acquired through information exchange with the user terminal (10) of the user. The user may set a time at which the user wishes to go to bed and a time at which the user wishes to wake up through the user terminal (10) and transmit it to the receiving control unit (416). The receiving control unit (416) may acquire the desired wake-up time information based on the wake-up time set by the user of the user terminal (10).

In another embodiment, the wake-up induction time may be determined based on the wake-up prediction time. Here, the wake-up prediction time may be characterized in that it is determined based on a sleep entering time identified through the second sleep state information. Specifically, the receiving control unit (416) may determine the user's sleep entering time from the second sleep state information indicating that the user is sleeping. The receiving control unit (416) may determine the wake-up prediction time based on the sleep entering time identified through the second sleep state information. For example, the receiving control unit (416) may determine the wake-up prediction time as a time after 8 hours, which is an appropriate sleep time, based on the sleep entering time. As a specific example, if the user enters sleep at 11:00 p.m., the receiving control unit (416) may determine the wake-up prediction time to be 7:00 a.m. The specific numerical description of each of the above time point is by way of example only, and the present invention is not limited thereto. In other words, the receiving control unit (416) may determine the wake-up prediction time point based on the time the user goes to sleep.

In another embodiment, the wake-up prediction time may be characterized as being determined based on the user's sleep stage information. For example, the user may wake up most refreshed if they wake up in the REM stage. During a night's sleep, the user may have a sleep cycle of light sleep, deep sleep, light sleep and REM sleep. Waking up in the REM sleep stage is the most refreshed.

Accordingly, the receiving control unit (416) may determine a wake-up prediction time for the user through the sleep stage information regarding the user's sleep stage. In a specific example, the receiving control unit (416) may determine a wake-up prediction time through the sleep stage information to be when the user transitions from the REM stage to another sleep stage. In other words, the receiving control unit (416) may determine a wake-up prediction time based on the sleep stage information when the user is most refreshed (i.e., the REM sleep stage).

As described above, the receiving control unit (416) may determine the user's wake-up prediction time based on at least one of the sleep plan information, the sleep entering time and the sleep stage information acquired from the user terminal. Further, when the receiving control unit (416) determines the wake-up prediction time, which is a time when the user wants to wake up, the receiving control unit (416) may determine a wake-up induction time based on the wake-up prediction time. For example, the receiving control unit (416) may determine the wake-up induction time as a time 30 minutes before the time the user want to wake up. In a specific example, if the user's desired wake-up time (i.e., wake-up prediction time) is 7:00 a.m., the receiving control unit (416) may determine 6:30 a.m. as the wake-up induction time. The above specific description of the time is by way of example only, and the present invention is not limited thereto.

In other words, the receiving control unit (416) may identify the wake-up induction time by determining a wake-up prediction time when the user is expected to wake up, and generate third environment adjustment information to provide 3000K white light from the wake-up induction to the wake-up time (e.g., until the user actually wakes up) at a gradually increasing illuminance from 0 lux to 250 lux. The receiving control unit (416) may determine to transmit the third environment adjustment information to the environment adjustment unit (415). Accordingly, the environment adjustment unit (415) may perform adjustment operations related to light in the space where the user is located based on the third environment adjustment information. For example, the environment adjustment unit (415) may gradually increase the illuminance of 3000K white light from 0 lux to 250 lux 30 minutes before waking up.

According to one embodiment of the present disclosure, the receiving control unit (416) may acquire living environment sound information and may acquire sleep sound information based on that sound information.

According to one embodiment of the present disclosure, the receiving control unit (416) may perform preprocessing on the sleep sound information. The preprocessing of the sleep sound information may be a preprocessing of noise removal. Specifically, the receiving control unit (416) may categorize the sleep sound information into one or more sound frames having a predetermined time unit. Further, the receiving control unit (416) may identify a minimum sound frame having a minimum energy level based on an energy level of each of the one or more sound frames. The receiving control unit (416) may perform denoising of the sleep sound information based on the minimum sound frame.

In a specific example, the receiving control unit (416) may categorize 30 seconds of sleep sound information into one or more sound frames of a very short 40 ms size. Further, the receiving control unit (416) may compare the size of each of the plurality of sound frames regarding the 40 ms size to identify the smallest sound frame with the minimum energy level. The receiving control unit (416) may remove the identified minimum sound frame component from the entire sleep sound information (i.e., 30 seconds of sleep sound information). For example, as the minimum sound frame component is removed from the sleep sound information, preprocessed sleep sound information may be acquired. In other words, the receiving control unit (416) may identify the minimum sound frame as a background noise frame and remove it from the original signal (i.e., the sleep sound information), thereby performing preprocessing for noise removal.

Further, the receiving control unit (416) may generate a spectrogram (300) in response to the sleep sound information (210), as shown in FIG. 6 (a). Here, the sleep sound information (210) may refer to preprocessed sleep sound information. In other words, the receiving control unit (416) may generate the spectrogram in response to the preprocessed sleep sound information. Generation of the spectrogram has been described in detail above and will not be repeated.

According to one embodiment of the present disclosure, a spectrogram generated by the receiving control unit (416) in response to the sleep sound information (210) may include a Mel-Spectrogram. The receiving control unit (416) may acquire the Mel-Spectrogram through a Mel-Filter Bank for the spectrogram.

In general, the human cochlea may have different vibrating regions depending on the frequency of the sound data. Further, the human cochlea is characterized by its ability to detect frequency changes in the lower frequency bands and its inability to detect frequency changes in the higher frequency bands. Accordingly, a Mel-Spectrogram may be acquired from the spectrogram by utilizing a Mel-filter bank to have a recognition ability similar to the characteristics of the human cochlea for sound data. In other words, the Mel-filter bank may apply a small filter bank in a low frequency band and a wider filer bank as the frequency band increases. In other words, the receiving control unit (416) may apply a Mel-filter bank to the spectrogram to recognize sound data to mimic the characteristics of the human cochlea, thereby acquiring a Mel-Spectrogram. The Mel-Spectrogram may include frequency components that reflect human hearing characteristics. In other words, the Mel-Spectrogram is generated in response to the sleep sound information in the present invention, and the spectrogram subject to analysis using the neural network may include the Mel-Spectrogram described above.

Further, the receiving control unit (416) may process the spectrogram (300) as an input to a sleep analysis model to acquire sleep stage information. Here, the sleep analysis model is a model for acquiring sleep stage information related to changes in sleep stages of the user. The sleep stage information may be output using the sleep sound information acquired during the user's sleep as input. In embodiments, the sleep analysis model may comprise a neural network model configured through one or more network functions. The network functions are described in detail above and will not be repeated herein.

As described above, the receiving control unit (416) may acquire a spectrogram based on the sleep sound information. In this case, the conversion to a spectrogram may be to facilitate analysis of breathing or movement patterns regarding relatively small sounds. Further, the receiving control unit (416) may utilize a sleep analysis model comprising a feature extraction and a feature classification model to generate sleep stage information based on the acquired spectrogram. In this case, the sleep analysis model may perform sleep stage prediction using spectrograms corresponding to a plurality of epochs as input so that both past and future information may be considered. Therefore, more accurate sleep stage information may be output.

In other words, the receiving control unit (416) may utilize the sleep analysis model as described above to output sleep stage information corresponding to the sleep sound information. According to an embodiment, the sleep stage information may be information about changing sleep stages during the user's sleep. For example, the sleep stage information may refer to information about the user's change in light sleep, moderate sleep, deep sleep or REM sleep at each time point during the user's last eight hours of sleep. The foregoing specific description of sleep stage information is by way of example only, and the invention is not limited thereto.

According to one embodiment of the present disclosure, the receiving control unit (416) may perform data augmentation based on the preprocessed sleep sound information. This data augmentation is intended to enable the sleep analytics model to robustly output sleep state information (e.g., sleep stage information) from sound measured in different domains (e.g., different bedrooms, different microphones, different placement locations, etc.). In embodiments, data augmentation may include at least one of pitch shifting, gaussian noise, loudness control, dynamic range control and spec augmentation.

According to one embodiment, the receiving control unit (416) may perform data augmentation related to pitch shifting based on the sleep sound information. For example, the receiving control unit (416) may perform data augmentation by adjusting the pitch of sound, such as by increasing or decreasing the pitch of the sound at predetermined intervals.

In addition to pitch shifting, the receiving control unit (416) may perform gaussian noise, which performs data augmentation by correcting for noise, loudness control, which performs data augmentation by correcting for sound so that the sound quality is maintained even when the volume is changed, dynamic range control, which performs data augmentation by adjusting dynamic range, which is logarithmic ratio measured in dB between the maximum amplitude and minimum amplitude of sound, and spec augmentation, which involves increasing the specification of the sound.

In other words, the receiving control unit (416) may perform robust recognition in response to sleep sound acquired from the sleep analysis model in various environment to improve the accuracy of sleep stage prediction through data augmentation of the sound information (i.e., sleep sound information) that is the basis for the analysis of the present invention.

According to one embodiment of the present disclosure, the receiving control unit (416) may acquire the fourth environment adjustment information based on the third sleep state information. Since the fourth environment adjustment information is the same as described in related to the operation of the processor (130) in the embodiment of FIG. 1 (a), a redundant description will be omitted.

According to an embodiment of the present disclosure, the receiving control unit (416) may determine to transmit the environment adjustment information to the environment adjustment unit (415). Specifically, the receiving control unit (415) may generate environment adjustment information about light intensity adjustment, and by determining to transmit the environment adjustment information to the environment adjustment unit (415), the receiving control unit (416) may control the light intensity adjustment operation of the environment adjustment unit (415).

According to embodiments, light or air quality may be one representative factor that may affect sleep quality. For example, the intensity, color, exposure, etc. of light may have a positive or negative effect on sleep quality. Furthermore, the type/concentration of fine dust, the type/concentration of harmful gases, the presence or absence of allergenic substances, the temperature or humidity of the air, etc. also greatly affect the quality of sleep. Accordingly, the receiving control unit (416) may adjust the illumination or air quality to improve the quality of the user's sleep. For example, the receiving control unit (416) may monitor conditions before falling asleep or after falling asleep, and accordingly perform a light adjustment to effectively wake the user up. In other words, the receiving control unit (416) may determine a sleep state (e.g., sleep stage) and automatically adjust the light or air quality to maximize sleep quality.

In one embodiment, the receiving control unit (416) may receive sleep plan information from the user terminal (10). The sleep plan information is information that the user generates through the user terminal (10). The sleep plan information may include, for example, desired bedtime information and desired wake-up time information. The receiving control unit (416) may generate external environment adjustment information based on the sleep plan information. In a specific example, the receiving control unit (416) may identify the user's bedtime from the sleep plan information and generate the environment adjustment information based on the bedtime.

Further, the receiving control unit (416) receives the sleep plan information from the user terminal (10). Based on the sleep plan information, the receiving control unit (416) may generate the first environment adjustment information for controlling the smart home appliance according to an embodiment of the present disclosure from a time when the user is predicted to prepare for sleep (e.g., a sleep induction time) to a time when the user falls asleep (i.e., when the second sleep state information is acquired).

In addition, for example, the receiving control unit (416) may identify a time when the user enters sleep, i.e., a sleep induction time through the second sleep state information, and may generate the second environment adjustment information based on this.

In an embodiment, the receiving control unit (416) may generate the environment adjustment information based on the sleep stage information. In an embodiment, the sleep stage information may include information about changes in the sleep stage of the user acquired time series through analysis of sleep sound information.

In addition, the receiving control unit (416) may generate environment adjustment information to provide appropriate light levels during sleep based on changes in the user's sleep stages.

In addition, for example, the receiving control unit (416) may identify a desired wake-up time of the user from the sleep plan information and generate a wake-up prediction time based on the desired wake-up time. Accordingly, environment adjustment information may be generated.

In addition, the receiving control unit (416) may determine to transmit the environment adjustment information to the environment adjustment unit (415). In other words, the receiving control unit (416) generates environment adjustment information that enables the user to easily fall asleep or naturally wake up when sleeping or wake up based on the sleep plan information. The environment adjustment information may be used to control the environment adjustment operations of the environment adjustment unit (415), thereby improving the quality of the user's sleep.

In a further embodiment, the receiving control unit (416) may generate recommended sleep plan information based on the sleep stage information.

According to one embodiment, the receiving control unit (416) may update the environment adjustment information by comparing the actual wake-up time of the user with the desired wake-up time information.

The receiving control unit (416) performs a comparison of the desired wake-up time information and the actual wake-up time information. If, as a result of the comparison, the respective information differs from each other, the environment adjustment information may be updated. Here, the actual wake-up time information compared to the desired wake-up time information may include information about actual wake-up times accumulated over a certain number of times. For example, the actual wake-up time information may include information about when the user actually wakes up during a week.

In an embodiment, the receiving control unit (416) may analyze the difference between the desired wake-up time and the accumulated actual wake-up time to update the environment adjustment information. Specifically, if the actual wake-up time is later than the desired wake-up time, the receiving control unit (416) may gradually increase the maximum brightness of the white light provided at the time of wake-up to advance the user's circadian rhythm. For example, on the day after the actual wake-up time is later than the desired wake-up time, the environment adjustment information may be updated such that the maximum brightness of the white light supplied at the time of wake-up is higher than the previous day in response to the user's wake-up time. On the other hand, if the actual wake-up time is earlier than the desired wake-up time, the receiving control unit (416) may reduce the maximum brightness of the white light supplied at the wake-up time to delay the user-s wake-up time. For example, the next day when the actual wake-up time is earlier than the desired wake-up time, the environment adjustment information may be updated such that the maximum brightness of the white light supplied at the wake-up time is lower than the previous day in response to the user's wake-up time. In other words, the receiving control unit (416) may compare the user's actual wake-up time with the desired wake-up time. Based on the comparison result, the environment adjustment information may be updated to change the user's circadian rhythm. Accordingly, an optimized sleep environment may be adjusted for the user, which may further increase sleep efficiency.

According to one embodiment of the present disclosure, the receiving control unit (416) collects sound information by driving the sound collection unit through a measurement mode of at least one of a manual sleep measurement and an automatic sleep measurement mode. The receiving control unit (416) may be characterized as calculating sleep state information based on the collected sound information.

In embodiments, a passive sleep measurement mode may mean that the measurement mode is initiated passively as a sleep input signal is generated by a user.

For example, the user may generate the sleep input signal by applying physical pressure to a sleep input button formed on an outer surface of the sleep environment adjustment device (400), or the user may utilize a user terminal to generate the sleep input signal. When the sleep input signal is generated, the sleep environment adjustment device (400) (i.e., the receiving module) acquires sound information related to a space as of that time. The user's sleep state information may be acquired based on that sound information. In other words, the manual spherical measurement mode allows the user to determine when to start measuring his or her sleep state.

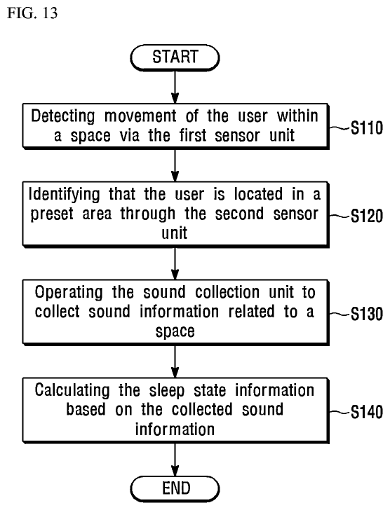

In addition, according to embodiments, the automatic sleep measurement mode may mean that sleep measurement is initiated automatically without the need for any user action to generate a sleep input signal. The automatic sleep measurement mode detects a user movement within a space through the first sensor unit. Next, when the user is identified as being located in a preset area through the second sensor unit, the automatic sleep measurement mode may be characterized by automatically initiating the measurement mode. A specific description of the automatic sleep measurement will be described below with reference to FIG. 13, and any description that is redundant of the foregoing will be omitted.

FIG. 13 is an exemplary flowchart depicting a process for generating sleep state information through an automated sleep measurement mode related to one embodiment of the present disclosure. The steps illustrated in FIG. 13 may be reordered as necessary, and at least one or more steps may be omitted or added. In other words, the foregoing steps are only one embodiment of the present disclosure, and the scope of the present invention is not limited thereto.

According to one embodiment, the receiving control unit (416) may detect movement of the user within a space (S10) via the first sensor unit.

The first sensor unit may comprise at least one of a PIR sensor and an ultrasonic sensor. The PIR sensor may detect changes in infrared light emitted by the user's body to detect movement of the user within a detection range. For example, the PIR sensor may identify infrared light emitted by the user's body in the range of 8 μm to 14 μm to detect movement of the user in the bedroom.

Ultrasonic sensors may detect object movement by generating sound waves and detecting signals that bounce off of certain objects. For example, an ultrasonic sensor may generate sound waves with in a bedroom space and detect movement of a user within the bedroom by sound waves reflecting off of the user's body as the user enter the bedroom.

According to one embodiment, the receiving control unit (416) may identify that the user is located in a preset area through the second sensor unit (S120).

### Environment Adjustment Device

According to one embodiment regarding the environment adjustment device, the receiving control unit (416) may operate the sound collection unit (414) to collect sound information related to a space (S130). In other words, the receiving control unit (416) detects that a user movement occurs in a space through the first sensor unit. And when the second sensor unit identifies a user's movement in a preset area, it may automatically cause the sound collection unit (414) to collect sound information related to a space.

### Sleep State Information

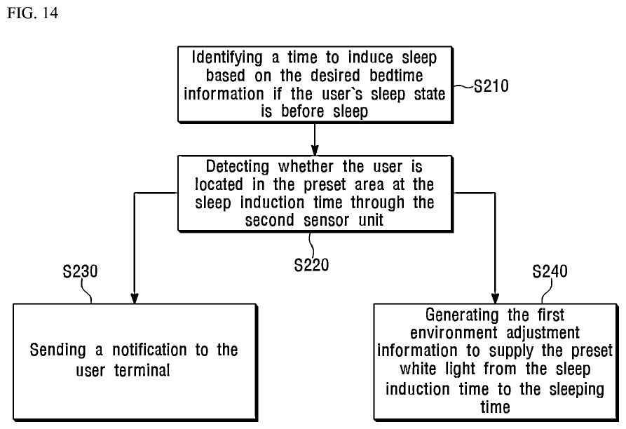

FIG. 14 is an exemplary flowchart depicting a process for adjusting an environment to induce sleep of the user related to one embodiment of the present disclosure. The steps shown in FIG. 14 may be reordered as desired, and at least one or more steps may be omitted or added. In other words, the steps described above are only one embodiment of the present disclosure, and the scope of the present invention is not limited thereto.

According to one embodiment, the receiving module (410) may identify a time to induce sleep based on the desired bedtime information if the user's sleep state is before sleep (S210). In a specific example, the user may generate sleep plan information by setting a time at which the user wishes to sleep and a time at which the user wishes to wake up through the user terminal (10), and may transmit the generated sleep plan information to the receiving module (410). In this case, the sleep plan information may include desired bedtime information and desired wake-up time information. The receiving module (410) may identify a time to induce sleep based on the desired bedtime information. For example, the receiving module (410) may determine that the sleep induction time is 20 minutes prior to when the user wants to sleep (i.e., the desired bedtime). Further examples of sleep induction time will be omitted as they are described above.

Further, the receiving module (410) may detect whether the user is located in the preset area at the sleep induction time through the second sensor unit (S220).

In an embodiment, if the receiving module (410) detects that the user is not located in the preset area, the receiving module (410), the receiving module (410) may send a notification to the user terminal (S230). Specifically, if the sleep induction time is approaching, but the user is not located in the preset area, a notification may be sent to the user terminal to prepare for sleep.

In addition, in an embodiment, when detecting that the user is located in the preset area, the receiving module (410) may generate the first environment adjustment information to supply the preset white light from the sleep induction time to the time of sleep (S240). In other words, the first environment adjustment information may be generated only when the user is located in the preset area in response to the sleep induction time.

### Environment Adjustment Information

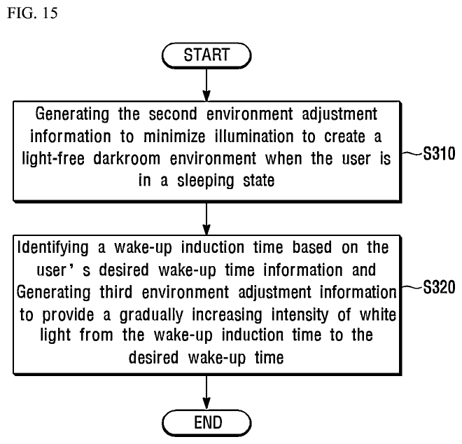

FIG. 15 is a flowchart depicting an exemplary process of changing the user's sleep environment during sleep and just before waking up related to one embodiment of the present disclosure. The steps shown in FIG. 15 may be reordered as needed, and at least one or more steps may be omitted or added. In other words, the foregoing steps are only one embodiment of the present disclosure, and the scope of the present invention is not limited thereto.

According to one embodiment, the receiving module (410) may generate the second environment adjustment information (S310) to minimize illumination to adjust a light-free darkroom environment when the user is in a sleeping state.

In other words, if the receiving module (410) detects that the user has entered sleep (or a sleep stage) (i.e., if the second sleep state information is acquired), it may generate control information to prevent light from being supplied, i.e., the second environment adjustment information. Accordingly, the probability of the user falling into a deep sleep may be increased, thereby improving the quality of sleep.

According to one embodiment, the receiving module (410) identifies a wake-up induction time based on the user's desired wake-up time information. The receiving module (410) may generate third environment adjustment information to provide a gradually increasing intensity of white light from the wake-up induction time to the desired wake-up time (S320). For example, the third environment adjustment information may be characterized as control information that causes the white light of 3000K to be supplied by gradually increasing the illuminance from 0 lux to 250 lux from the wake-up induction time to the wake-up time.

In other words, the receiving module (410) may generate the third environment adjustment information to cause the white light to be provided at a gradually increasing intensity from the wake-up induction time to the user's wake-up time, if the user's sleep state is falling asleep. For example, the third environment adjustment information related to gradually increasing the illuminance starting 30 minutes before the user's wake-up time (i.e., the wake-up induction time).

Other specific descriptions of the environment adjustment information and the operation of the smart home appliance according to the environment adjustment information are the same as those previously described through the processor (130) in FIG. 1 (a), so detailed descriptions will be omitted.

In addition, although the above describes a configuration of a sleep environment adjustment device and performing various operations accordingly, the operations described above are not limited to operations performed by the sleep environment adjustment device. For example, when the invention is practiced in an embodiment such as FIG. 1 (c), at least one of the electronic devices shown in FIG. 1 (c) may perform at least one of the operations of the sleep environment adjustment device described above.

### Configurations of Smart Home Appliances According to Some Embodiments of the Present Disclosure

### Air Conditioner

### Operations of the Air Conditioner

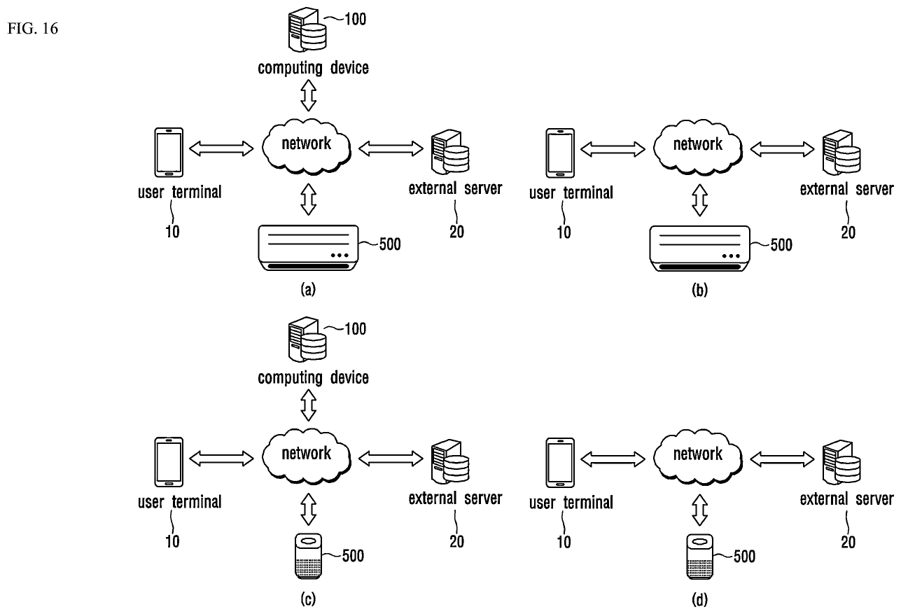

Hereinafter, an air conditioner according to the present invention will be described in detail. FIGS. 16 (a) and (b) are conceptual diagram to illustrate the operation of an air conditioner according to the present invention.

Specifically, FIG. 16 (a) is a schematic diagram in which the environment adjustment device (30) of FIG. 1 (a) is implemented as an air conditioner (500), and FIG. 16 (b) is a schematic diagram in which the air conditioner (500) operates in conjunction with the user terminal (10).

As shown in FIG. 16 (a), the air conditioner (500) according to the present disclosure may operate in conjunction with the user terminal (10) and the computing device (100).

The computing device (100) may include a network unit (110), a memory (120) and a processor (130) (see FIG. 2). The network unit (110) transmits and receives data to and from the user terminal (10), the external server (20) and the air conditioner (500). The network unit (110) may transmit and receive data or the like from other computing devices, servers, and the like for performing the sleep environment adjustment method according to the sleep state information according to one embodiment of the present disclosure. For example, the network unit (110) may receive sleep study records and electronic health records for a plurality of users from a hospital server. In another example, the network unit (110) may receive environment sensing information about a space in which a user is active from the user terminal (10). In another example, the network unit (110) may transmit environment adjustment information related to temperature and/or humidity, etc. to air conditioner (500) to adjust the environment of the space where the user is located.

The modes of operation, hardware configuration and software configuration of the network unit (110) and memory (120) are the same as described above, so redundant descriptions are omitted.

The processor (130) may read a computer program stored in the memory (120) to sleep analysis model according to one embodiment of the present disclosure. According to one embodiment of the present disclosure, the processor (130) may perform calculation to produce environment adjustment information based on the sleep state information. According to the one embodiment of the present disclosure, the processor (130) may perform calculations to train the sleep analysis model. Specific details of the sleep analysis model are the same as described above.

The processor (130) may acquire the user's sleep state information and the environment sensing information, as described above. The processor (130) may generate the first environment adjustment information or the ‘n’ environment adjustment information. Specifically, if the user's state is a pre-sleep state, the processor (130) may generate first environment adjustment information to control the air conditioner from a time when the user is predicted to be preparing to sleep (e.g., a sleep induction time) to a time when the user falls asleep (i.e., when the second sleep state information is acquired). Specifically, the first environment adjustment information may be generated to control the air conditioner to optimize the room temperature and/or room humidity to a seasonal or user-specific level by a predetermined time prior to the user's sleep (e.g., 20 minutes). Alternatively, the first environment adjustment information may include information that controls the air conditioner to generate a level of noise (white noise) that may induce sleep just prior to sleep, information that adjusts the blower intensity to a preset intensity or lower, information that lower the intensity of LED, or information that switches from direct airflow to indirect airflow. Furthermore, the first environment adjustment information may include information for controlling the air conditioner to execute dehumidification/humidification based on temperature and humidity information in the sleeping space.

Based on the second sleep condition information, the processor (130) may generate second environment adjustment information to reduce the brightness of the display of the air conditioner, turn off the display, operate the air conditioner at a noise level below a preset level, adjust the blowing intensity to a preset intensity, adjust the blowing temperature to a predetermined range, maintain the humidity in the sleeping space at a predetermined value or maintain an indirect airflow to control the air conditioner.

Further, the processor (130) may generate the third environment adjustment information and the fourth environment adjustment information based on the third sleep condition information and the fourth sleep condition information.

The processor (130) may determine to transmit the environment adjustment information to the air conditioner (500). In other words, the processor (130) may improve the quality of the user's sleep by generating external environment adjustment information that may facilitate the user falling asleep or waking up naturally.

As shown in FIG. 16 (b), the air conditioner (500) according to the present disclosure may operate in conjunction with the user terminal (10). In other words, the system of the embodiment according to FIG. 16 (b) may include the air conditioner (500), the user terminal (10), the external server (20), and the network. In this embodiment, the air conditioner (500) according to the present disclosure includes the configuration of the computing device (100) of FIG. 16 (a) and additional components for operating as an air conditioner.

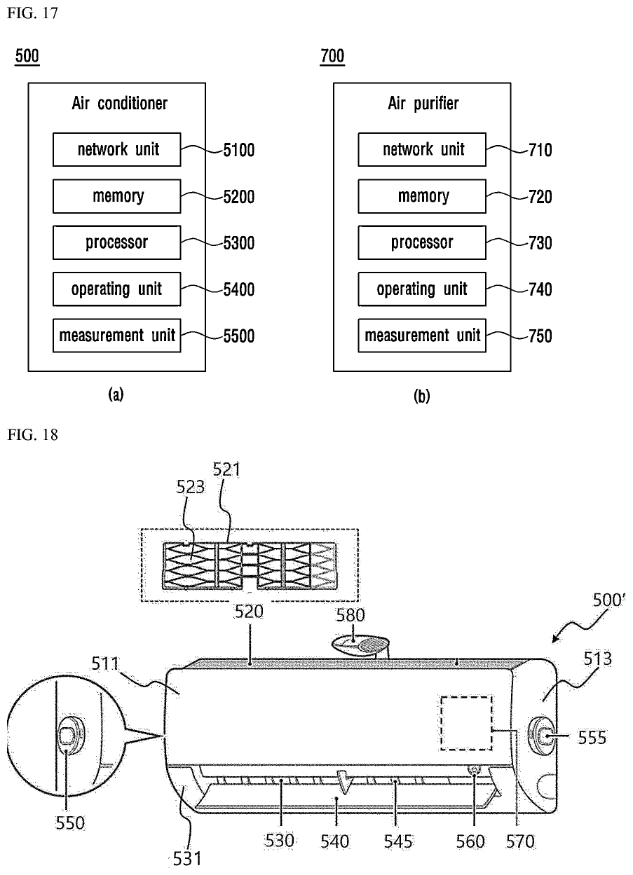

FIG. 17 (a) is a block diagram depicting a configuration of an air conditioner according to the present invention. As shown in FIG. 17 (a), an air conditioner (500) according to the present disclosure may include a network unit (5100), a memory (5200), a processor (5300), an operating unit (5400), and a measurement unit (5500).

The air conditioner (500) may be implemented as a wall-mounted air conditioner or a wall-mounted heating and cooling air conditioner that is fixedly installed on a wall within a building, apartment, or house. The air conditioner (500) may also be implemented as a ceiling-mounted system air conditioner. The air conditioner (500) may be implemented as a stand air conditioner that is placed on one side or in a corner of an interior space. The air conditioner (500) may also be implemented as a portable air conditioner that is easy to carry and move around.

The function, operation, hardware configuration, and software configuration of the network unit (5100), memory (5200), and processor (5300) of the air conditioner (500) are described above. The first to n-th environment adjustment information generated by the processor (5300) may be transmitted to the operating unit (5400). The operating unit (5400) operates various hardware elements of the air conditioner (500).

The measurement unit (5500) may include one or more sensors for sensing temperature, humidity, dust concentration and air conditioner component status within the sleeping space. Specifically, the measurement unit may include a dust sensor to detect invisible suspended particles such as PM 1.0, PM 2.5, PM 10, etc., an illuminance sensor to detect room illumination, a temperature sensor to measure room temperature, a humidity sensor to measure room humidity, etc.

In addition, the measurement unit (5500) may include a human body detection sensor. The processor (5300) may detect a user through the human body detection sensor and perform control to send cool or warm air to a space where the user is located (direct air) or to send cool or warm air to a space where the user is not located (indirect air).

In addition, the measurement unit (5500) may further include a voice recognition sensor to recognize the user's voice.

Although not shown, the air conditioner (500) may comprise a housing, a filter, a blower fan, a blower fan, a sterilization unit, a humidification unit, a heating unit, a cooling unit and a measurement unit with an outlet and an inlet. The housing may be designed in various ways depending on the implementation of the air conditioner (500), such as a wall-mounted air conditioner, a system air conditioner, a stand air conditioner, etc. The filter unit may be selected according to a dust collection filter type, an adsorption filter type etc. The blower fan may be connected to a motor rotated by the power supplied from the power supply. The sterilization unit has the function of sterilizing the inhaled air using chemical and electrical methods. The humidifying unit has the function of humidifying and transmitting the inhaled air, and the heating and cooling unit has the function of heating or cooling the inhaled air to a predetermined temperature.

The hardware elements of the air conditioner (500) described above are only one embodiment, some of which may be integrated and implemented in one configuration, and some of which may be omitted. Various configurations may be added to perform to perform air purification functions not described above.

On the one hand, the environment sensing information may be acquired through the user terminal (10). The environment sensing information may be sleep sound information acquired in a bedroom where the user sleeps.

The environment sensing information may also be temperature and/or humidity or air quality information in the sleeping space acquired from the measurement unit (5500) provided within the air conditioner (500). The environment sensing information acquired through the user terminal (10) or the measurement unit (5500) may be the basis for acquiring the user's sleep state information in the present invention.

As a specific example, the environment sensing information acquired related to a user's activity may be used to acquire sleep state information regarding whether the user is before sleep, during sleep, or after sleep. In addition, information regarding ambient air quality before, during and after the user's sleep may be acquired.

The processor (5300) may acquire the sleep state information based on the environment sensing information acquired through the user terminal (10) and/or the measurement unit (5500).

Specifically, the processor (5300) may identify singularities in the environment sensing information where information of a predetermined pattern is detected. Here, the information of a preset pattern may be related to breathing and movement patterns related to sleep. For example, in the wake state, breathing patterns may be irregular and body movements may be high because all nervous systems are active. Also, the throat muscles are not relaxed, so breathing sounds may be very low. On the other hand, when the user is sleeping, the autonomic nervous system is stabilized, and the breathing pattern may become more regular, the body movements may become less active, and the breathing sound may become louder. In other words, the processor (5300) may identify as a singularity a point when a predetermined pattern of sound information regarding regular breathing, less body movement, or less breathing sound is detected in the environment sensing information. Further, the processor (5300) may acquire sleep sound information based on the environment sensing information acquired based on the identified singularity. The processor (5300) may identify a singularity in the time-series acquired environment sensing information regarding the user's sleeping time, and may acquire the sleep sound information based on the identified singularity.

Furthermore, temperature and/or humidity and/or air quality measured by the measurement unit (5500) has a significant impact on the user's sleep. Paper analyzing the relationship between temperature and/or humidity and sleep shows differences in the frequency of awakening during sleep and the percentage of deep sleep, adversely affecting the user's work efficiency of the next day. According to paper analyzing the relationship between air quality and sleep, sleep disruption is statistically significantly related to air pollution. For example, in an experiment comparing CO2 (800 rpm, 1700 rpm) and temperature (24 degrees, 28 degrees), it was found that sleeping in a 28-degree chamber decreased sleep efficiency and work efficiency the next day, while at 880 ppm CO2, the air felt more stuffy and hotter. Furthermore, in an experiment comparing sleep conditions at 32° C. (80% relative humidity) and 26° C. (50% relative humidity), it was found that the frequency of awakenings during sleep at 32° C. (80% relative humidity) increased and the percentage of deep sleep decreased. On the one hand, exposure to PM 10 may cause difficulty in maintaining sleep, especially, it was confirmed that the probability of generating sleep disorder is highest when men are exposed to PM 1. In addition, it was confirmed that women are most likely to have sleep disorders when exposed to PM 1 and PM 2.5. Also, it was confirmed that the probability of sleep disturbance related to wheezing was highest when SO2 and O3 were high. In addition, it was confirmed that when pregnant women were exposed to PM 2.5 between 31 and 35 weeks of gestation, their children were most likely to have shorter sleep length. Various studies have been conducted on the relationship between the AHI and air quality measures, and the results are slightly different for each study, but the results are the same that the correlation between air quality and sleep is very high.

The air conditioner (500) according to the present disclosure may acquire sleep state information based on the environment sensing information, generate the environment adjustment information, and perform an operation appropriate for a sleep stage by using the generated environment adjustment information.

Specifically, the processor (5300) of the air conditioner (500) may generate first environment adjustment information for controlling the air conditioner from a time when the user is predicted to be preparing for sleep (e.g., a sleep induction time) to a time when the user falls asleep (i.e., when the second sleep state information is acquired) if the user's condition is determined to be a pre-sleep state. The first environment adjustment information may be generated by reflecting PM concentration, harmful gas concentration, CO2 concentration, SO2 concentration, O3 concentration, humidity, temperature, etc. measured at the measurement unit (5500).

The first environment adjustment information may include information for controlling the air conditioner to optimize the room temperature and/or room humidity until a predetermined amount of time (e.g., 20 minutes) prior to the user's sleep, information for controlling the air conditioner, adjusting the blower intensity below a preset intensity, lowering the intensity of the LED, or converting direct air to indirect air for generating noise (white noise) at a level that may induce sleep just prior to sleep, information that reduce the, information that switch from direct airflow to indirect airflow, information for controlling the air conditioner to execute dehumidification/humidification based on the temperature and humidity information within the sleeping space, and the like.

In addition, the processor (5300) may generate a second environment adjustment information to reduce the brightness of the display of the air conditioner, turn off the display, operate the air conditioner at a noise level below a predetermined level, adjust the blowing intensity below a preset intensity, adjust the blowing temperature to a preset range, maintain the humidity in the sleeping space at a predetermined temperature, or maintain indirect airflow based on the second sleep adjustment information.

The second environment adjustment information is based on the second sleep state information, and may be control information for controlling the air conditioner to reduce the brightness of the display unit of the air conditioner, turn off the display unit, operate the air conditioner with a noise below the preset level, adjust the blowing intensity below a preset intensity, adjust the blowing temperature to a preset range, maintain the humidity in the sleeping space at a predetermined temperature, or maintain an indirect airflow. The user may be induced to sleep by airflow, white noise etc. in a sleeping space having an optimized temperature and humidity just before sleeping, and may able to have a good night's sleep with an optimized temperature and humidity just before sleep, and may able to have a good night's sleep with an optimized temperature, humidity etc. in the controlled state after sleeping.

### Configuration of the Air Conditioner According to an Embodiment

FIG. 18 is a diagram to illustrate one example of the air conditioner shown in FIG. 16 and FIG. 17.

Referring to FIG. 18, an air conditioner according to one example of the present invention includes an indoor unit (500′). In one example, the indoor unit (500′) may be a wall-mounted indoor unit that is mounted on a wall of an indoor space.

The indoor unit (500′) includes a casing (511, 513) forming an exterior. The casing (511, 513) includes a front unit (511) forming the front appearance of the indoor unit, and side unit (513) provided on both sides of the front portion (511) and extending rearward from the front unit (511) toward a wall.

And the casing (511, 513) further includes a rear unit (not shown) disposed at the rear of the side unit (513). The rear unit (not shown) is disposed between the two side units (513) and may be coupled to wall of the interior space.

The front unit (511), the two side units (513), and the rear unit (not shown) form an interior space, which may accommodate a plurality of components provided in the indoor unit (500′). The plurality of components may include a heat exchanger (not shown), a fan (not shown), a processor (not shown), etc.

The indoor unit (500′) further includes a filter assembly (520) disposed on top of the casing. The filter assembly (520) may form a top surface of the casing. And, the filter assembly (520) is formed with a plurality of filter inlets (523) that draw air from the indoor space into the interior of the indoor unit (500′). The air drawn from the filter inlets (523) may be cooled or heated as it passes through the heat exchanger.

The filter assembly (520) may be disposed in an inlet formed on a top surface of the casing through which air is drawn to filter the air. The inlet may be an opening formed by the upper surface of the front unit (511), both side units (513), and rear unit (not shown) of the casing.

The filter assembly (520) may include a filter member (521). The filter member (521) may be a filter for filtering out dust, ultra-fine dust, or the like, and may be an antimicrobial micro filter with an antimicrobial function.

The filter member (521) may be configured with a plurality of filter inlets (523). In this case, the filter member (521) may comprise a plurality of frames for forming the plurality of filter inlets (523). Furthermore, the filter inlets (523) may be arranged with a net to filter the inhaled air.

The indoor unit (500′) further includes an outlet panel (531), disposed at a lower part of the casing and having an outlet (530) through which air inhaled into the indoor unit (500′) is output. The outlet (530) may be equipped with an up-down wind regulator (540), which is movably provided to regulate the output direction or air volume of air output from the outlet (530). For example, the up-down wind regulator (540) may be provided to rotate back and forth centered on a hinge axis provided at both ends of the wind regulator (540) to adjust the direction of the wind up or down. In addition, the outlet (530) may be equipped with a left or right wind directional adjuster (545), which is movably provided to adjust the output direction or air volume of air output form the outlet (530).

When the fan is operated, air from the indoor space is drawn into the interior of the indoor unit (500′) through the filter assembly (520) and exchanged heat in the heat exchanger. Then, the heat exchanged air may be exchanged air may be output through the outlet (530).

In this embodiment, it has been described that the filter assembly (520), which draws air into the indoor unit, is located at the top of the indoor unit, and the outlet (530), which outputs air, is located at the bottom of the indoor unit (500′). However, conversely, the filter assembly (520) may be located at a lower portion of the indoor unit and the outlet (530) may be located at an upper portion of the indoor unit. As another example, an additional filter assembly or outlet may be formed in the front unit (511) of the casing.

In summary, the indoor unit (500′) according to the present invention may be configured to generate airflow such as upper intake and lower output, lower intake and upper output, front intake and lower output, upper intake and front output, and the like.

The side unit (513) of the casing may be equipped with a sensor device (550) capable of detecting the amount of dust included in the air of the indoor space. By installing the sensor device (550) on the side portion (513), the amount of dust may be detected by drawing only a small amount of air into the sensor device (550), without being affected by the intake and output airflow through the indoor unit (500′).

On the side unit (513) of the casing, a voice recognition sensor (555) may be disposed to recognize the user's voice.

On the front unit (511) of the casing, a force operation button (560) may be disposed, which may be used to force the air conditioner to turn on or off, or to perform a test run.

On the front unit (511) of the casing, a display unit (570) may be disposed that allows the user to check the desired temperature and the operation status of the additional functions when the air conditioner is operated. Here, the display unit (570) may be disposed with a sensor that receives a remote control signal.

The ion generator (580) is disposed on a top side of the indoor unit (500′), and is a means for diffusing ions to both sides. More specifically, the ion generator (580) is coupled to a frame inside the casing, and is a means for diffusing ions to both sides.

By generating high voltage to both sides, the ion generator (580) may ionize molecules in the air, whereby dust is charged by the ionized molecules, and the charged dust may be effectively collected by the filter assembly (520).

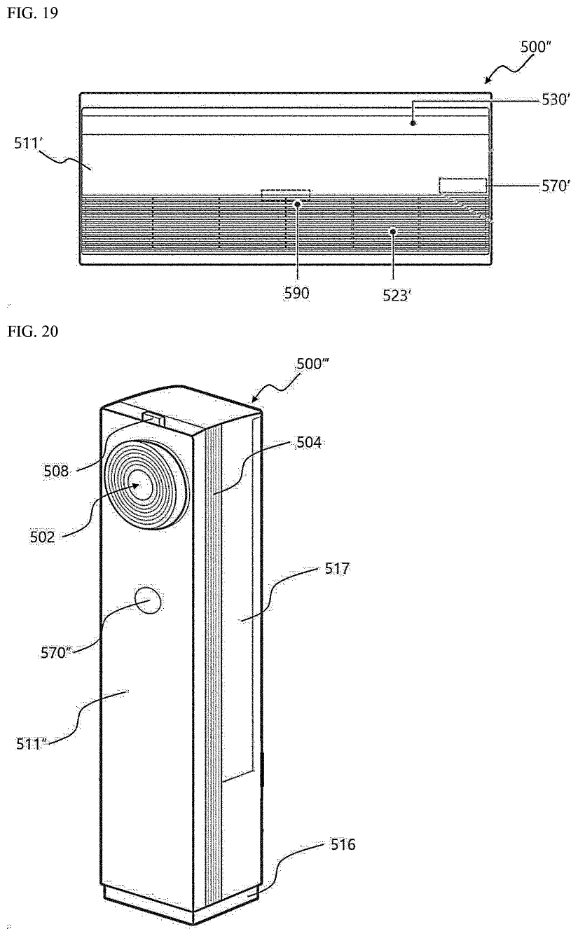

FIG. 19 is a diagram to illustrate another example of the air conditioner shown in FIG. 16 and FIG. 17.

Referring to FIG. 19, the air conditioner according to another example of the present disclosure includes the indoor unit (500″). In one example, the indoor unit (500″) may be an indoor unit for a system air conditioner that is installed in the ceiling of an indoor space.

According to another example of the present invention, the indoor unit (500″) includes a casing. The casing may include the front unit (511′). Although not shown in the diagrams, the casing may further include other units other than the front unit (511′). The front unit (511′) may be the unit that is visible when the user is looking at the ceiling.

The front unit (511′) may be formed with an inlet (523′) through which room air is drawn in and an outlet (530′) which cold or hot air is output.

The display unit (570′) may be disposed on the front unit (511′), wherein the operating status of the air conditioner may be checked. The receiving unit may be disposed in the display unit (570′) that receives a signal from a wireless remote control. Also, the force operation button may be disposed.

On the front unit (511′), an air quality indicator light (590) may be disposed, which displays various colors depending on the indoor air condition.

A predetermined interior space is formed inside the casing, and the interior space may accommodate a plurality of components provided in the indoor unit (500″). The plurality of components may include a heat exchanger (not shown), a fan (not shown), a processor (not shown), and the like.

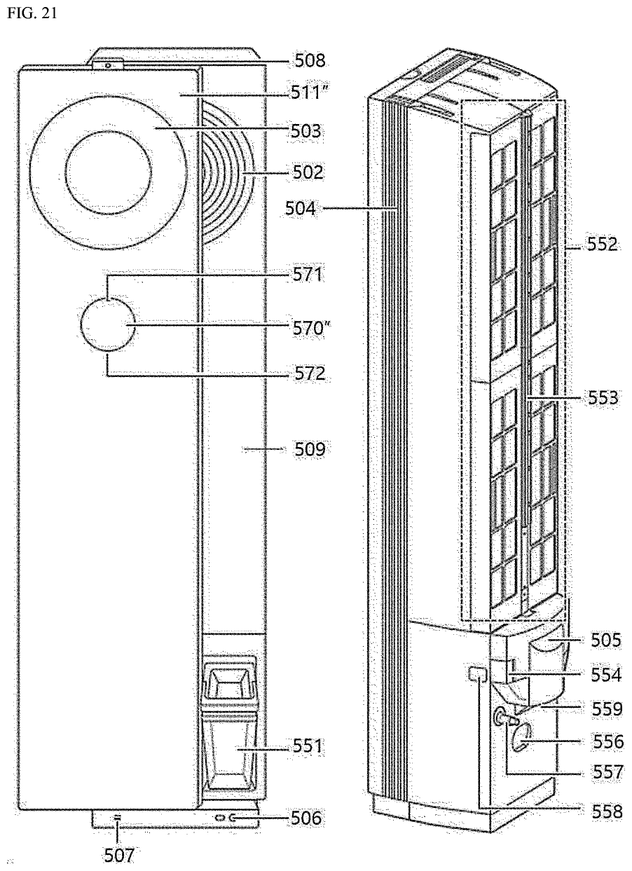

FIG. 20 and FIG. 21 are diagrams to illustrate another example of the air handler shown in FIG. 16 and FIG. 17.

Referring to FIG. 20 and FIG. 21, an air conditioner according to another example of the present invention includes an indoor unit (500′″). In one example, the indoor unit (500′″) may be a stand indoor unit that is installed on the floor of an indoor space.

The indoor unit (500″″) may be provided indoors and connected to an outdoor unit (not shown) disposed outdoors via refrigerant pipe (not shown).

The indoor unit (500′″) may include the front unit (511″) forming a front exterior and a circulator door (503) disposed on the front unit (511″) and movable to open and close in an upward and downward direction.

The indoor unit (500″″) may include a base (516), a cabinet (517), and a front unit (511″). The front unit (511″) forms the front exterior of the indoor unit (500″″), and the cabinet (517) may be installed to be positioned on top of the base (516).

The circulator door (503) may be installed on the front unit (511″).

The indoor unit (500″″) may include an air inlet and an air outlet, and may condition the air inhaled through the air inlet and output it through the air outlet. For example, the air inlet may be formed at the rear of the indoor unit (500″″) and the air outlet may be formed at the top of the front of the indoor unit (500″″). Alternatively, the air inlet may be disposed on the outdoor unit. Here, the inlet and outlet may be formed at different locations on the indoor unit (500′″). For example, the outlet may be formed on a side of the bottom of the indoor unit (500″). It may also be possible for multiple outlets to be formed at the top of the front of the indoor unit (500′″), and at the sides of the bottom of the indoor unit (500″″). The inlets may be formed at one or more of a rear of the main body, a front of the lower portion, and a side. A filter unit (not shown) may be installed at the inlet to filter out foreign matter such as dust included in the inhaled air.

A cleaning module (505) for cleaning the moving filter (552) may be disposed in the indoor unit (500″).

At the rear of the closed circulator door (503), a circulator module (502) may be provided. The circulator module (502) may generate a blowing force to draw in air through the inlet and output air through the outlet.

The circulator module (502) may be installed within the interior of the indoor unit (500″) and, in operation, may output air through the outlet exposed by the opening of the circulator door (503).

The circulator module (502) may be operated by forward movement of the circulator door (503) to an open outlet. For example, after at least a portion of the circulator module (502) is moved forward to pass through the circular outlet where the circulator door (503) moves downward to open, the circulator fan of the circulator module (502) may rotate and operate.

As described above, as user herein, the output may refer to an opening through which at least a portion of the circulator module (502), the output unit for outputting air, passes.

A circulator door (503) may open and close the outlet. The circulator door (503) may open and close the main outlet and may be configured to output treated air from the air conditioner, such as heat exchanged air, purified air, etc. to the outside.

The circulator door (503) opens during main body operation to expose the circulator module (502) to the outside to output air through the outlet, and closes to close outlet when operation ends. An interior or rear side of the front unit (511″) may be provided with a space to accommodate the circulator door (503) when the outlet is open.

A movement means (not shown) for moving the circulator door (503) may be installed in an inner side of the front unit (511″). For example, the inner side of the front unit (511″) may include a circulator door motor, a gear member, a rail member, or the like for moving the circulator door (503) in an upward or downward direction based on rotation of the circulator door motor. On the one hand, a step motor, which has a low cost and is easy to control, may be used as the circulator door motor. In this case, the circulator door motor may be named a circulator door step motor.

The circulator door (503) may be configured to open from an interior side of the indoor unit (500′″) by moving in an upward or downward direction. Since the circulator door (503) is disposed on an upper side of the front unit (511″) of the indoor unit (500′″), it is more preferable from a space utilization perspective that the circulator door (503) is configured to move in a downward direction to open.

Alternatively, the circulator door (503) may be configured to open by moving in an upward or a downward direction after an inwardly backward movement of the indoor unit (500″). In this case, the circulator door (503) may be configured to open by moving in a downward direction after the indoor unit (500″″) is moved backward inwardly, which is more preferable in terms of space utilization.

Hereinafter, an example in which the circulator (503) is moved in the upward direction to open and close will be described. The circulator door (503) may be opened by retracting in an inward direction and then moving in a downward, and the circulator door (503) may be closed by moving in an upward direction and then advancing in a frontward direction.

When the circulator door (503) is opened, the circulator module (502) may move forward in a forward direction toward the front unit (511″) to output air. In addition, upon termination of the operation, the circulator module (502) may move backwardly toward the interior of the indoor unit (500″″) and close the outlet by movement of the circulator door (13).

In some cases, a blower fan (not shown) may be further installed in the interior of the main body to assist in blowing.

In addition to the circulator module (502), the interior of the indoor unit (500′″) may further include a plurality of blower fans. For example, the plurality of blower may be disposed on the underside of the circulator module (502).

Meanwhile, an auxiliary outlet (504) may be further installed on the side of the cabinet (517). Furthermore, a wind control means for adjusting the wind direction of the output air may be disposed at the auxiliary outlet (504).

By providing the circulator module (502) on the top of the indoor unit (500′″), it is easier to send wind to a distance.

Also, by positioning the circulator module (502) at the final stage of the air output path, the heat-exchanged air and the purified air may be output directly to a distance.

After the circulator door (503) is opened, the circulator module (502) which is the output unit may be configured to rotate in two dimensions. For example, the circulator module (502) may include a rotation unit comprising a two-axis rotation structure utilizing a double joint, gear rack structure, so that the circulator module may rotate freely in various directions. Accordingly, the circulator module (502) may be rotated as desired by the user to control the airflow.

After the entire circulator module (503) is rotated, the wind is sent to the user's desired location for intensive cooling, which further enhances the user's comfort and satisfaction.

A display unit (570″) may be disposed on the front unit (511″). The display unit (570″) may display operation state and setting information of the indoor unit (500″″), and may be configured as a touch screen to receive user commands. According to an embodiment, the front unit (511″) may be equipped with a control unit (not shown) that includes at least one input means of a switch, button, or touchpad.

One side of the display unit (570″) may be equipped with a proximity sensor (571) and a remote control receiver (572). According to an embodiment, when a proximity signal corresponding to a user's approach is input from the proximity sensor (571), the display unit (570″) may be activated may be activated to display operation information, and the at least one light provided in the indoor unit (500′″) may be operated.

The display unit (570″) may further include one or more lights.

An automatic door opening sensor (506) may be installed on the base (516). The automatic door opening sensor (506) may detect a user approaching the indoor unit (500″″), and may cause the front unit (511″) to open or close. On the one hand, the automatic door opening sensor (506) may be disposed in a lower predetermined area of the front unit (511″).

A microphone and/or speaker (507) may be disposed on the base (516). The microphone and/or speaker (507) may recognize the user's voice and communicate information to the user by voice.

The indoor unit (500″″) may further include a human body detection sensor (508). The human body detection sensor (508) may be disposed on an upper portion of the front unit (511″). The human body detection sensor (508) may detect a person and control the wind to flow in the direction of a person or in the direction of no person depending on the driving mode. On the one hand, a vision module including at least one camera may be installed on the upper portion of the front unit (511″).

The indoor unit (500′″) may include, inside, a heat exchanger (not shown) for heat exchange for heat exchange of the inhaled air with a refrigerant.

The front unit (511″) may be moved by sliding to the left or right. Thus, the front unit (511″) may also be named a sliding door.

The front unit (511″) is mounted by sliding means formed on the cabinet (517) and may be moved left and right. Movement of the front unit (511″) may expose a portion of the interior panel (509) to the outside.

The cabinet (517) may include a sliding door step motor, a gear member for moving the front unit (511″) in a left or right direction based on rotation of the sliding door step motor, and a rail member etc.

The interior panel (509) may accommodate the circulator module (502) and may be equipped with moving means (not shown) for moving the circulator module (520).

In some embodiments, the circulator module (502) may include a circulator fan (not shown), a circulator rotation unit (not shown) that may be rotated to change the direction in which at least the circulator fan (now shown) is facing, and at least a circulator movement unit (not shown) that may move the circulator fan (not shown).

The inner panel (509) may have a water container (551) installed at the bottom of the humidification module. The water container (551) may be exposed to the outside as the front unit (511″) moves in a left or right direction to open.

A predetermined area of the humidifying water container (551) may be formed with an inlet for filling the water. In some embodiments, the inlet may be open, or a cover may be disposed to open or close at least a portion of the inlet.

The water container (551) may be connected to the cabinet (517) with a movable shaft formed at its lower end. An upper unit of the water container (551) may be formed to protrude forwardly, relative to the lower shaft of movement, to open an inlet. The water container (551) may be tilted forward such that the top of the reservoir (551) forms a small angle with the interior panel (509).

In addition, the water container (551) may be detachable from the indoor unit (500″″). An interior of the indoor unit (500″″) may be provided with a sensor to detect whether the water reservoir (551) is mounted.

When the user's approach is detected by the proximity sensor (517), the water container may automatically move in response to the proximity signal to open the spout. The water container (551) may move to open the inlet as the handle (not shown) is pulled toward the front. The water container (551) may move toward the front to open the inlet as the fastening (not shown) is released by being pressed inwardly. The water container (551) may automatically rotate to open the inlet as the front unit (511″) slides open.

The interior panel (509) or a portion of the water container (551) may be provided with a water level indicator (not shown) to display the water level in the water container (551).

The water container (551) may be configured to allow the amount of water inside to be checked. For example, the water container (551) may be formed of a material with transparent front surface. The water reservoir may be formed of a material such that a portion of the front is transparent. Additionally, the water container (551) may be formed entirely of a transparent material.

The indoor unit (500′″) includes a filter (552). The filter (552) may be disposed at the rear of the indoor unit (500′″).

The indoor unit (500′″) includes a room temperature sensor (553). The room temperature sensor (553) senses the room temperature and may be disposed at the rear of the indoor unit (500′″).

The indoor unit (500′″) may include a dust bin (554) for storing dust collected by the cleaning module (505).

The indoor unit (500′″) may further include a humidity sensor (550). The humidity sensor (559) senses indoor humidity and may be disposed at a rear of the indoor unit (500′″).

The indoor unit (500″″) may further include a plumbing hole (556) and a drain hole (557). The plumbing hole (556) and the drain hole (557) may be disposed at the rear of the indoor unit (500′″).

The indoor unit (500′″) may include a PM 1.0 sensor (558). The PM 1.0 sensor (558) senses fine particulate matter and may be disposed at the rear of the indoor unit (500′″).

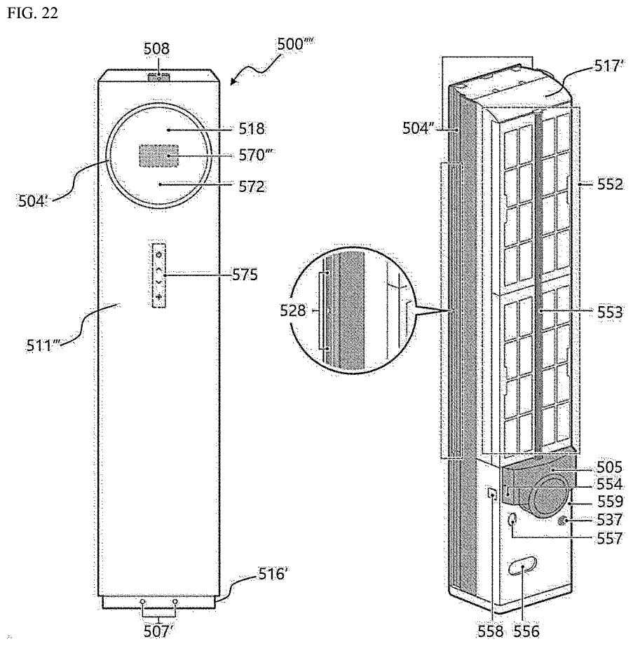

FIG. 22 is a diagram to illustrate another example of the air conditioner shown in FIG. 16 and FIG. 17.

Referring to FIG. 22, an air conditioner according to another example of the present invention includes an indoor unit (500″″). For example, the indoor unit (500″′″) may be a stand indoor unit that is installed on the floor of an indoor space.

The indoor unit (500″″) includes a front unit (511′″). The front unit (511′″) forms the front exterior of the indoor unit (500″″).

The indoor unit (500″″) may include a circle unit (518). The circle unit (518) may be disposed in a circular opening formed in the front unit (511′″).

The indoor unit (500″″) may include a display unit (570′″). The display unit (570′″) may be disposed on the front unit (511′″). The display unit (570″″) may be disposed on the circle unit (518). The display unit (570″″) may display operation state and setting information. Here, the display unit (570″″) may be configured as a touch screen to receive user commands.

The indoor unit (500″″) may include a remote control receiver (572). The remote control receiver (572) may be disposed on the circle unit (518) and may be disposed on one side of the display unit (570′″).

The indoor unit (500″″) may include an indoor unit button portion (575). The indoor unit button portion (575) may be disposed on the front portion (511″″). The indoor unit button portion (575) may turn the power on and off, or set the temperature and wind intensity, without the need for a remote control.

The indoor unit (500″″) may have outlets (504, 504″) for output air.

The first outlet (504) may be formed on the front unit (511″″) and may have a ring shape surrounding the circle unit (518). The first outlet (504) may be an opening between the front unit (511′″) and the circle unit (518). The second outlet (504″) may be a left and right outlet of the indoor unit (500″″), which may send air into the room from either side to adjust the room temperature to a user-desired or preset temperature.

The indoor unit (500″″) may include an air guard (528). The air guard (528) may be disposed on either side of the indoor unit (500″″) and may adjust the direction of the air output from the second outlet (504″).

The indoor unit (500″″) may include a microphone (507′). The microphone (507′) may be disposed on a base (516′) that comprises a bottom unit of the indoor unit (500″″).

The indoor unit (500″′″) may include a speaker (537). The speaker (537) may be disposed in a cabinet (517) of the indoor unit (500′″).

The indoor unit (500″″) may include a room temperature sensor (553). The room temperature sensor (553) senses the room temperature and may be disposed at the rear of the indoor unit (500″″).

The indoor unit (500″″) may include a dust bin (554) for storing dust collected by the cleaning module (505).

The indoor unit (500″″) may further include a humidity sensor (559). The humidity sensor (559) senses indoor humidity and may be disposed at a rear of the indoor unit (500″″).

The indoor unit (500″″) may further include a plumbing hole (556) and a drain hole (557). The plumbing hole (556) and the drain hole (557) may be disposed at the rear of the indoor unit (500′″).

The indoor unit (500″″) may include a PM 1.0 sensor (558). The PM 1.0 sensor (558) senses fine particulate matter and may be disposed at the rear of the indoor unit (500″″).

The indoor units (500′, 500″, 500″, 500″″) shown in FIGS. 18 to 22 have display units (570, 570, 570″, 570′″).

Since the indoor units (500′, 500″) shown in FIG. 18 and FIG. 19 are installed on the top of a wall or on a ceiling, it is not easy for a user to control the indoor units (500, 500″) through the display units (570, 570′). Therefore, their display units (570, 570′) are used to display that indoor units (500′, 500″) are controlled by a user through a remote control or a user terminal, or to display the current status of the indoor units (500′, 500″). The display unit (570″″) of the indoor unit (500″″) shown in FIG. 22 is also used to display that the indoor unit (500″′″) is controlled by a user through a remote control or a user terminal, or to display the current status of the indoor unit (500″″).

On the one hand, the display unit (570″) shown in FIG. 20 and FIG. 21, unlike the other display units (570, 570′, 570″), is configured as a touch screen so that user commands may be directly input, and a display screen may be displayed according to the input commands. Also, the display unit (570″), like the other display units (570, 570′, 570″), may be used for displaying that the indoor unit (500″″) is controlled by a user through a remote control or a user terminal, or for displaying the current status of the indoor unit (500′″).

As described above, the indoor units (500′, 500″, 500′″, 500′″) shown in FIGS. 18 to 22 may be controlled for power, operation mode, and other settings through a remote control, a user terminal, or a display. Control signals input through the remote control, user terminal, or display may be input to the processor (5300) shown in FIG. 17, and the processor (5300) may control the operating unit (5400) based on the input control signals.

The operation mode of the indoor units (500′, 500″, 500′″, 500″″) may include various operation modes. For example, it may include a cooling mode, an automatic mode, a dehumidifying mode, a heating mode, a blowing mode, an air cleaning mode, a power saving mode, etc.

An air conditioner including an indoor unit (500′, 500″, 500′″, 500″′″) shown in FIGS. 18 to 22 may further include a ‘sleep mode’ as an operation mode. The sleep mode is a mode in which the temperature and/or humidity of the indoor space is controlled to improve the quality of sleep for a user.

Based on the first environment adjustment information or the n-environment adjustment information, or the A environment adjustment information or the H environment adjustment information generated by the computing device (100) of FIG. 16 (a) or the air conditioner (500) of FIG. 16 (b), the air conditioner (500) may optimize the temperature and/or humidity, etc. in the sleeping space for sleep.

For example, the air conditioner (500) may adjust the room temperature and/or room humidity for entering sleep by a predetermined time (e.g., 20 minutes) based on the first environment adjustment information. Alternatively, the air conditioner (500) may switch from direct airflow to indirect airflow, may generate noise (white noise) at a level that may induce sleep just prior to sleep, may adjust the blowing intensity to a preset intensity or less, or may reduce the brightness of the display units (570, 570′, 570″, 570′″) shown in FIGS. 18 through 22.

The air conditioner (500) may further reduce the brightness of the display units (570, 570′, 570″, 570′″), turn off the display units (570, 570′, 570″, 570″″) according to second environment adjustment information, operate at a noise level below a preset level, adjust the blowing intensity below a preset intensity, maintain a room temperature within a preset range, maintain humidity within the sleeping space at a predetermined temperature, or maintain indirect airflow. The air conditioner (500) may adjust the room temperature and/or room humidity for a wake up, lower the blowing intensity and noise during a wake-up time, generate white noise to gradually induce a wake-up, keep the noise below a preset level, or operate in conjunction with a wake prediction time or weather recommendation time according to the third environment adjustment information. The air conditioner (500) may control at least one of room temperature, room humidity, blowing intensity, and noise level according to the fourth environment adjustment information, and may acquire, analyze, and reflect user data.

For example, the air conditioner (500) may be turned on based on the A environment configuration information, the air volume may be changed to a certain intensity or less based on the B environment configuration information, the current air volume may be lowered to a certain intensity or less, direct air may be switched to indirect air or no air, or the brightness of the display unit may be may be lowered to a predetermined brightness or less.

The set temperature and humidity based on the C environment adjustment information may be changed to an optimal temperature and humidity. The humidity or temperature set based on the D environment adjustment information may be increased, or the direct wind or indirect wind may be switched to no wind. The temperature or humidity of the bedroom may be changed to an optimized temperature or humidity based on the E environment configuration information. The set temperature and humidity may be changed to a preferred temperature or humidity that the user has typically set upon entering sleep in the past. The set temperature or humidity may be changed to a specific temperature or humidity that is most preferred by the user based on the G environment adjustment information. The set temperature or humidity may be changed to a specific temperature or humidity that is most preferred by the user based on the H environment adjustment information.

In the case of the system configuration of FIG. 16 (a), in order for the air conditioner (500) to operate in a sleep mode, a device connection process may be performed for the air conditioner (500) to be connected through a network with the computing device (100).

In the case of the system configuration of FIG. 16 (b), in order for the air conditioner (500) to operate in sleep mode, the air conditioner (500) may perform a connection process for linking with the user terminal (10). Here, the air conditioner (500) and the user terminal (10) may be wirelessly connected. For example, the air conditioner (500) and the user terminal (10) may be directly connected through a local area network.

### Method for Sleep Mode Operation of an Air Conditioner

Operating an air conditioner including the indoor unit (500′, 500″, 500′″, 500″″) shown in FIGS. 18 through 22 into a sleep mode may have method by operating the indoor unit (500′, 500″, 500″, 500″″) into a sleep mode at the user's option, and method by automatically operating the sleep mode.

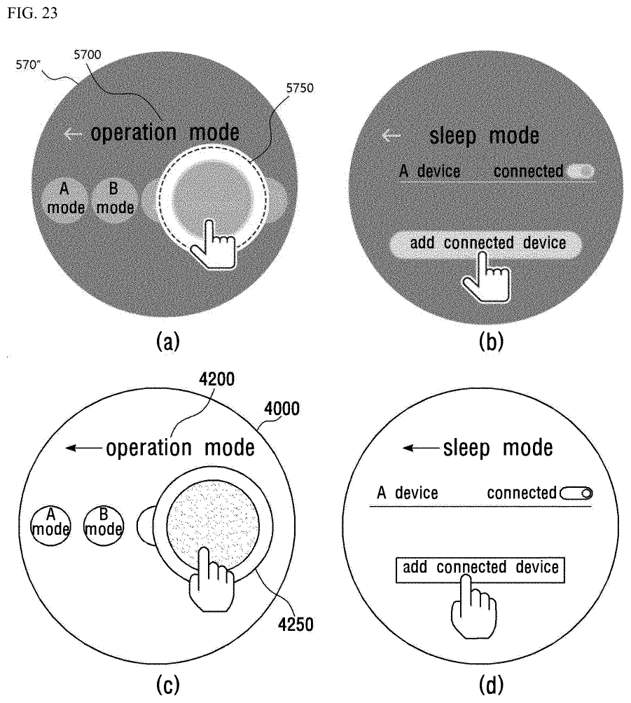

First, a method of operating the indoor unit (500′, 500″, 500″, 500″′″) into a sleep mode according to a user's selection will be described with reference to FIGS. 23 to 25.

FIGS. 23 (a) and (b) are diagrams to illustrate how to operate the indoor unit (500″) into a sleep mode through the display unit (570″) of the indoor unit (500″) shown in FIGS. 20 and 21.

Referring to FIG. 23 (a), when the user enters the operation mode (5700) by directly touching the display unit (570″), the display unit (570″) may display the sleep mode (5750) and other modes. In this case, the user may select the sleep mode (5750) to operate the indoor unit (500″) in the sleep mode.

On the one hand, if no device is wirelessly connected to the indoor unit (500″), a screen for adding a device (e.g., a user terminal) to wirelessly connect to the indoor unit (500″) may be displayed on the display unit (570″), as shown in FIG. 23 (b). The user may touch the ‘add connected device’ button to connect the desired device.

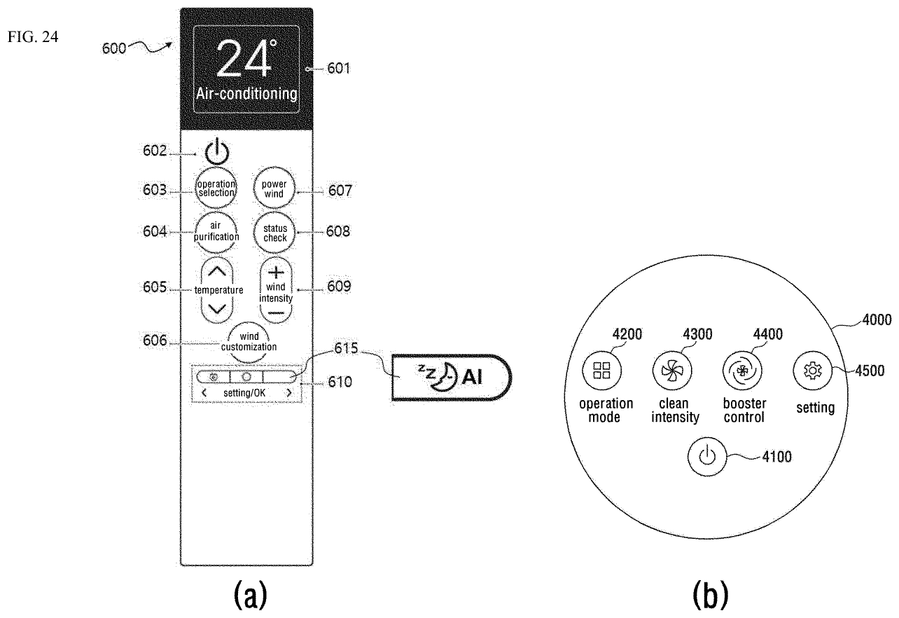

FIG. 24 (a) is a diagram to illustrate a method of operating the indoor units (500, 500″, 500″, 500′″) shown in FIGS. 18 to 22 in sleep mode through the remote control (600).

Referring to FIG. 24 (a), the air conditioner including the indoor units (500′, 500″, 500″, 500″″) shown in FIGS. 18 to 22 may further include a remote control (600) capable of controlling the indoor units (500′, 500″, 500″, 500″′″).

The remote control (600) includes an indicator (601) for displaying the currently selected function and operation state, and a power button (602) for turning the power on and off. The remote control (600) may further include various buttons. For example, it may include a operation selection button (603) for selecting a desired operating mode, an air purification button (604) for keeping the air in the room clean and comfortable, a temperature adjustment button (605) for adjusting a desired temperature, a wind customization button (606) for selecting a wind that is appropriate for the situation and space, a power wind button (607) for quickly adjusting the room temperature by emitting a strong wind, a status check button (608) for checking the operation status of the indoor unit and the indoor environment, a wind intensity button (609) for setting the intensity of the wind, and a setting unit (610) for setting various functions.

The remote control (600) may further include an AI sleep button (615) that may operate a sleep mode. The user may press the AI sleep button (615) to operate the indoor unit (500′, 500″, 500′″, 500″″) into sleep mode.

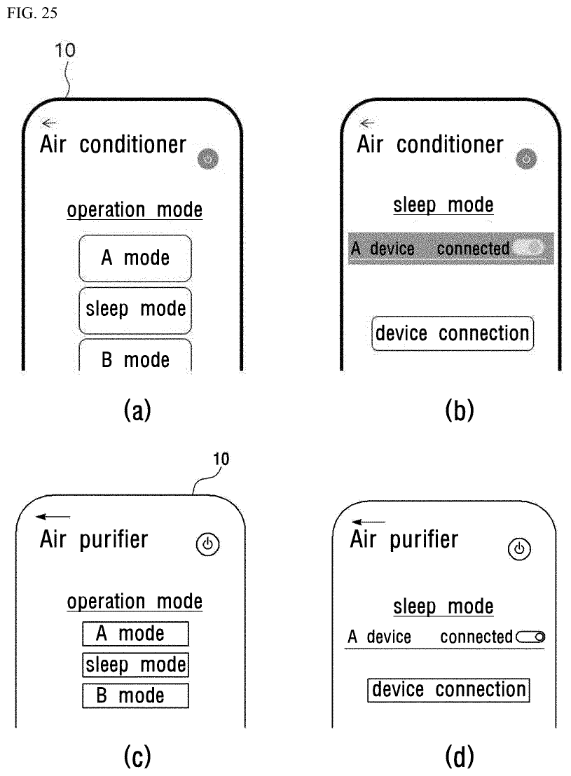

FIGS. 25 (a) and (b) are diagrams to illustrate how to drive the indoor units (500′, 500″, 500″, 500″″) shown in FIGS. 18 to 22 into sleep mode through the user terminal (10). Specifically, FIGS. 25 (a) and (b) depict screens of a first application that remotely controls the indoor units (500′, 500″, 500′″, 500″″) from the user terminal (10).

Referring to FIG. 25 (a), the first application for controlling the indoor units (500′, 500″, 500″, 500″′″) may be installed and stored on the user terminal (10). The user may control the operation of the indoor unit (500′, 500″, 500″, 500″′″) through the first application installed on the user terminal (10).

The first application may provide a screen to control the operation mode of the indoor unit (500′, 500″, 500′″, 500″″) and the user may select a desired operation mode (A mode, sleep mode, B mode, etc.) through the user terminal (10).

In FIG. 25 (a), if the user selects sleep mode, the indoor units (500, 500″, 500″″, 500″″) may be operated in the sleep mode. Here, if the indoor unit (500′, 500″, 500′″, 500″″) is not yet connected to the user terminal (10), a screen may be displayed to specifically set or control the sleep mode of the indoor unit (500′, 500″, 500′″, 500″″), as shown in FIG. 25 (b). This allows the user to select the user terminal (device A) to be connected to the indoor unit (500′, 500″, 500′″, 500″″), and to connect other terminals to the indoor unit (500′, 500″, 500′″, 500″″).

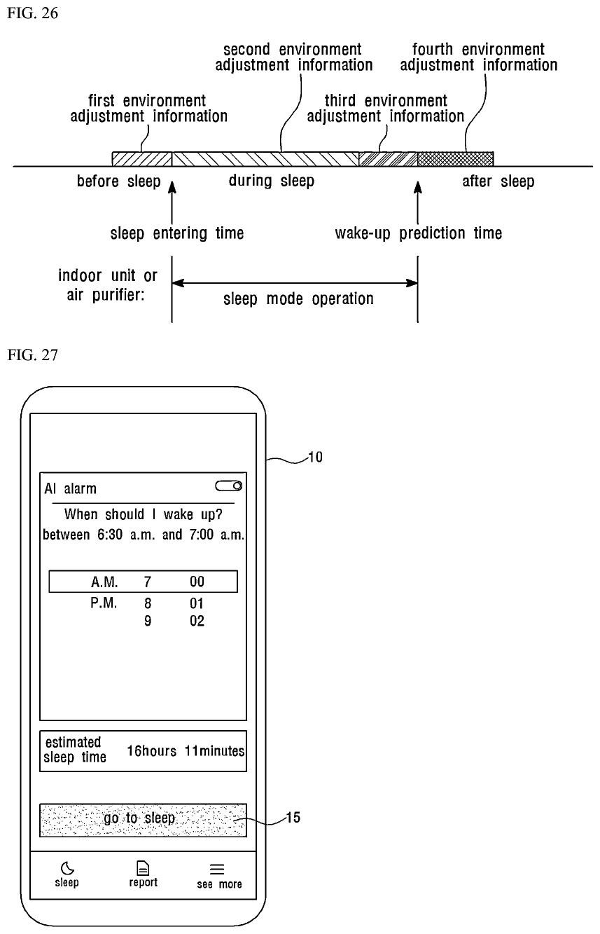

The method of operating the sleep mode shown in FIGS. 23 to 25 above is configured to operate the sleep mode according to the user's selection, but is not limited thereto. According to another embodiment of the present disclosure, the air conditioner may be configured to automatically operating the sleep mode without the user selection. This will be described below with reference to FIG. 26.

FIG. 26 is a diagram to illustrate how an indoor unit or air purifier may be automatically operated into sleep mode.

Referring to FIG. 26, the air conditioner (500), including an indoor unit (500′, 500″, 500″, 500″) may be automatically operated to sleep mode without any user selection according to environment adjustment information generated based on the sleep state information and/or the sleep stage information described above.

For example, if the processor (130) of the computing device (100) of FIG. 16 (a) or the processor (5300) of the air conditioner of FIG. 16 (b) and FIG. 17 (a) generates the second environment adjustment information by identifying from the second sleep adjustment information that the user is entering sleep, i.e., the sleep entering time, the indoor unit (500′, 500″, 500″, 500″) may be configured to automatically initiate the sleep mode.

Further, if the processor (130) of the computing device (100) of FIG. 16 (a) or the processor (5300) of the air conditioner of FIG. 16 (b) and FIG. 17 (a) generates the third environment adjustment information by identifying a wake-up prediction time, the indoor unit (500′, 500″, 500″, 500″) may be configured to automatically terminate the sleep mode at the wake-up prediction time.

Unlike as shown in FIG. 26, the start and end points of the sleep mode may be different, for example, the start point of the sleep mode may be a sleep induction time prior to the sleep entering time.

In another example, as shown in FIG. 25 (a), the time of the sleep mode may be immediately after the first application sleep mode is selected by the user or after a predetermined time period after the sleep mode is selected. Or, as shown in FIG. 25 (b), the time of the sleep mode may be immediately after the predetermined device (device A) is connected to the indoor unit (500′, 500″, 500″, 500″) or after a predetermined time period after the connection.

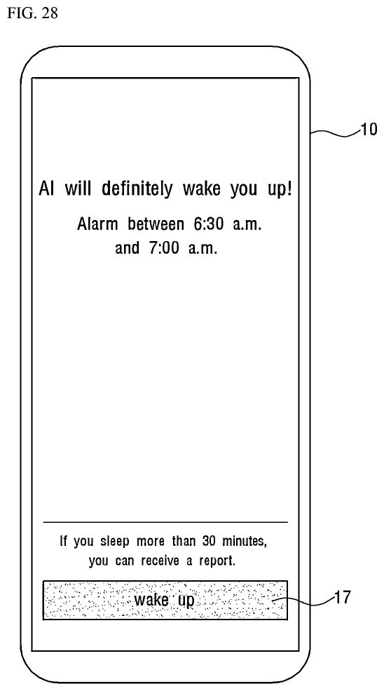

Referring to FIG. 27 and FIG. 28, further examples of when the above sleep mode may occur will be described.

FIG. 27 and FIG. 28 are diagrams to illustrate the time of the sleep mode operation of the indoor unit or air purifier shown in FIG. 26.

As shown in FIG. 27, the time of the sleep mode may be the time when the ‘go to sleep’ button (15) is selected through a second application installed on the user terminal (10). Here, the second application may be an application that measures environment sensing information (e.g., the user's breathing) and automatically to automatically outputs an AI-based sleep report. The second application may be linked with the first application shown in FIGS. 25 (a) and (b) to utilize each other's data.

Alternatively, referring to FIG. 27, the start point of the sleep mode may be after a predetermined time elapses after the ‘go to sleep’ button (15) was selected through the second application. Here, the predetermined time may be a time during which a result measured by an accelerometer sensor provided on the user terminal (10) remains constant.

Again, referring to FIG. 26, the end point of the sleep mode may be after a preset predetermined time elapses, for example, after the time of wake-up prediction.

As another example, referring to FIGS. 25 (a) and (b), the end point of the sleep mode may be immediately after the first application installed on the user terminal (10) and the air conditioner are disconnected from each other.

In another example, referring to FIG. 28, the end point of the sleep mode may be when the “wake up” button (17) is selected through the second application installed on the user terminal (10).

In addition, the start and end points the sleep mode may be varied by the user or manufacturer of the indoor unit (500′, 500″, 500″, 500″).

For continuous operation of the above sleep mode, in the case of the system configuration in FIG. 16 (a), it is preferable that the user terminal (10) is connected through a network and computing device (100), and the computing device (100) is connected through a network with the air conditioner (500). On the one hand, in the case of the system configuration of FIG. 16 (b), it is preferable that the user terminal (10) is connected with the air conditioner (500) through a network or a short-range wireless communication.

### Air Purifier

### Operations of Air Purifier

Hereinafter, an air purifier according to the present invention will be described in detail. FIGS. 16 (c) and (d) are conceptual diagrams to illustrate the operation of an air purifier according to the present invention. Specifically, FIG. 16 (c) is a schematic diagram in which the environment adjustment device (30) of FIG. 1 (a) is implemented as the air purifier (700), and FIG. 16 (d) is a schematic diagram in which the air purifier (700) operates in conjunction with the user terminal (10).

As shown in FIG. 16 (c), the air purifier (700) according to the present invention may operate in conjunction with the user terminal (10) and the computing device (100).

The computing device (100) may include a network unit (110), a memory (120), and a processor (see FIG. 2). The network unit (110) transmits and receives data to and from the user terminal (10), the external server (20) and the air purifier (700). The network unit (110) may transmit and receive data or the like to and from other computing devices, servers etc. for performing a sleep environment adjustment method according to the sleep state information according to one embodiment of the present disclosure. In other words, the network unit (110) may provide a communication function between the computing device (100) and the user terminal (10), the external server (20), and the air purifier (700). For example, the network unit (110) may receive sleep test records and electronic health records for a plurality of users from a hospital server. In another example, the network unit (110) may receive environment sensing information related to a space in which the user is active from the user terminal (10). In another example, the network unit (100) may transmit air quality-related environment adjustment information to the air purifier (700) to adjust the environment of the space where the user is located. Here, the operation, hardware configuration, and software configuration of the network unit (110), memory (120), and processor (130) are the same as described above, so redundant descriptions are omitted.

In addition, the operation, hardware configuration, software configuration, and sleep analysis model of the processor (130) are the same as described above, so redundant descriptions will be omitted. The processor (130) may acquire sleep state information and environment sensing information of the user, as described above.

The processor (130) may generate the first to n environment adjustment information. Specifically, the first environment adjustment information may be generated to control the air purifier to remove fine dust and harmful gases in advance. The first environment adjustment information may include information such as controlling the air purifier to generate noise (white noise) at a level that may induce sleep just before sleeping, adjusting the blowing intensity to a preset level, or lowering the intensity of the LED. In addition, the first environment adjustment information may include information for controlling the air purifier to perform dehumidification/humidification based on temperature and humidity information.

In addition, the processor (130) may generate the second environment adjustment information to turn off the LEDs of the air purifier, operate the air purifier at a noise below the preset level, adjust the blowing intensity below preset intensity, adjust the blowing temperature to a preset range, or maintain the humidity in the sleeping space at a predetermined temperature to control the air purifier based on the second sleep state information.

In addition, the processor (130) may generate the third environment adjustment information and the fourth environment adjustment information based on the third sleep state information and the fourth sleep state information as described above.

As shown in FIG. 16 (d), the air purifier (700) according to an embodiment of the present disclosure may operate in conjunction with the user terminal (10). The air purifier (700) according to an embodiment of the present disclosure may include the configuration of the computing device (100) of FIG. 16 (c) and additional components for operating as the air purifier.

FIG. 17 (b) is a block diagram to illustrate a configuration of an air purifier according to the present disclosure. As shown in FIG. 17 (b), the air purifier (700) according to the present disclosure may include a network unit (710), a memory (720), a processor (730), an operating unit (740), and a measurement unit (750).

The air purifier (700) may be implemented as an air purifier embedded in a ceiling or exterior wall of a building, apartment, or house. The air purifier (700) may also be implemented as a stationary air purifier that is fixed to one side of an indoor space. The air purifier (700) may be implemented as a portable air purifier that is easy to carry and move. The air purifier (700) may also be implemented as an in-car air purifier disposed in a vehicle. The air purifier (700) may be implemented as a wearable air purifier that is worn on the body to purify the air quality around the user.

The air purifier (700) may be implemented as various types of air purifier such as a dust collection filter type air purifier that removes dust using pretreatment and heap filter, an adsorption filter type that adsorbs harmful gases using activated carbon, a wet type that removes dust or harmful gases using water, an electrostatic precipitation type that removes dust using high voltage, anionic type that removes dust by generating negative ions with high voltage and supplying them to air, plasma type that removes harmful gases buy generating positive/negative ions with plasma, and UV photocatalytic type that removes odors and harmful gases by oxidizing/reducing OH radicals and free radicals generated by irradiating TiO with UV light. The air purifier (700) may also be a combination air purifier that utilizes a combination of two or more methods.

The function, operation, hardware configuration, and software configuration of the network unit (710), memory (720), and processor (730) of the air purifier (700) are described above. The first to n environment adjustment information generated by the processor (730) may be transmitted to the operating unit (740). The operating unit (740) may operate various hardware elements of the air purifier (700).

The measurement unit (750) may include one or more sensors for sensing air composition, light intensity, and air purifier component condition within the space. Specifically, the measurement unit may include a dust sensor to detect invisible suspended particles such as PM 1.0, PM 2.5, PM 10, a gas sensor for detecting indoor harmful gases or odors, an illuminance sensor for detecting indoor illumination, a TVOC sensor for measuring the total concentration of about 300 volatile organic compounds included in indoor air, a radon sensor for measuring the concentration of radon, a pressure sensor for measuring filter differential pressure over the life of the filter unit to indicate when to replace the filter, and a temperature sensor for measuring indoor temperature.

Although not shown, the air purifier (700) may comprise a housing with an outlet and an inlet, a filter unit, a blower fan, a sterilization unit, a humidification unit, a heating unit, a cooling unit, a measurement unit etc. The housing may be designed in various ways depending on the implementation of the air purifier (700), such as buried, fixed, mobile, vehicle, wearable etc. The filter unit may be selected in accordance with an air purification method, such as dust collection filter, adsorption filter, wet, electrostatic precipitation, anionic, plasma, UV photocatalytic, etc. The blower fan may be connected to a motor rotated by the power supplied from the power supply. The sterilization unit has the function of sterilizing the inhaled air using chemical or electrical methods. The humidifying unit has the function of humidifying the inhaled air and transmitting it. The heating and cooling units have the function of heating or cooling the inhaled air to a predetermined temperature.

The hardware elements of the air purifier (700) described above are only one embodiment. Some of the hardware elements may be integrated and implemented in one configuration, some may be omitted, and various configurations may be added to perform air purification functions not described above.

Meanwhile, the environment sensing information may be acquired through the user terminal (10). The environment sensing information may be sleep sound information acquired in a bedroom where the user sleeps.

Further, the environment sensing information may be air quality information in the sleeping space acquired from a measurement unit (750) provided in the air purifier (700). The environment sensing information acquired through the user terminal (10) or the measurement unit (750) may be information that is the basis for acquiring the user's sleep state information in the present invention.

As a specific example, the environment state information related to whether the user is before sleep, during sleep, or after sleep may be acquired through the environment sensing information acquired related to a user's activity. In addition, information regarding ambient air quality before, during and after the user's sleep may be acquired.

The processor (730) may acquire the sleep state information based on the environment sensing information acquired through the user terminal (10) and/or the measurement unit (750).

Specifically, the processor (730) may identify singularities in the environment sensing information where information of a preset pattern is detected. Here, the information of the preset pattern may be breathing and movement patterns related to sleep. For example, in the wake state, breathing patterns may be irregular and body movements may be high because all nervous systems are active. Also, the throat muscles are not relaxed, so breathing sounds may be very low. On the other hand, when the user is sleeping, the autonomic nervous system is stabilized, and the breathing pattern may become more regular with less body movement, and the breathing sounds may become louder. In other words, the processor (730) may identify as a singularity in the environment sensing information when a preset pattern of sound information related to regular breathing, less body movement, or less breathing sound is detected. Further, the processor (730) may acquire sleep sound information based on the environment sensing information acquired based on the identified singularity. The processor (730) may identify a singularity in the time-series acquired environment sensing information regarding a time when the user is sleeping, and may acquire the sleep sound information based on the identified singularity.

In addition, the air quality measured by the measurement unit (750) has a significant impact on the user's sleep. According to papers analyzing the relationship between air quality and sleep, it is found that sleep disorders are statistically significantly related to air pollution. For example, exposure to PM 10 may cause difficulty maintaining sleep, especially men are most likely to experience sleep disturbance when exposed to PM 1. Women are most likely to experience sleep disturbance when exposed to PM 1 and PM 2.5. It is also found that sleep disturbance related to wheezing are most like to occur when SO2 and O3 are high. It is also found that pregnant women exposed to PM 2.5 between 31 and 35 weeks of gestation are most likely to have shorter sleep duration. Various studies have been conducted on the relation between AHI and other measures of air quality. While the results vary slightly from study to study, the conclusion that air quality and sleep are highly correlated is the same.

The air purifier (700) according to an embodiment of the present disclosure may acquire sleep state information based on environment sensing information, generate environment adjustment information, and use it to perform actions appropriate for sleep stages.

Specifically, the processor (730) of the air purifier (700) may generate the first environment adjustment information for controlling the air purifier from a time when the user is predicted to be preparing to sleep (e.g., a sleep induction time) to a time when the user falls asleep (i.e., when the second sleep state information is acquired), if the user's condition is determined to be a pre-sleep state. The first environment adjustment information may be generated by reflecting PM concentration, harmful gas concentration, CO2 concentration, SO2 concentration, O3 concentration, humidity, temperature, etc. measured by the measurement unit (750).

The first environment adjustment information may include information for controlling the air purifier to remove fine dust and harmful gases in advance by a predetermined time (e.g., 20 minutes before the user's sleep), information for controlling the air purifier to generate noise (white noise) at a level that may induce sleep just before sleep, information for controlling the air purifier to adjust the blowing intensity below a preset intensity or lower the power of the LEDs, information for controlling the air purifier to execute dehumidification/humidification based on temperature and humidity information in the sleeping space, etc.

In addition, the processor (730) may generate the second environment adjustment information to turn off the LEDs of the air purifier based on the second sleep state information, operate the air purifier at a noise level below a preset level, adjust the blowing intensity below a preset intensity, adjust the blowing temperature to a preset range, or maintain the humidity in the sleeping space at a predetermined temperature.

The second environment adjustment information is based on the second sleep state information, and may be control information for controlling the air purifier to turn off the LED of the air purifier, operate the air purifier at a noise level below a preset level, adjust the blowing temperature to a preset range, or maintain the humidity in the sleeping space at a predetermined temperature. The user may be induced to sleep by airflow, white noise, etc. in the sleeping space where fine dust and harmful gases are removed just before sleeping, and after entering sleep, the user may get a good night's sleep with optimal temperature, humidity, etc. controlled.

### Configurations of Air Purifier According to Embodiments

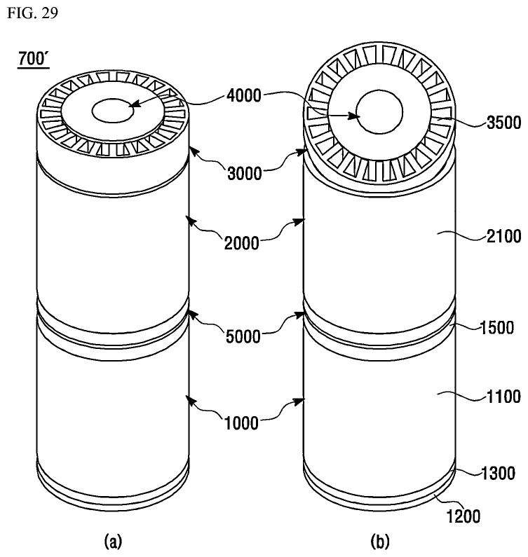

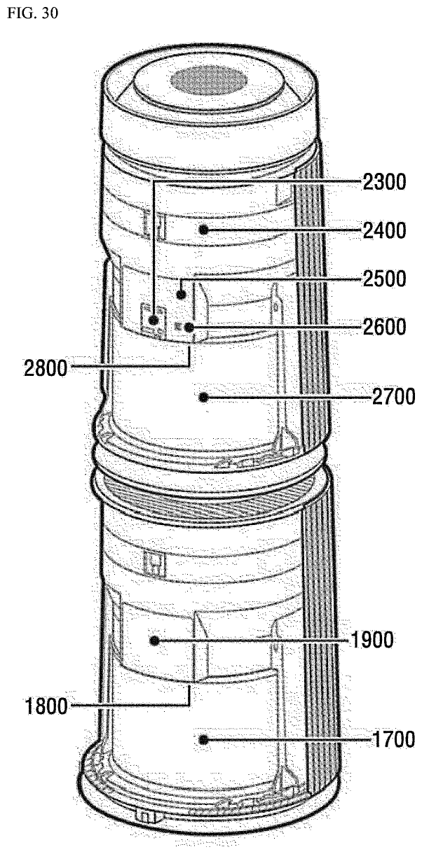

FIGS. 29 (a) and (b) are diagrams to illustrate one example of the air purifier shown in FIG. 16 and FIG. 17.

Referring to FIGS. 29 (a) and (b), the air purifier (700′) according to one example of the present invention includes a blowing device (1000, 2000) for generating an air flow and a flow diverter (3000) for switching a output direction of the air flow generated by the blowing device (1000, 2000).

The blowing device (1000, 2000) includes a first blowing device (1000) generating a first air flow and a second blowing device (2000) generating a second air flow. Hereinafter, the blowing devices (1000, 2000) may also be defined as “air purification modules”.

The first blowing device (1000) and the second blowing device (2000) may be arranged in an upward or downward direction. For example, the second blowing device (2000) may be disposed on the upper side of the first blowing device (1000). In this case, the first air flow forms a flow that draw in indoor air present on the lower side of the air purifier (700″), and the second air flow forms a flow that draw in indoor air present on the upper side of the air purifier (700′).

The air purifier (700′) includes a cover (1100, 2100) forming an exterior.

The covers (1100, 2100) include a first cover (1100) forming an exterior of the first blowing device (1000). The first cover (1100) may have a cylindrical shape. And, an upper portion of the first cover (1100) may be configured to have a smaller diameter than a lower portion. In other words, the first cover (1100) may have a conical shape with a truncated end.

The first cover (1100) may comprise at least two or more parts. The parts may be coupled or separated from each other. When at least one of the parts is rotated, the first cover (1100) opens and may be separated from the air purifier (700′). The portion where the parts are coupled may be provided with a jamming device. The jamming device may include a snag or a magnetic member. By opening the first cover (1100), the internal components of the first blowing device (1000) may be replaced or repaired.

In the first cover (1000), a first inlet may be formed through which air is drawn in. The first inlet may comprise a through hole formed which at least a portion of the first cover (1100) is penetrated. The first inlet may be formed in a plurality.

The plurality of the first inlet may be evenly formed circumferentially along the outer peripheral surface of the first cover (1100) to allow air to be drawn in any direction relative to the first cover (1100). In other words, air may be drawn in a 360-degree direction relative to a centerline in an upward or downward passing through the inner center of the first cover (1100).

In this way, by configuring the first cover (1100) as a cylindrical shape and forming a plurality of first inlets along the outer circumferential surface of the first cover (1100), the amount of air to be drawn in may be increased.

The air drawn through the first inlet may flow in an approximately radial direction from the outer peripheral surface of the first cover (1100). Relative to FIG. 29, an up and down direction is defined as axial direction, and a transverse direction is defined as a radial direction. The axial direction may correspond to the direction of the center axis of a fan disposed inside the air purifier (700′) that generates air flow, that is, the direction of the motor shaft of the fan. The radial direction may be understood as a perpendicular direction to the axial direction. And, the circumferential direction is understood as an imaginary circular direction formed when rotating with the axial direction as the center and the distance of the radial direction as the rotation radius.

The first blowing device (100) further includes a base (1200) provided on the lower side of the first cover (1100) and resting on the ground. The base (1200) is spaced apart downwardly from the bottom of the first cover (1100). And in the spacing between the first cover (1100) and the base (1200), a base inlet (1300) may be formed. Air may be drawn through the base inlet (1300), and the drawn air may enter the first blowing device (1000).

The first blowing device (1000) may have a plurality of inlets, such as the first inlet and the base inlet (1300). Air present in the lower part of the indoor space may be easily drawn into the first blowing device (1000) through the plurality of inlets. Thus, the amount of air drawn in may be increased.

On top of the first blowing device (1000), a first outlet (1500) may be formed. Air output through the first outlet (1500) may flow in an axial upward direction.

The covers (1100, 2100) may include a second cover (2100) forming an exterior of the second blowing device (2000). The second cover (2100) may be cylindrical in shape. And, the upper portion of the second cover (2100) may be configured to have a smaller diameter than the lower portion. In other words, the second cover (2100) may have a conical shape with a truncated end.

The second cover (2100) may comprise at least two or more parts. The parts may be coupled or separated from each other. When at least one of the parts rotates, the second cover (2100) opens and may be separated from the air purifier (700′). The portion where the parts are coupled may be provided with a jamming device. The jamming device may include a jamming element or a magnetic element. By opening the second cover (2100), the internal components of the second blower (2000) may be replaced or repaired.

The bottom diameter of the second cover (2100) may be formed smaller than the top diameter of the first cover (1100). Thus, in terms of the overall shape of the covers (1100, 2100), the lower cross-sectional area of the covers (1100, 2100) is formed larger than the upper cross-sectional area. Accordingly, the air purifier (700′) may be stably supported on the ground.

In the second cover (2100), the second inlet may be formed through which air is drawn. The second inlet may comprise a through hole formed by at least a portion of the second cover (2100) being penetrated. The second inlet may be formed in a plurality.

The plurality of the second inlet may be formed circumferentially evenly along the outer peripheral surface of the second cover (2100) to allow air to be drawn in any direction relative to the second cover (2100). In other words, air may be drawn in a 360-degree direction relative to a centerline in an upward or downward direction passing through the inner center of the second cover (2100).

In this way, by configuring the second cover (2100) as a cylindrical shape and forming a plurality of second inlets along the outer circumferential surface of the second cover (2100), the amount of air to be drawn in may be increased.

The air drawn in through the second inlets may flow approximately radially from the outer peripheral surface of the second cover (2100).

The air purifier (700′) includes a compartment device (5000) provided between the first blowing device (1000) and the second blowing device (2000). By the compartment device (5000), the second blowing device (2000) may be positioned spaced apart from the first blowing device (1000).

The compartment device (5000) may guide air outlet from the first outlet (1500). For example, the compartment device (500) may guide air output axial direction from the first outlet (1500) to output in transverse direction perpendicular to axial direction.

The flow diverter (3000) may be installed on the upper side of the second blowing device (2000). Based on the air flow, the air passage of the second blowing (2000) may be in communication with the air passage of the flow diverter (3000). Air passing through the second blowing device (2000) may be output to the outside through the second outlet (3500), via the air passage of the flow diverter (3000). The second outlet (3500) may be formed at the top of the flow diverter (3000).

The flow diverter (3000) may be movably configured. Specifically, the flow diverter (3000) may be in a lying state (first position) as shown in FIG. 29 (a), or in an inclined upright state (second position) as shown in FIG. 29 (b). Hereinafter, the flow diverter (3000) may also be named as a “circulator”.

On top of the flow diverter (3000), a display unit (4000) may be disposed, which includes an operation unit for displaying operation information of the air purifier (700′) and controlling operation of the air purifier (700′). The display unit (4000) may move together with the flow diverter (3000).

FIG. 30 is a diagram depicting a portion of the covers (1100, 2100) of the air purifier (700′) removed shown in FIG. 29.

Referring to FIG. 29 and FIG. 30, the air purifier (700′) includes a sensor (2300) that detects a concentration of dust. The sensor (2300) may be disposed in the second blowing device (2000), but it not limited to it, and may also be disposed in the first blowing device (1000). The sensor (2300) may include a sensor that sense ultra-fine particulate matter (PM 1.0).

The air purifier (700′) may include a smart diagnostic unit (2400). The smart diagnostic unit (2400) may check the product condition through smart diagnosis if the air purifier (700′) is malfunctioning or broken.

The air purifier (700′) may include an AI sensor communication module (2500). The AI sensor communication module (2500) may be in conjunction with an AI sensor (not shown) that may be in communication with the air purifier (700′) to detect the location of contamination.

The air purifier (700′) may include a gas sensor (2600). The gas sensor (2600) may detect gases or odors.

The air purifier (700′) may include a filter (1700, 2700) to purify the air. The filters (1700, 2700) may be disposed in each of the first blowing device (1000) and the second blowing device (2000).

The air purifier (700′) may include a filter condition detection sensor (1900). The filter condition detection sensor (1900) may detect replacement time of the filters (1700, 2700).

The air purifier (700′) may include a UV light emitting element (1800, 2800) for sanitizing a fan disposed therein. Each of the first and second blowing devices (1000, 2000) may have a fan inside, and the UV light emitting elements (1800, 2800) may be positioned to illuminate the fan inside each of the blowing devices (1000, 2000).

FIG. 24 (b) is a diagram depicting an example of a display unit (4000) of the air purifier (700′) according to one embodiment of the present disclosure. Referring to FIG. 24 (b), the display unit (4000) may include a plurality of control units (4100, 4200, 4300, 4400, 4500).

The plurality of control units (4100, 4200, 4300, 4400, 4500) may include a start/stop button (4100) to start or stop the air purifier (700′), an operating mode button (4200) to select an operating mode, a clean intensity button (4300) to adjust the wind intensity of the air purifier (700′), a booster control button (4400) to adjust the intensity and rotation settings of the booster, and a setting button (4500) to set management and notification settings for the air purifier (700′). These multiple control units (4100, 4200, 4300, 4400, 4500) may be touch sensor or mechanical buttons.

Control signals input through the plurality of operation units (4100, 4200, 4300, 4400, 4500) of the display unit (4000) are input to the processor (730) shown in FIG. 17 (b), and the processor (730) may control the operation unit (740) based on the input control signals.

The operation mode (4210) may include various driving modes. For example, it may include an AI mode, a pet mode, a clean booster mode, a dual clean mode, a single clean mode, etc. Here, the AI mode may be a mode that automatically adjusts the operation mode and the cleaning intensity according to the overall cleanliness of the air purifier (700′) and the AI sensor. The pet mode may be a customized mode for users with pets. The clean booster mode may be a mode that circulates indoor air by quickly sending purified air to a long distance using a booster provided in the second blower device (2000). The dual clean mode may be a mode in which the first blowing device (1000) and the second blowing device (2000) operate simultaneously to quickly clean the indoor air. The single cleaning mode may be a mode in which the indoor air is cleaned by the second blowing machine (2000).

The air purifier (700′) according to an embodiment of the present disclosure may further include a sleep mode as the operation mode (4210). This is illustrated with reference to (c) and (d) of FIG. 23.

FIGS. 23 (c) and (d) are diagrams of the display unit (4000) to illustrate the sleep mode of the air purifier (700′) according to an embodiment of the present disclosure.

Referring to (c) of FIG. 23, the operation mode (4210) may include a sleep mode (4250). The sleep mode (4250) is a mode that detects a user's sleep state and automatically adjusts the air quality within the user's sleeping space. The sleep mode (4250) may be controlled by the processor of the air purifier (700″).

Based on the first environment adjustment information or the n environment adjustment information generated by the computing device (100) in FIG. 16 (c) or the air purifier (700′) in FIG. 16 (d), the air purifier (700′) may perform air quality adjustment within the sleeping space.

For example, the air purifier (700′) may remove fine dust and harmful gases in advance by a predetermined time before the user's sleep (e.g., 20 minutes) according to the first environment adjustment information. Alternatively, the air purifier (700′) may generate noise (white noise) at a level that may induce sleep just before sleep, adjust the blowing intensity below a preset level, or reduce the brightness of the display unit (4000). The air purifier (700′) may turn off the display unit (4000), operate at a noise below a preset level, adjust the blowing temperature to a preset range, or maintain the humidity in the sleeping space at a predetermined temperature based on the second environment adjustment information. The air purifier (700′) may lower the blowing intensity and noise at the wake-up time, generate white noise to gradually induce wake-up, keep the noise below a preset level, or operate in conjunction with a wake-up prediction time or wake-up recommendation time according to the third environment adjustment information. The air purifier (700′) may control at least one of the blowing intensity, noise level, and alarm based on the fourth environment adjustment information.

In the case of the system configuration of FIG. 16 (c), in order for the air purifier (700′) to operate in sleep mode (4250), the air purifier (700′) may undergo a device connect process to connect with the computing device (100) and a network.

In the case of the system configuration of FIG. 16 (d), in order for the air purifier (700′) to operate in sleep mode (4250), the air purifier (700′) may perform a connection process for linking with the user terminal (10) as shown in FIG. 23 (d). The air purifier (700′) and the user terminal (10) may be connected wirelessly. For example, the air purifier (700′) and the user terminal (10) may be directly connected through a network, as shown in FIG. 16 (d).

### Configurations of the Air Purifier According to Another Embodiment

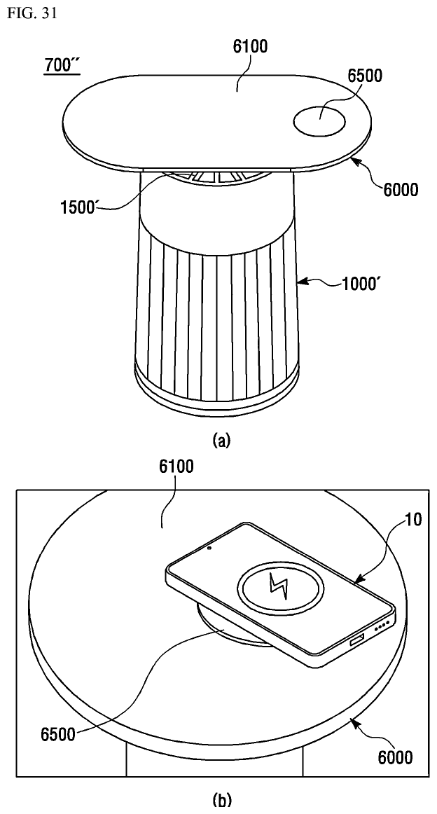

FIG. 31 (a) is a diagram to illustrate another example of the air purifier shown in FIG. 16 and FIG. 17.

The air purifier (700″) shown in FIG. 31 (a) may be an air purifier that may be used in an individual space, such as a bedroom or study.

The air purifier (700″) includes a blowing device (1000″) and a table (6000).

The blowing machine (1000′) is a corresponding configuration to the first blowing machine (1000) shown in FIG. 29, includes configurations for purifying air quality internally like the first blowing device (1000). Accordingly, the detailed description of the blowing device (1000′) is replaced by the description of the first blowing device (1000) shown in FIG. 29.

A table (6000) may be disposed on the blowing device (1000′).

The table (6000) may include a bottom surface (not shown) that guides air exiting from the outlet (1700′) disposed at the top of the blowing machine (1000′), a top surface (6100) on which items may be placed, and a wireless charging unit (6500) disposed at a portion of the top surface (6100). A lighting unit (not shown) capable of emitting light of various colors may be disposed on the bottom surface (not shown) of the table (6000).

The air purifier (700″) may operate in a sleep mode to automatically adjust the air quality within the sleeping space, such as the air purifier (700″) shown in FIG. 29.

The sleep mode of the air purifier (700″) may perform air quality adjustment in the sleeping space based on the first environment adjustment information or the n environment adjustment information generated by the air purifier (700″), such as the computing device in FIG. 16 (c) or FIG. 16 (d).

For example, the air purifier (700″) may remove fine dust and harmful gases in advance by a predetermined time before the user's sleep (e.g., 20 minutes) according to the first environment adjustment information. Alternatively, the air purifier (700″) may generate noise (white noise) at a level that may induce sleep just before sleep, adjust the blowing intensity below a preset level, or reduce the brightness of the lighting unit (not shown). The air purifier (700″) may turn off the lighting unit (not shown), operate at a noise level below a preset level, adjust the blowing intensity below a preset intensity, adjust the blowing temperature to a preset range, or maintain the humidity in the sleeping space at a predetermined temperature according to the second environment adjustment information. The air purifier (700″) may lower the blowing intensity and noise at the wake-up time, generate white noise to gradually induce wake up, keep the noise below a preset level, or operate in conjunction with a wake-up prediction time or wake-up recommendation time based on the third environment adjustment information. The air purifier (700″) may control at least one of blowing intensity, noise level, and alarm based on the fourth environment adjustment information.

Unlike the air purifier (700′) shown in FIG. 29, the air purifier (700″) may not include a display unit.

Accordingly, in accordance with the system configuration of FIGS. 16 (c) and (d), the air purifier (700″) may be remotely controlled by the user terminal (10) of FIG. 16 in order for the air purifier (700″) to operate in a sleep mode.

It may be remotely controlled by the user terminal (10) of FIG. 16. This will be described with reference to FIG. 25.

FIG. 25 (c) shows a screen of a first application that remotely controls the air purifier (700″) from the user terminal (10), and FIG. 25 (d) shows a screen of an application that controls a sleep mode of the air purifier (700″).

Referring to FIG. 25 (c), an application for controlling the air purifier (700″) may be installed on the user terminal (10). The air purifier (700″) may be controlled through the application.

The first application may provide a screen for controlling the operation mode of the air purifier (700″), and the user may select a desired operation mode (A mode, sleep mode, B mode, etc.).

If the user selects sleep mode in FIG. 25 (c), a screen for specifically controlling the sleep mode of the air purifier (700″) may be displayed, as shown in FIG. 25 (d). In this way, the user may select a user terminal to connected to the air purifier (700″), and may connect other terminals with the air purifier (700″).

The air purifier (700″) may perform a connection process for linking with the user terminal (10), as shown in FIGS. 25 (c) and (d). The air purifier (700″) and the user terminal (10) may be connected wirelessly. For example, the air purifier (700′) and the user terminal (10) may be connected through a network as shown in FIG. 16 (d).

On the one hand, the control of the air purifier (700″) through the first application shown in FIG. 25 may be applied to the air purifier (700) shown in FIG. 29.

### Sleep Mode Operation Method of the Air Purifier

The air purifier (700′, 700″) described above are configured to operate in sleep mode at the option of the user, but are not limited thereto. An air purifier according to another embodiment of the present disclosure may be configured to automatically operate sleep mode other than the option of the user. This will be described with reference to FIG. 26.

FIG. 26 is a diagram to illustrate the operation of an air purifier (700″) according to another embodiment of the present disclosure.

Referring to FIG. 26, the air purifier (700″″) may be automatically operated in sleep mode without any user selection according to the environment adjustment information generated based on the sleep state information and/or sleep stage information. For example, if the processor (130) of the computing device (100) of FIG. 16 (c) or the processor (730) of the air purifier of FIG. 16 (d) and FIG. 17 (b) generates the second environment adjustment information by identifying a time when the user enters sleep, i.e., sleep entering time, the air purifier (700′″) may be configured to automatically initiate the sleep mode.

Also, if the processor (130) of the computing device (100) in FIG. 16 (c) or the processor (730) of the air purifier in FIG. 16 (d) and FIG. 17 generates the third environment adjustment information by identifying a wake-up prediction time, the air purifier (700′″) may be configured to automatically terminate the sleep mode at the wake-up prediction time.

Unlike as shown in FIG. 26, the start point and the end point of the sleep mode may be different. For example, the start point of the sleep mode may be a sleep induction point prior to the sleep entering point.

As another example, as shown in FIG. 25 (c), the time of the sleep mode may be immediately after the sleep mode of the first application is selected by the user or after a predetermined time after the sleep mode is selected. Or, as shown in FIG. 25 (d), the time of the sleep mode may be immediately after the predetermined device (device A) is connected to the air purifier or after a predetermined time after the connection.

Other examples of when the above sleep mode may occur will be described with reference to the diagrams.

FIG. 27 and FIG. 31 (b) are diagrams to illustrate the time of the sleep mode operation of the air purifier (700′″) shown in FIG. 26.

As shown in FIG. 31 (b), the time of the sleep mode may be immediately after the user terminal (10) is placed on the wireless charging unit (6500) to begin charging or after a predetermined period of time after the user terminal (10) begins charging.

Referring to FIG. 31 (b), the time of the sleep mode may be a predetermined time after the user terminal (10) predetermined portion of the top surface (6100). Here, the predetermined time may be a time during which a result value measured by an accelerometer sensor provided on the user terminal (10) remains constant.

As shown in FIG. 27, the time of the sleep entering mode may be when the “go to sleep” button (15) is selected through a second application installed a second application installed on the user terminal (10). Here, the second application may be an application that measures environment sensing information (e.g., the user's breathing) and automatically outputs an AI-based sleep report.

The second application may be linked with the first application shown in FIGS. 25 (c) and (d) to utilize each other's data.

Alternatively, referring to FIG. 27, the timing of the sleep mode may be after a predetermined period of time after the ‘go to sleep’ button (15) is selected through the second application. Here, the predetermined time may be a time during which the measured of the accelerometer sensor provided on the user terminal (10) remain constant.

Referring again to FIG. 26, the end point of the sleep mode may be after a predetermined period of time after a wake-up prediction, for example.

As another example, referring to FIGS. 25 (c) and (d), the end point of the sleep mode may be immediately after the first application installed on the user terminal (10) and the air purifier are disconnected from each other.

In another example, referring to FIG. 31 (b), the end point of the sleep mode may be immediately after the user terminal (10) moves away from the wireless charging unit (6500) and wireless charging is interrupted, or after a predetermined amount of time elapses after the interruption. Alternatively, the end point of the sleep mode may be immediately after the user terminal (10) moves away from the table (6000) or after a predetermined amount of time after moving away.

As another example, referring to FIG. 28, the end point of the sleep mode may be when the “wake up” button (17) is selected through the second application installed on the user terminal (10).

In addition, the start point and the end point of the sleep mode may be varied by the user or manufacturer of the air purifier (700′″).

For continuous operation of the above sleep mode, in the case of the system configuration of FIG. 16 (c), the user terminal (10) is connected to the computing device and a network, and the computing device (100) is preferably connected to the air purifier (700″″) through a network. On the one hand, in the case of the system configuration of FIG. 16 (d), the user terminal (10) is preferably connected to the air purifier (700″″) through a network or a short-range wireless communication.

The air purifier (700″″) may have the same hardware configuration of the air purifier (700′, 700″) shown in FIG. 29 (a) or FIG. 31.

Sleep Mode and Wake-up Mode Operations of an Air Purifier or Air Purifying Fan

### According to an Embodiment

Further, the operation of the sleep mode and wake-up mode of a tabletop air purifier that regulates air quality and light and an air purifying fan that regulates air quality and temperature is described.

First, here is how a tabletop air purifier works in sleep mode.

The tabletop air purifier may operate on its own, but may also be packaged with an air conditioner, humidifier, and/or dehumidifier for premium air care.

In the bedtime preparation phase, the tabletop air purifier may wirelessly charge the smartphone (900) if the user places the smartphone (900) on the table during certain times when the smartphone (900) is not in use, and may recognize the sleep management application installed on the smartphone (900) to begin measuring the user's sleep.

The air conditioner may regulate a room temperature suitable for the user's sleep environment, and the humidifier and/or dehumidifier may regulate a room humidity suitable for the sleep environment.

In the entering sleep phase, the tabletop air purifier may turn off the indirect light, and in the sleep phase, the sleep track application and the sleep management application may work together to measure the user's sleep situation in real time.

Next, the operations of the tabletop air purifier in wake-up mode are as follows.

In the pre-wake-up phase, the tabletop air purifier may generate intentional noise in power air purification mode to perform wake-up alarm function, operate a smart alarm installed in a healthcare application, or adjust the light intensity to gradually brighten the sleep light.

In the wake-up phase, if the user's smartphone (900) detects a sleep state check, the sleep measurement is terminated, and in the post-wake up phase, the healthcare application may display the analyzed user's sleep report on the user's smartphone (900).

On the one hand, the operation of the air purifying fan in sleep mode is as follows.

Like the tabletop air purifier, the air purifying fan may be operated alone, buy may also be packaged with a humidifier and/or dehumidifier for premium air care.

During the bedtime preparation phase, the air purifying fan may be manually activated through the healthcare application to adjust the temperature for sleep using cool and warm air, and the humidifier and/or dehumidifier may adjust the room humidity suitable for a sleep environment.

During the sleep phase, the sleep track application and the sleep management application may work together to measure the user's sleep situation in real time.

Next, the operation of the air purifier fan in the wake-up mode, i.e., the pre-wake-up phase, the wake-up phase, and the post wake-up phase, is the same as the operation of the tabletop air purifier in the wake-up mode, so the description will be omitted here.

### Specific Scenarios of Smart Home Appliances by Sleep Stage

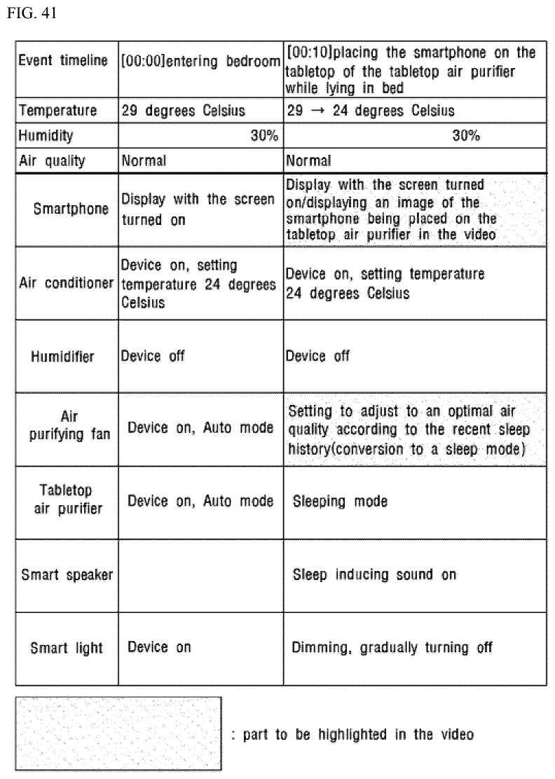

FIG. 41 is a table depicting the operations of the bedtime preparation phase of a specific scenario of smart home appliances operating time series by sleep stage of the user using the sleep analysis method according to the present invention.

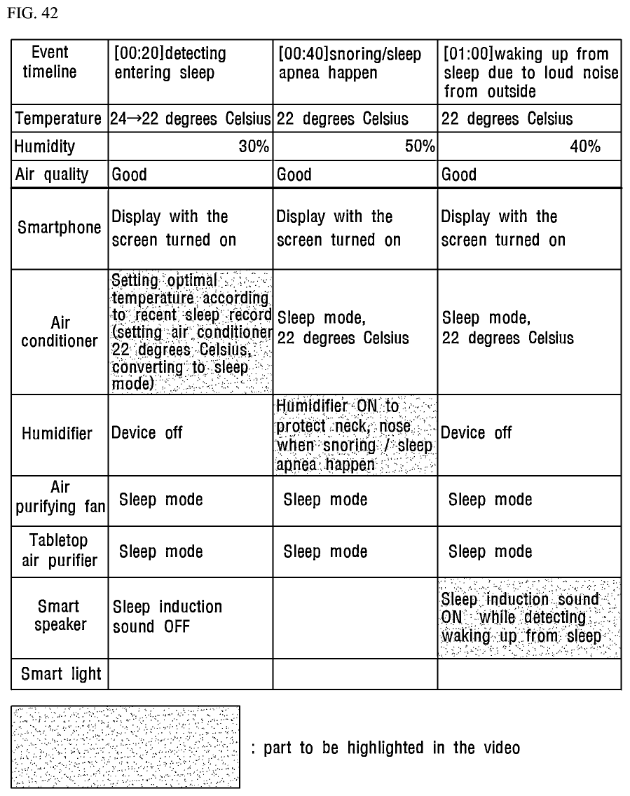

FIG. 42 is a table depicting the operations of the phase from after entering sleep to before deep sleep, which is time-series connected to FIG. 41 in the above scenario.

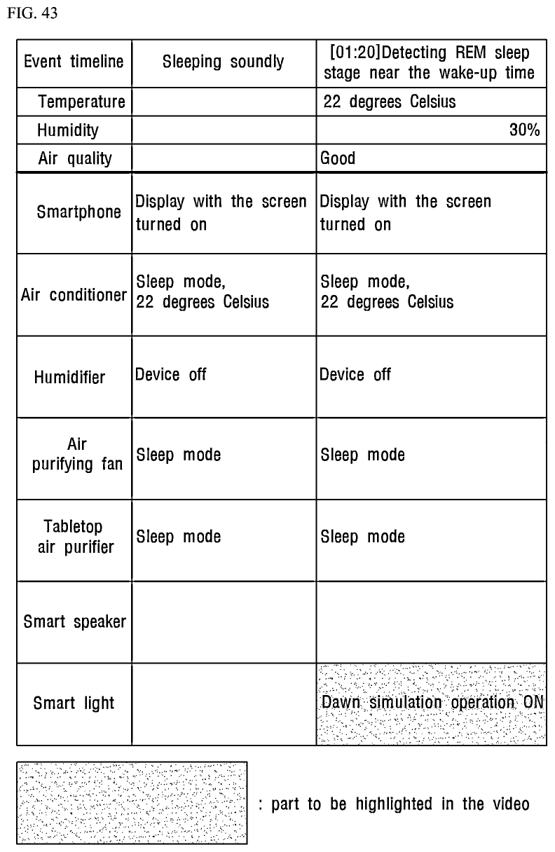

FIG. 43 is a table depicting the operations of the phase from after deep sleep to before wake-up, which is time-series linked to FIG. 42 of the above scenario.

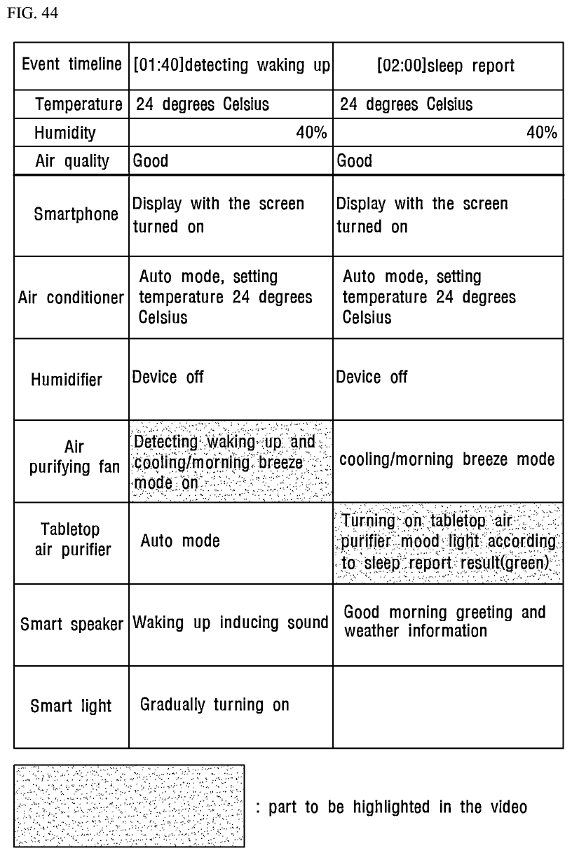

FIG. 44 is a table depicting the operations of the wake-up phase of the above scenario, which is time-series linked to FIG. 43.

Referring to FIGS. 50 and 51 and FIGS. 41 to 44, the following describes the operation of a specific scenario of smart home appliances operating time-series according to the sleep stages of the user using the sleep analysis method of the present invention. However, the scenarios described below are only examples according to the present invention, and the scope of the present invention is not limited thereto.

First, assume that the user enters the bedroom at midnight wakes up at 01:40 AM.

Further, it is assumed that the initial temperature when the user enters the bedroom is 29 degrees Celsius and the initial humidity is 30%, so that the air quality is “normal” at night when the temperature is high but the humidity is not high.

As shown in FIG. 41, at the time the user enters the bedroom (00:00), the smartphone (900) may be displayed with the screen turned on (the same is true in subsequent steps).

In addition, the air conditioner, the air purifying fan, the tabletop air purifier, and the smart light may be turned on, while the humidifier and the smart speaker may remain off. Meanwhile, the air conditioner may be set to an operating temperature of 24 degrees Celsius.

Also, the air purifying fan and the tabletop air purifier may be set to automatic mode.

When the user places the smartphone (900) on the tabletop of the tabletop air purifier while lying in bed (00:10), the screen of the smartphone (900) may display an image of the smartphone (900) being placed on the tabletop air purifier.

Further, the air purifying fan and the tabletop air purifier may be converted to a sleep mode. The air purifying fan may be set to adjust to an optimal air quality according to the user's recent sleep history.

Also, smart speakers may also turn on sleep inducing sounds. The smart light may be set to gradually dim and then turn off.

Meanwhile, the room temperature of the bedroom may decrease from an initial temperature of 29 degrees Celsius to an operating temperature of 24 degrees Celsius by the operation of the air conditioner whose operation temperature is set to 24 degrees Celsius.

As shown in FIG. 42, at a time (00:20) when the user's entering sleep state is detected, the air conditioner may be reset to an optimal temperature (e.g., 22 degrees Celsius) according to the user's recent sleep history, and may converted to sleep mode.

Additionally, the smart speaker may turn off the sleep induction sound, as it is no longer necessary to induce the user to entering sleep.

Meanwhile, as the operating temperature is reset to the optimal temperature of 22 degrees Celsius and the air conditioner is operated, the room temperature in the bedroom may decrease from 24 degrees Celsius toward the optimal temperature of 22 degrees Celsius, and the air quality may be converted to a “good” state by the continued operation of the air purifying fan and the tabletop air purifier that were set to the user's optimal air purification state.

If it is assumed that the snoring and/or sleep apnea during sleep time is happened at time point (00:40), a sound sensor mounted inside the humidifier may detect a change in the user' breathing, and the processor (830) in the humidifier may turn on the humidifier to protect the user's nose. As a result, the initial humidity of 30% may rise to 50%.

Meanwhile, the smart speaker may turn on a sleep induction sound to induce the user to entering back to sleep.

As shown in FIG. 43, through the user's breathing, as detected by an sound sensor mounted inside each of the plurality of the smart home appliances (800), an embedded processor may identify that the user enters a deep sleep state. In this case, the current state of each smart home appliance (800) may be maintained.

When the scheduled wake-up time (01:40) is approaching (01:20), assuming that if the REM sleep stage among the user's sleep stage is detected, the smart light may turn on the dawn simulation operation.

Finally, as shown in FIG. 44, at the scheduled time to wake up (01:40), assuming the wake-up phase is detected during the user's sleep phase, the air conditioner may convert to automatic mode and reset the operating temperature to 24 degrees Celsius.

In addition, the air purifying fan may be set to a cooling and/or morning breeze mode. The tabletop air purifier may be set to an automatic mode.

In addition, the smart speaker may turn on a wake-up call to induce the user to wake up. Smart lights may be set to turn off after the dawn simulation operation as they do not need to be on in the bedroom.

Further, at the time point (02:00), assuming that the user's wake-up state is detected, the colored mood light in the tabletop air purifier may be turned may be turned on to green, etc. Furthermore, the smart speaker may provide the user with the weather information of the day audibly, such as a “good morning” greeting.

The specific figures and operations of the smart home appliances described above are only examples to help understand the contents of the present invention, and the present invention is not limited thereto.

The steps of methods or algorithms described related to embodiments of the present disclosure may be implemented directly in hardware, implemented as software modules executed by hardware, or a combination thereof. The software module may reside in a random access memory (RAM), read only memory (ROM), erasable programmable ROM (EPROM), electrically erasable programmable ROM (EEPROM), flash memory, hard disk, removable disk, CD-ROM, or any other form of computer-readable recording medium well known in the art.

The components of the present invention may be implemented as programs (or applications) and stored on a medium for execution in combination with a computer, which is hardware. The components of the present disclosure may be executed as software programs or software elements. Similarly, embodiments may be implemented in programming or scripting languages such as C, C++, Java, assembler, and the like, including various algorithms implemented as a combination of data structures, processes, routines, or other programming constructs. Functional aspects may be implemented as algorithms executing on one or more processors.

One of ordinary skill in the art of the present disclosure will understand that the various exemplary logic blocks, modules, processors, means, circuits and algorithmic steps described related to the embodiments disclosed herein may be implemented by electronic hardware, various forms of program or design code (for convenience, referred to herein as “software”), or a combination of both. To clearly illustrate this interchangeability of hardware and software, various exemplary components, blocks, modules, circuits, and steps have been generally described above regarding their functions. Whether these features are implemented as hardware or software will depend on design restrictions imposed on a specific application and the overall system. One of ordinary skill in the art of the present invention may implement functions described in various ways for each particular application, but such implementation decisions should not be interpreted as departing from the scope of the present invention.

Various embodiments presented herein may be implemented as methods, devices, or articles of manufacture using standard programming and/or engineering techniques. The term “article of manufacture” includes a computer program, carrier, or medium accessible from any computer-readable device. For example, computer-readable media include magnetic storage devices (e.g., hard disks, floppy disks, magnetic strips, etc.), optical disks (e.g., CDs, DVDs, etc.), smart cards, and flash memory devices (e.g., EEPROMs, cards, sticks, key drives, etc.), but are not limited thereto. In addition, the various storage media presented herein include one or more devices and/or other machine-readable media for storing information. The term “machine-readable” includes, wireless channels and various other media capable of storing, retaining, and/or communicating command(s) and/or data, but are not limited thereto.

It is understood that the particular order or hierarchy structure of the steps in the processes presented are examples of exemplary approaches. It is understood that the particular order or hierarchy structure in the processes may be rearranged within the scope of the present disclosure based on design priorities. The appended method claims provide elements of various steps in a sample order, but are not meant to be limited to the particular order or hierarchy structure presented.

The description of the embodiments presented is provided to enable one of ordinary skill in the art of the present invention to use or practice the present invention. Various modifications to these embodiments will be apparent to one of ordinary skill in the art, and the general principles defined herein may be applied to other embodiments without departing from the scope of the present invention. Accordingly, the present invention is not to be limited to the embodiments set forth herein, but is to be interpreted in the broadest scope consistent with the principles and novel features set forth herein.

1. A method for adjusting an object's environment, comprising the steps of:
acquiring environment sensing information;
pre-processing the acquired environment sensing information;
converting sound information included in the pre-processed environment sensing information into a spectrogram;
generating sleep state information based on the converted spectrogram; and
controlling an electronic device configured to adjust the object's environment based on the generated sleep state information.

2. An electronic device for adjusting an object's environment, comprising:
a sensor configured to acquire environment sensing information;
means for pre-processing the acquired environment sensing information;
means for converting sound information included in the pre-processed environment sensing information into a spectrogram;
means for generating sleep state information based on the converted spectrogram; and
means for controlling the electronic device configured to adjust the object's environment based on the generated sleep state information.

3. An electronic device for adjusting an object's environment, comprising:
a sensor configured to acquire environment sensing information;
means for pre-processing the acquired environment sensing information;
means for converting sound information included in the pre-processed environment sensing information into a spectrogram;
means for transmitting the converted spectrogram to a server;
wherein the server generates sleep state information based on the transmitted spectrogram,
means for receiving the generated sleep state information; and
means for controlling the electronic device configured to adjust the object's environment based on the received sleep state information.

4. An electronic device for controlling a home appliance configured to adjust an object's environment, comprising:
a sensor configured to acquire environment sensing information;
means for pre-processing the acquired environment sensing information;
means for converting sound information included in the pre-processed environment sensing information into a spectrogram;
means for generating sleep state information based on the converted spectrogram; and
means for controlling the home appliance configured to adjust the object's environment based on the generated sleep state information.

5. An electronic device for controlling a home appliance configured to adjust an object's environment, comprising:
a sensor configured to acquire environment sensing information;
means for pre-processing the acquired environment sensing information;
means for converting sound information included in the pre-processed environment sensing information into a spectrogram;
means for transmitting the converted spectrogram to a server;
wherein the server generates sleep state information based on the transmitted spectrogram,
means for receiving the generated sleep state information; and
means for controlling the home appliance configured to adjust the object's environment based on the received sleep state information.

6. An electronic device for controlling a home appliance configured to adjust an object's environment, comprising:
wherein another electronic device acquires environment sensing information, converts sound information included in the acquired environment sensing information into a spectrogram, and generates sleep state information based on the converted spectrogram,
means for receiving the sleep state information generated in the another electronic device; and
means for controlling the home appliance configured to adjust the object's environment based on the received sleep state information.

7. An electronic device for controlling a home appliance configured to adjust an object's environment, comprising:
wherein another electronic device acquires environment sensing information, converts sound information included in the acquired environment sensing information into a spectrogram, transmits the converted spectrogram to a server, and
wherein the server generates sleep state information based on the transmitted spectrogram,
means for receiving the generated sleep state information from the server; and
means for controlling the home appliance configured to adjust the object's environment based on the received sleep state information.

8. The method of claim 1,
wherein the sound information includes breathing sound.

9. The method of claim 1,
wherein the spectrogram includes a plurality of spectrograms generated by dividing the spectrogram into 30 second units.

10. The method of claim 1,
wherein the sleep state information includes sleep stage information.

11. The electronic device of claim 2,
wherein the sound information includes breathing sound.

12. The electronic device of claim 2,
wherein the spectrogram includes a plurality of spectrograms generated by dividing the spectrogram into 30 second units.

13. The electronic device of claim 2,
wherein the sleep state information includes sleep stage information.

14. The electronic device of claim 4,
wherein the sound information includes breathing sound.

15. The electronic device of claim 4,
wherein the spectrogram includes a plurality of spectrograms generated by dividing the spectrogram into 30 second units.

16. The electronic device of claim 4,
wherein the sleep state information includes sleep stage information.
